(19) United States
(12) Patent Application Publication
(10) Pub. No.: US 2021/0356455 A1
(43) Pub. Date: Nov. 18, 2021

(54) SCREENING AGENTS CAPABLE OF INHIBITING PAIN AND/OR PRURITUS AND METHODS AND COMPOSITIONS FOR TREATING PAIN AND/OR PRURITUS USING SAID AGENTS

(71) Applicant: President and Fellows of Harvard College, Cambridge, MA (US)

(72) Inventors: David D. Ginty, Cambridge, MA (US); Nikhil Sharma, Cambridge, MA (US)

(73) Assignee: President and Fellows of Harvard College, Cambridge, MA (US)

(21) Appl. No.: 17/322,861

(22) Filed: May 17, 2021

Related U.S. Application Data
(60) Provisional application No. 63/025,910, filed on May 15, 2020.

(51) Int. Cl. G01N 33/50; A61K 49/00

(52) U.S. Cl. CPC G01N 33/5058; A61K 49/0008

(57) ABSTRACT

The present disclosure provides a newly conceived of platform for identifying novel pain and/or pruritus-inhibiting agents (e.g., small molecule compounds, peptides, antigen binding proteins (e.g., antibodies or antibody fragments) that are efficacious, safe, and non-addictive alternatives for pain and pruritus management in place of (or in some embodiments, in combination with) “first line” treatments, such as gabapentin, pregabalin, and opioids. The present disclosure also provides for a method of treating pain and/or a pruritus comprising administering a therapeutically effective amount of an agent that activates a G_{ai/o}-coupled G-Protein Coupled Receptor (GPCR) that is selectively expressed in the nociceptor and/or pruriceptor neuron subtypes of the somatosensory neurons. The disclosure also provides a method of inhibiting a nociceptor and/or pruriceptor somatosensory neurons, comprising contacting the nociceptor and/or pruriceptor somatosensory neuron with an agonist of a G-Protein Coupled Receptor (GPCR) expressed on the nociceptor and/or pruriceptor somatosensory neuron.

### RELATED APPLICATIONS

This application claims the benefit under 35 U.S.C. § 119(e)(3) to U.S. provisional patent application No. 63/025,910, filed May 15, 2020, the entire contents of which are incorporated herein by reference.

### GOVERNMENT SUPPORT

This invention was made with government support under grant number NS97344 awarded by the National Institutes of Health. The government has certain rights in the invention.

### BACKGROUND OF THE INVENTION

The perception of pain relies on primary sensory neurons that innervate the skin and other peripheral organs. The current understanding of the mechanisms by which noxious stimuli are detected and conveyed by primary sensory neurons to the central nervous system is remarkably deficient. This has resulted in an innovation gap in developing new therapeutic approaches to pain, leaving few treatment options for prevalent diseases leading to debilitating pain and itch as found in painful diabetic neuropathy (PDN, ˜9,000 cases per 100,000 in the U.S.) or chronic pruritus (7,000 cases per 100,000) (i.e., chronic itch). The current standard of care for these two disorders alone represents a market of nearly $10B, however the treatments for these, as well as a majority of pain disorders, have remained unchanged for decades. “First-line” treatment options include the anticonvulsants gabapentin and pregabalin, which have poor efficacy and serious side effects, while other treatment options involve the alarming use of opioids, contributing to the addiction epidemic. In light of limited treatment options for pain disorders and pruritus, there is a significant unmet need for therapeutics that are efficacious, safe, and non-addictive alternatives for pain and pruritus management. Methods for identifying such therapeutic agents for use as efficacious, safe, and non-addictive alternative treatments for pain and chronic itching, as well as novel alternative therapeutic agents, would significantly advance the art over first-line treatments available at present.

### SUMMARY

In one aspect, the present disclosure provides a newly conceived of platform for identifying novel pain and/or pruritus-inhibiting agents (e.g., small molecule compounds, peptides, antigen binding proteins (e.g., antibodies or antibody fragments) that are efficacious, safe, and non-addictive alternatives for pain and pruritus management in place of (or in some embodiments, in combination with) “first line” treatments, such as gabapentin, pregabalin, and opioids. In another aspect, the present disclosure provides methods for treating pain and/or pruritus with pain and/or pruritus-inhibiting agents (e.g., small molecule compounds, peptides, antigen binding proteins (e.g., antibodies or antibody fragments) that are efficacious, safe, and non-addictive alternatives for pain and pruritus management in place of (or in some embodiments, in combination with) “first line” treatments, such as gabapentin, pregabalin, and opioids.

The present disclosure is based, in part, on the inventors' discovery that certain G-protein coupled receptors (GPCRs), and in particular, those which are coupled to the G_{ai/o}-signaling pathway, are restricted in expression to certain nociceptor and pruriceptor subtypes, but not proprioceptors, mechanoreceptors, or other sensory neurons subtypes that are not involved in conveying pain and/or itch sensations. In particular, the present inventors herein discovered and describe at least six transcriptionally distinct cellular subtypes of nociceptors and at least two transcriptionally distinct cellular subtypes of pruriceptors, and revealed a set of new therapeutic GPCR targets showing restricted expression in said nociceptor and/or pruriceptor subtypes which may be selectively activated to inhibit pain and/or itch processing pathways, without affecting other sensory pathways (e.g., sensing through proprioceptors or mechanoceptors).

The present inventors show in FIG. 27 scRNA-seq analysis of all subtypes of DRG sensory neurons from progenitors to adulthood. The CGRP+ subsets are nociceptors (including CGRP-Theta (CGRP-θ), CGRP-Eta (CGRP-η), CGRP-Zeta (CGRP-ζ), CGRP-Gamma (CGRP-γ), CGRP-Epsilon (CGRP-ε), and CGRP-Alpha (CGRP-α)), whereas the Somatostatin+ neurons are pruriceptors (including Somatostatin (Sst)).

The inventors describe in FIG. 29A a set of the discovered GPCR targets having expression restricted to nociceptor and/or pruriceptor subtypes, such as nociceptor subtypes CGRP-Theta (CGRP-θ), CGRP-Eta (CGRP-η), CGRP-Zeta (CGRP-ζ), CGRP-Gamma (CGRP-γ), CGRP-Epsilon (CGRP-ε), and CGRP-Alpha (CGRP-α)) sensory neurons and pruriceptor subtypes somatostatin+ sensory neurons. In particular, FIG. 29A shows the following identified GPCRs: ADGRA1, ADGRD1, ADGRE5, ADGRF5, ADORA2A, ADORA2B, ADRA2A, ADRA2C, AGTR1A, AGTR1B, AGTRAP, AVPR1A, CALCRL, CELSR2, CESLR3, CHRM1, CRCP, CYSLTR2, DRD1, F2RL1, F2RL2, FZD3, FZD5, FZD8, GALR1, GHSR, GPR35, GPR149, GPR156, GPR173, GPR174, GPR19, GPR4, GRM5, GRM7, HCRTR1, HTR1B, HTR1F, HTR4, HTR5A, LPAR1, LPAR3, MRGPRA1, MRGPRA3, MRGPRA4, MRGPRB4, MRGPRB5, MRGRPD, MRGPRE, MRGPRX1, NPY1R, NYP2R, OGFR, OLFR139, OPN3, OPRK1, OPRM1, OXTR, PROKR2, PTAFR, PTGDR, PTGER1, PTGER2, PTGER3, PTGFR, PTGIR, RAMP3, RHO, S1PR1, S1PR2, S1PR3, SSTR2, VMN1R85, and VMN1R89. This group of GPCRs can be used in certain embodiments to screens for agents that activate GPCRs. In some embodiments, the activation of the GPCR is coupled to the G_{ai/o}-signaling pathway. The agents can be in various embodiments, small molecules, peptides, or antigen binding proteins which specifically bind to and activate GPCR. Concomitantly, the activation of GPCR (which may be coupled to G_{ai/o}-signaling pathway) will inhibit nociceptor and/or pruriceptor subtypes which were identified by the inventors to show restricted expression of said GPCRs.

The inventors further show in FIG. 29B a set of the herein discovered GPCR targets having expression restricted to nociceptor and/or pruriceptor subtypes, such as nociceptor subtypes CGRP-Theta (CGRP-θ), CGRP-Eta (CGRP-η), CGRP-Zeta (CGRP-ζ), CGRP-Gamma (CGRP-γ), CGRP-Epsilon (CGRP-ε), and CGRP-Alpha (CGRP-α)) sensory neurons and pruriceptor subtypes somatostatin+ sensory neurons. In particular, FIG. 29B shows the identification of GPCRs of ADRA2C, GPR35, GPR149, HTR1B, and PTGFR. This subgroup of GPCRs can be used in certain embodiments to screens for agents that activate GPCRs. In some embodiments, the activation of the GPCR is coupled to the G_{ai/o}-signaling pathway. The agents can be in various embodiments, small molecules, peptides, or antigen binding proteins which specifically bind to and activate GPCR. Concomitantly, the activation of GPCR (which may be coupled to G_{ai/o}-signaling pathway) will inhibit nociceptor and/or pruriceptor subtypes which were identified by the inventors to show restricted expression of said GPCRs.

Through their work, the inventors determined that an ideal GPCR to be used as a target for an agent (e.g., small molecule compound, peptide, or antigen binding protein) that is useful for treating pain and/or itch may have one or more of the following properties, and in some embodiments, all of the following properties: (1) the GPCR is highly expressed in nociceptors, pruriceptors, or combinations of both; (2) the GPCR is coupled to the G_{ai/o} signaling pathway; (3) the GPCR exhibits a conserved pattern of expression between rodent and human DRGs; (4) the GPCR is expressed at low levels in other sensory neuron subtypes (e.g., mechanoreceptors, proprioceptors, or other peripheral sensory neurons that convey sensations such as temperature, pressure, and limb movement or position, excluding pain and/or itch sensations), as well as low levels of expression in the peripheral tissues and/or the brain; and (5) activation of the GPCR attenuates pain or itch perception, in particular, where the GPCR is coupled to the G_{ai/o} signaling pathway.

Thus, in various aspects, the present disclosure provides (1) a screening platform for identifying novel pain and/or pruritus-inhibiting agents (e.g., small molecule compounds, peptides, antigen binding proteins (e.g., antibodies or antibody fragments) that is based on the inventors' discovery that certain GPCRs are restricted in expression to one or more subsets of nociceptors and pruriceptors, but not proprioceptors or other sensory neurons subtypes not involved in pain and/or itch detection. In certain embodiments, that GPCRs are coupled to the G_{ai/o} signaling pathway. The present disclosure further provides (2) identified pain and/or pruritus-inhibiting agents (e.g., small molecule compounds, peptides, antigen binding proteins (e.g., antibodies or antibody) which (i) activate certain GPCRs (e.g., those of FIG. 29A and FIG. 29B) which show restricted expression in one or more subsets of nociceptors and pruriceptors (e.g., sensory neurons identified as CGRP-Theta (CGRP-θ), CGRP-Eta (CGRP-η), CGRP-Zeta (CGRP-ζ), CGRP-Gamma (CGRP-γ), CGRP-Epsilon (CGRP-ε), and CGRP-Alpha (CGRP-α)) nociceptor sensory neurons and somatostatin+ sensory pruriceptor neurons), but not proprioceptors or other sensory neurons subtypes not involved in pain and/or itch perception. In some embodiments, the agents can be known GPCR agonists which are tested and confirmed to activate one or more of the nociceptor-specific or pruripotent-specific GPCRs identified by the inventors (e.g., those of FIG. 29A and FIG. 29B). In other embodiments, the agents can be previously known agents, but which were not previously known to bind to and activate one or more of the nociceptor-specific or pruripotent-specific GPCRs identified by the inventors (e.g., those of FIG. 29A and FIG. 29B). In still other embodiments, the agents can be novel agents, but which did not previously exist in the art and which were or can be shown to bind to and activate one or more of the nociceptor-specific or pruripotent-specific GPCRs identified by the inventors (e.g., those of FIG. 29A and FIG. 29B).

Accordingly, in various embodiments, the disclosure provides methods for screening for agents from a plurality of candidate agents (a library of small molecules, peptides, or antigen binding proteins (e.g., antibodies), wherein said agents are pain and/or pruritus-inhibiting agents (e.g., small molecule compounds, peptides, antigen binding proteins (e.g., antibodies or antibody fragments) that bind to and activate one or more GPCRs which are restricted in expression to one or more subsets of nociceptors and pruriceptors but which are not expressed in proprioceptors or other sensory neuron subtypes not involved in pain and/or itch detection. In certain embodiments, that GPCRs are coupled to the G_{ai/o} signaling pathway. In various embodiments, the disclosure also provides methods of testing and confirming whether a given GPCR is coupled to the G_{ai/o} signaling pathway.

In still other embodiments, the present disclosure provides libraries of candidate agents, e.g., small molecule libraries, peptide libraries, antibody libraries, etc. which may be screened using the methods disclosed herein to assay for binding to and activating one or more GPCRs (e.g., the GPCRs of FIG. 29A or 29B) which are restricted in expression to one or more subsets of nociceptors and pruriceptors but which are not expressed in proprioceptors or other sensory neuron subtypes not involved in pain and/or itch detection.

In yet other embodiments, the present disclosure provides nucleic acid molecules encoding the nociceptor and/or pruriceptor-specific GPCRs (including those which are coupled to the G_{ai/o} signaling pathway), and to cloning and/or expression vectors comprising said nucleic acid molecules encoding the nociceptor and/or pruriceptor-specific GPCRs. Further, the disclosure provides for cells comprising said cloning and/or expression vector comprising said nucleic acid molecules encoding the nociceptor and/or pruriceptor-specific GPCRs.

In various embodiments, the disclosure also provides for various reagents, biochemical assays, etc. capable of detecting when a particular candidate agent binds to and/or activates a nociceptor and/or pruriceptor-specific GPCR (e.g., one or more of those GPCRs of FIG. 29A or 29B).

In still further embodiments, the disclosure also provides for various reagents, biochemical assays, etc. capable of detecting when a particular candidate agent binds to and/or activates a nociceptor and/or pruriceptor-specific GPCR (e.g., one or more of those GPCRs of FIG. 29A or 29B). Such reagents and/or biochemical assays may be used in connection with in vitro and/or in vivo assays.

In yet further embodiments, the disclosure also provides for various animal models capable of detecting when a particular candidate agent binds to and/or activates a nociceptor and/or pruriceptor-specific GPCR (e.g., one or more of those GPCRs of FIG. 29A or 29B) and further, whether said candidate agents result in the inhibition of pain and/or itch perception in the animal model.

The following embodiments are within the scope of the present disclosure. Furthermore, the disclosure encompasses all variations, combinations, and permutations of these embodiments in which one or more limitations, elements, clauses, and descriptive terms from one or more of the listed embodiments is introduced into another listed embodiment in this section. For example, any listed embodiment that is dependent on another embodiment can be modified to include one or more limitations found in any other listed embodiment in this section that is dependent on the same base embodiment. Where elements are presented as lists, e.g., in Markush group format, each subgroup of the elements is also disclosed, and any element(s) can be removed from the group. It should it be understood that, in general, where the disclosure, or aspects of the disclosure, is/are referred to as comprising particular elements and/or features, certain embodiments of the invention or aspects of the invention consist, or consist essentially of, such elements and/or features. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

Embodiment 1. A method of treating pain and/or a pruritus comprising administering a therapeutically effective amount of an agent that activates a G_{ai/o}-coupled G-Protein Coupled Receptor (GPCR) that is selectively expressed in the nociceptor and/or pruriceptor neuron subtypes of the somatosensory neurons.

Embodiment 2. The method of embodiment 1, wherein as a result of the activation of the G_{ai/o}-coupled G-Protein Coupled Receptor (GPCR), the pain and/or itch signaling by the nociceptor and/or pruriceptor neuron subtypes is reduced and/or blocked.

Embodiment 3. The method of embodiment 1, wherein the G_{ai/o}-coupled G-Protein Coupled Receptor (GPCR) is selectively expressed in the nociceptor and/or pruriceptor neuron subtypes of the somatosensory neurons, but not expressed or expressed at low levels in other somatosensory neuron subtypes, peripheral tissues, and/or brain.

Embodiment 4. The method of embodiment 1, wherein the G_{ai/o}-coupled G-Protein Coupled Receptor (GPCR) is selectively expressed in the nociceptor and/or pruriceptor neuron subtypes of the somatosensory neurons, but not detectable in other somatosensory neuron subtypes, peripheral tissues, and/or brain.

Embodiment 5. The method of embodiment 1, wherein the G_{ai/o}-coupled G-Protein Coupled Receptor (GPCR) is selected from the group consisting of: ADGRA1, ADGRD1, ADGRE5, ADGRF5, ADORA2A, ADORA2B, ADRA2A, ADRA2C, AGTR1A, AGTR1B, AGTRAP, AVPR1A, CALCRL, CELSR2, CESLR3, CHRM1, CRCP, CYSLTR2, DRD1, F2RL1, F2RL2, FZD3, FZD5, FZD8, GALR1, GHSR, GPR35, GPR149, GPR156, GPR173, GPR174, GPR19, GPR4, GRM5, GRM7, HCRTR1, HTR1B, HTR1F, HTR4, HTR5A, LPAR1, LPAR3, MRGPRA1, MRGPRA3, MRGPRA4, MRGPRB4, MRGPRB5, MRGRPD, MRGPRE, MRGPRX1, NPY1R, NYP2R, OGFR, OLFR139, OPN3, OPRK1, OPRM1, OXTR, PROKR2, PTAFR, PTGDR, PTGER1, PTGER2, PTGER3, PTGFR, PTGIR, RAMP3, RHO, S1PR1, S1PR2, S1PR3, SSTR2, VMN1R85, and VMN1R89.

Embodiment 6. A method of embodiment 1, wherein the agent is identified by performing a high throughput compound screen for molecules that activate the G_{ai/o}-coupled G-Protein Coupled Receptor (GPCR).

Embodiment 7. The method of embodiment 1, wherein the agent is a known ligand of the G_{ai/o}-coupled G-Protein Coupled Receptor (GPCR).

Embodiment 8. The method of embodiment 1, wherein the activation of the G_{ai/o}-coupled G-Protein Coupled Receptor (GPCR) causes downstream activation of G-protein coupled inwardly rectifying potassium channels (GIRKs).

Embodiment 9. The method of embodiment 8, wherein the activation of the GIRKS causes silencing of neuronal activity of the nociceptor and/or pruriceptor neuron subtypes.

Embodiment 10. A method of treating pain and/or a pruritus comprising administering a therapeutically effective amount of an agent that activates a G_{ai/o}-coupled G-Protein Coupled Receptor (GPCR) that is selectively expressed in the nociceptor and/or pruriceptor neuron subtypes of the somatosensory neurons.

Embodiment 11. The method of embodiment 10, wherein as a result of the activation of the G_{ai/o}-coupled G-Protein Coupled Receptor (GPCR), the pain and/or itch signaling by the nociceptor and/or pruriceptor neuron subtypes is reduced and/or blocked.

Embodiment 12. The method of embodiment 10, wherein the G_{ai/o}-coupled G-Protein Coupled Receptor (GPCR) is selectively expressed in the nociceptor and/or pruriceptor neuron subtypes of the somatosensory neurons, but not expressed or expressed at low levels in other somatosensory neuron subtypes, peripheral tissues, and/or brain.

Embodiment 13. The method of embodiment 10, wherein the G_{ai/o}-coupled G-Protein Coupled Receptor (GPCR) is selectively expressed in the nociceptor and/or pruriceptor neuron subtypes of the somatosensory neurons, but not detectable in other somatosensory neuron subtypes, peripheral tissues, and/or brain.

Embodiment 14. The method of embodiment 10, wherein the G_{ai/o}-coupled G-Protein Coupled Receptor (GPCR) is selected from the group consisting of: ADGRA1, ADGRD1, ADGRE5, ADGRF5, ADORA2A, ADORA2B, ADRA2A, ADRA2C, AGTR1A, AGTR1B, AGTRAP, AVPR1A, CALCRL, CELSR2, CESLR3, CHRM1, CRCP, CYSLTR2, DRD1, F2RL1, F2RL2, FZD3, FZD5, FZD8, GALR1, GHSR, GPR35, GPR149, GPR156, GPR173, GPR174, GPR19, GPR4, GRM5, GRM7, HCRTR1, HTR1B, HTR1F, HTR4, HTR5A, LPAR1, LPAR3, MRGPRA1, MRGPRA3, MRGPRA4, MRGPRB4, MRGPRB5, MRGRPD, MRGPRE, MRGPRX1, NPY1R, NYP2R, OGFR, OLFR139, OPN3, OPRK1, OPRM1, OXTR, PROKR2, PTAFR, PTGDR, PTGER1, PTGER2, PTGER3, PTGFR, PTGIR, RAMP3, RHO, S1PR1, S1PR2, S1PR3, SSTR2, VMN1R85, and VMN1R89.

Embodiment 15. A method of embodiment 10, wherein the agent is identified by performing a high throughput compound screen for molecules that activate the G_{ai/o}-coupled G-Protein Coupled Receptor (GPCR).

Embodiment 16. The method of embodiment 10, wherein the agent is a known ligand of the G_{ai/o}-coupled G-Protein Coupled Receptor (GPCR).

Embodiment 17. The method of embodiment 10, wherein the activation of the G_{ai/o}-coupled G-Protein Coupled Receptor (GPCR) causes downstream activation of G-protein coupled inwardly rectifying potassium channels (GIRKs).

Embodiment 18. The method of embodiment 17, wherein the activation of the GIRKS causes silencing of neuronal activity of the nociceptor and/or pruriceptor neuron subtypes.

Embodiment 19. A method of inhibiting a nociceptor and/or pruriceptor somatosensory neuron, comprising contacting the nociceptor and/or pruriceptor somatosensory neuron with an agonist of a G-Protein Coupled Receptor (GPCR) expressed on the nociceptor and/or pruriceptor somatosensory neuron.

Embodiment 20. The method of embodiment 19, wherein the inhibiting results in the reduction or blocking of pain and/or itch signaling by the nociceptor and/or somatosensory pruriceptor neurons.

Embodiment 21. The method of embodiment 19, wherein the G-Protein Coupled Receptor (GPCR) is a G_{ai/o}-coupled GPCR.

Embodiment 22. The method of claim embodiment 21, wherein the G_{ai/o}-coupled GPCR is selectively expressed in the nociceptor and/or pruriceptor somatosensory neuron, but not expressed or expressed at low levels in other somatosensory neurons, peripheral tissues, and/or brain.

Embodiment 23. The method of embodiment 21, wherein the G_{ai/o}-coupled GPCR is selectively expressed in the nociceptor and/or pruriceptor somatosensory neuron, but not detectable in other somatosensory neurons, peripheral tissues, and/or brain.

Embodiment 24. The method of embodiment 19 wherein the agonist activates the G-Protein Coupled Receptor (GPCR), thereby inhibiting a nociceptor and/or pruriceptor somatosensory neuron.

Embodiment 25. The method of embodiment 19, wherein the G_{ai/o}-coupled G-Protein Coupled Receptor (GPCR) is selected from the group consisting of: ADGRA1, ADGRD1, ADGRE5, ADGRF5, ADORA2A, ADORA2B, ADRA2A, ADRA2C, AGTR1A, AGTR1B, AGTRAP, AVPR1A, CALCRL, CELSR2, CESLR3, CHRM1, CRCP, CYSLTR2, DRD1, F2RL1, F2RL2, FZD3, FZD5, FZD8, GALR1, GHSR, GPR35, GPR149, GPR156, GPR173, GPR174, GPR19, GPR4, GRM5, GRM7, HCRTR1, HTR1B, HTR1F, HTR4, HTR5A, LPAR1, LPAR3, MRGPRA1, MRGPRA3, MRGPRA4, MRGPRB4, MRGPRB5, MRGRPD, MRGPRE, MRGPRX1, NPY1R, NYP2R, OGFR, OLFR139, OPN3, OPRK1, OPRM1, OXTR, PROKR2, PTAFR, PTGDR, PTGER1, PTGER2, PTGER3, PTGFR, PTGIR, RAMP3, RHO, S1PR1, S1PR2, S1PR3, SSTR2, VMN1R85, and VMN1R89.

Embodiment 26. A method of embodiment 19, wherein the agonist is identified by performing a high throughput compound screen for molecules that activate the G-Protein Coupled Receptor (GPCR).

Embodiment 27. The method of embodiment 19, wherein the agonist is a known ligand of the G-Protein Coupled Receptor (GPCR).

Embodiment 28. The method of embodiment 24, wherein the activation of the G-Protein Coupled Receptor (GPCR) causes downstream activation of G-protein coupled inwardly rectifying potassium channels (GIRKs).

Embodiment 29. The method of embodiment 28, wherein the activation of the GIRKS causes silencing of the nociceptor and/or pruriceptor somatosensory neuron.

Embodiment 30. A method of screening to identify an agent that selectively inhibits primary nociceptors to attenuate pain perception, said method comprising contacting a G-Protein Coupled Receptor (GPCR) that is selectively expressed in said nociceptors relative to other subtypes of somatosensory neurons with a candidate agent and detecting whether said candidate agent activates the G-Protein Coupled Receptor.

Embodiment 31. The method of embodiment 30, wherein the G-Protein Coupled Receptor is highly expressed in said nociceptors but expressed at low levels in other subtypes of somatosensory neurons.

Embodiment 32. The method of embodiment 31, wherein the G-Protein Coupled Receptor is expressed at low levels in peripheral tissues and/or brain.

Embodiment 33. The method of embodiment 30, wherein the G-Protein Coupled Receptor is coupled to the G_{ai/o}-signaling pathway.

Embodiment 34. The method of embodiment 30, wherein the G-Protein Coupled Receptor exhibits a conserved pattern of expression between rodent and human dorsal root ganglia (DRG).

Embodiment 35. The method of embodiment 30, wherein the G-Protein Coupled Receptor is selected from the group consisting of ADGRA1, ADGRD1, ADGRE5, ADGRF5, ADORA2A, ADORA2B, ADRA2A, ADRA2C, AGTR1A, AGTR1B, AGTRAP, AVPR1A, CALCRL, CELSR2, CESLR3, CHRM1, CRCP, CYSLTR2, DRD1, F2RL1, F2RL2, FZD3, FZD5, FZD8, GALR1, GHSR, GPR35, GPR149, GPR156, GPR173, GPR174, GPR19, GPR4, GRM5, GRM7, HCRTR1, HTR1B, HTR1F, HTR4, HTR5A, LPAR1, LPAR3, MRGPRA1, MRGPRA3, MRGPRA4, MRGPRB4, MRGPRB5, MRGRPD, MRGPRE, MRGPRX1, NPY1R, NYP2R, OGFR, OLFR139, OPN3, OPRK1, OPRM1, OXTR, PROKR2, PTAFR, PTGDR, PTGER1, PTGER2, PTGER3, PTGFR, PTGIR, RAMP3, RHO, S1PR1, S1PR2, S1PR3, SSTR2, VMN1R85, and VMN1R89.

Embodiment 36. The method of embodiment 30, wherein the G-Protein Coupled Receptor is ADRA2C.

Embodiment 37. The method of embodiment 30, wherein the G-Protein Coupled Receptor is GPR35.

Embodiment 38. The method of embodiment 30, wherein the G-Protein Coupled Receptor is GPR149.

Embodiment 39. The method of embodiment 30, wherein the G-Protein Coupled Receptor is HTR1B.

Embodiment 40. The method of embodiment 30, wherein the G-Protein Coupled Receptor is PTGFR.

Embodiment 41. The method of embodiment 30, wherein the candidate agent is a small molecule compound, peptide, antigen binding protein, or nucleic acid molecule.

Embodiment 42. The method of embodiment 30, wherein the method of screening comprises an in vitro based assay.

Embodiment 43. The method of embodiment 30, wherein the in vitro based assay comprises contacting the candidate agent with a cell line expressing said G-Protein Coupled Receptor and detecting activation of the G-Protein Coupled Receptor.

Embodiment 44. The method of embodiment 30, wherein the method of screening comprises an in vivo based assay.

Embodiment 45. The method of embodiment 30, further comprising confirming the selectivity of the candidate agent by detecting no or minimal activation of one or more known control G-Protein Coupled Receptors by the candidate agent.

Embodiment 46. The method of embodiment 30, wherein the in vivo based assay comprises testing the efficacy of said candidate agent in attenuating pain perception in an animal model.

Embodiment 47. The method of embodiment 30, wherein the agent is a small molecule compound.

Embodiment 48. The method of embodiment 30, wherein the agent is a peptide.

Embodiment 49. The method of embodiment 30, wherein the agent is an antigen binding protein.

Embodiment 50. The method of embodiment 33, wherein the agent inhibits the G_{ai/o}-signaling pathway.

Embodiment 51. A method of screening to identify an agent that selectively inhibits primary pruriceptors to attenuate itch perception, said method comprising contacting a G-Protein Coupled Receptor that is selectively expressed in said pruriceptors relative to other subtypes of somatosensory neurons with a candidate agent and detecting whether said candidate agent activates the G-Protein Coupled Receptor.

Embodiment 52. The method of embodiment 51, wherein the G-Protein Coupled Receptor is highly expressed in said pruriceptors but expressed at low levels in other subtypes of somatosensory neurons.

Embodiment 53. The method of embodiment 51, wherein the G-Protein Coupled Receptor is expressed at low levels in peripheral tissues and/or brain.

Embodiment 54. The method of embodiment 51, wherein the G-Protein Coupled Receptor is coupled to the G_{ai/o}-signaling pathway.

Embodiment 55. The method of embodiment 51, wherein the G-Protein Coupled Receptor exhibits a conserved pattern of expression between rodent and human dorsal root ganglia (DRG).

Embodiment 56. The method of embodiment 51, wherein the G-Protein Coupled Receptor is selected from the group consisting of ADGRA1, ADGRD1, ADGRE5, ADGRF5, ADORA2A, ADORA2B, ADRA2A, ADRA2C, AGTR1A, AGTR1B, AGTRAP, AVPR1A, CALCRL, CELSR2, CESLR3, CHRM1, CRCP, CYSLTR2, DRD1, F2RL1, F2RL2, FZD3, FZD5, FZD8, GALR1, GHSR, GPR35, GPR149, GPR156, GPR173, GPR174, GPR19, GPR4, GRM5, GRM7, HCRTR1, HTR1B, HTR1F, HTR4, HTR5A, LPAR1, LPAR3, MRGPRA1, MRGPRA3, MRGPRA4, MRGPRB4, MRGPRB5, MRGRPD, MRGPRE, MRGPRX1, NPY1R, NYP2R, OGFR, OLFR139, OPN3, OPRK1, OPRM1, OXTR, PROKR2, PTAFR, PTGDR, PTGER1, PTGER2, PTGER3, PTGFR, PTGIR, RAMP3, RHO, S1PR1, S1PR2, S1PR3, SSTR2, VMN1R85, and VMN1R89.

Embodiment 57. The method of embodiment 51, wherein the G-Protein Coupled Receptor is ADRA2C.

Embodiment 58. The method of embodiment 51, wherein the G-Protein Coupled Receptor is GPR35.

Embodiment 59. The method of embodiment 51, wherein the G-Protein Coupled Receptor is GPR149.

Embodiment 60. The method of embodiment 51, wherein the G-Protein Coupled Receptor is HTR1B.

Embodiment 61. The method of embodiment 51, wherein the G-Protein Coupled Receptor is PTGFR.

Embodiment 62. The method of embodiment 51, wherein the candidate agent is a small molecule compound.

Embodiment 63. The method of embodiment 51, wherein the method of screening comprises an in vitro based assay.

Embodiment 64. The method of embodiment 63, wherein the in vitro based assay comprises contacting the candidate agent with a cell line expressing said G-Protein Coupled Receptor and detecting activation of the G-Protein Coupled Receptor.

Embodiment 65. The method of embodiment 51, wherein the method of screening comprises an in vivo based assay.

Embodiment 66. The method of embodiment 51, further comprising confirming the selectivity of the candidate agent by detecting no or minimal activation of one or more known control G-Protein Coupled Receptors which are expressed in cells other than pruriceptors.

Embodiment 67. The method of embodiment 65, wherein the in vivo based assay comprises testing the efficacy of said candidate agent in attenuating itch perception in an animal model.

Embodiment 68. The method of embodiment 51, wherein the candidate agent is a small molecule compound.

Embodiment 69. The method of embodiment 51, wherein the method of screening comprises an in vitro based assay.

Embodiment 70. The method of embodiment 69, wherein the in vitro based assay comprises contacting the candidate agent with a cell line expressing said G-Protein Coupled Receptor and detecting activation of the G-Protein Coupled Receptor.

Embodiment 71. The method of embodiment 51, wherein the method of screening comprises an in vivo based assay.

Embodiment 72. The method of embodiment 51, further comprising confirming the selectivity of the candidate agent by detecting no or minimal activation of one or more known control G-Protein Coupled Receptors by the candidate agent.

Embodiment 73. The method of embodiment 71, wherein the in vivo based assay comprises testing the efficacy of said candidate agent in attenuating pain perception in an animal model.

Embodiment 74. The method of embodiment 51, wherein the agent is a nucleic acid molecule.

Embodiment 75. The method of embodiment 51, wherein the agent is a peptide.

Embodiment 76. The method of embodiment 51, wherein the agent is an antigen binding protein.

Embodiment 77. The method of embodiment 51, wherein the agent inhibits the G_{ai/o}-signaling pathway.

The details of certain embodiments of the invention are set forth in the Detailed Description of Certain Embodiments, as described below. Other features, objects, and advantages of the invention will be apparent from the Definitions, Examples, Figures, and Claims.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIGS. 1A-1D show the scRNA-seq of developing and mature DRG sensory neurons. FIG. 1A shows t-SNE visualizations of DRG scRNA-seq data. FIG. 1B shows a UMAP visualization of DRG scRNA-seq data from E11.5 with developmental trajectory and gene expression information overlaid. TPT: tags per ten thousand. FIG. 1C shows a quantification of tdTomato+ neurons and representative image. Mean+/−s.e.m. is indicated. FIG. 1D shows a heatmap and quantification of genes enriched in each somatosensory neuron subtype as well as their expression levels in unspecialized sensory neurons. USN: unspecialized sensory neuron. Boxes represent IQR, whiskers represent minimum and maximum values, and notches represent the 95% confidence interval of the median. TPT: tags per ten thousand. * denotes two-sided Wilcoxon rank-sum test with Bonferroni corrected p<0.0001.

FIGS. 2A-2C show the transcriptional development of DRG neuron subtypes. FIG. 2A shows a force-directed layout of DRG sensory neurons overlaid with time point (right image) or cell type information (left image). FIG. 2B shows the force-directed layout of DRG development overlaid with expression of indicated genes. FIG. 2C is a heatmap of subtype-restricted TFs in each somatosensory neuron subtype of adult ganglia. TPT: tags per ten thousand.

FIGS. 3A-3F show a refinement of TF expression in developing somatosensory neurons. FIG. 3A shows a developmental trajectory of sensory neurons (left) and tSNE visualization with TF expression overlaid. TPT: tags per ten thousand. FIG. 3B is a schematic description of the Avil^{CreERT2}; Rosa26^{LSL-tdTomato} labeling strategy. FIG. 3C shows a tSNE visualization of Avil^{CreERT2}; Rosa26^{LSL-tdTomato} scRNA-seq with cell type identity or tdTomato expression overlaid. TPT: tags per ten thousand. FIG. 3D is a schematic representing strategy for labeling neurons with Pou4f2^{Cre/WT} mice. FIG. 3E shows the smRNA-FISH and quantification for the indicated transcripts. Mean+/−s.e.m is indicated. * represents two-tailed t-test. p<0.01.

FIGS. 4A-4D show that Pou4f2 and Pou4f3 regulate select somatosensory neuron subtype identities. FIG. 4A shows t-SNE visualizations of scRNA-seq data for neurons generated from Pou4f3^{WT/WT} and Pou4f3^{KO/KO} littermates. FIG. 4B shows t-SNE visualizations of scRNA-seq data for neurons generated from Pou4f2^{WT/WT} and Pou4f2^{KO/KO} littermates. FIG. 4C shows the fold-change distribution of cell-type specific genes when comparing Pou4f3^{KO/KO} and Pou4f3^{WT/WT} littermates control samples. FIG. 4D shows the fold-change distribution of cell-type specific genes when comparing Pou4f2^{KO/KO} and Pou4f2^{WT/WT} littermates. In FIGS. 4C-4D, the boxes represent IQR, whiskers represent minimum and maximum values, and notches represent the 95% confidence interval of the median and * denotes two sided Wilcoxon rank-sum test with Bonferroni corrected p<0.01. TPT: tags per ten thousand.

FIGS. 5A-5C show that the extrinsic cue NGF is required for subtype specific gene expression and TF expression patterns. FIG. 5A shows t-SNE visualizations of scRNA-seq data for neurons generated from P0 Bax^{-/-} and NGF^{-/-}; Bax^{-/-} littermates. FIG. 5B shows cell-type specific gene expression in proprioceptor and A-fiber mechanoreceptor sensory neuron subtypes in Bax^{-/-} and NGF^{-/-}; Bax^{-/-} littermates. FIG. 5C shows cell-type specific gene expression in all other sensory neuron subtypes in Bax^{-/-} and NGF^{-/-}; Bax^{-/-} littermates. Boxes represent IQR, whiskers represent minimum and maximum values, and notches represent the 95% confidence interval of the median. In FIGS. 5B-5C, all clusters are different from controls by two sided Wilcoxon rank-sum test with Bonferroni corrected p<0.01. TPT: tags per ten thousand.

FIGS. 6A-6G show the quality control metrics for DRG sensory neuron scRNA-seq data and canonical correlation analysis. FIGS. 6A-6E show a distribution of the number of genes discovered in each cell (individual points) in each population of sensory neuron (underlying violin plot) in Adult (FIG. 6A), Postnatal day 5 (FIG. 6B), Postnatal day 0 (FIG. 6C), Embryonic day 15.5 (FIG. 6D), and Embryonic day 12.5 (FIG. 6E) mice. Individual cells with <1000 genes discovered (considered to be low quality) or >10000 genes discovered (considered to be likely doublets) were eliminated from subsequent analysis. Individual cells with <2000 UMIs (considered to be low quality) were excluded from subsequent analysis. UMI: unique molecular identifier. FIGS. 6F-6G show an integration of Adult/P5 (1st plot, FIG. 6F), P5/P0 (2nd plot, FIG. 6G), P0/E15.5 (3rd plot, FIG. 6G), and E15.5/E12.5 (4th plot, FIG. 6G) mice, using canonical correlation analysis to find common sources of variation between time points. Single cells are labeled as individual points, with color representing identified cell types and gray representing cells in the preceding time point.

FIGS. 7A-7D show that somatosensory neuron subtype composition varies across axial levels. FIG. 7A (Left) is a schematic showing which axial levels were quantified. FIG. 7A (Right) shows the quantification of single molecule RNA-FISH to determine the percentage of C6/7, T7/8, and L4/5 DRG neurons that correspond to each transcriptionally defined somatosensory neuron subtype. Black dotted lines are used to highlight the subtypes present at different percentages at different axial levels. FIGS. 7B-7D show example images of single molecule RNA-FISH for transcriptionally distinct somatosensory neuron subtypes in C6/7 (FIG. 7B), T7/8 (FIG. 7C) and L4/5 (FIG. 7D) DRGs.

FIGS. 8A-8D show that dorsal root ganglia and trigeminal ganglia are constituted by similar somatosensory neuron subtypes. FIG. 8A shows a tSNE visualization of trigeminal ganglia scRNA-seq data obtained from Adult (P28-42) mice. Colors denote principle cell types and dotted circles were added to aid in visualization of principal cell types. LTMR/proprioceptor specific gene expression overlaid onto t-SNE visualization of mature DRG sensory neurons. FIG. 8B shows the distribution of the number of genes discovered in each population of sensory neuron in adult trigeminal ganglia displayed as violin plot. UMI: unique molecular identifier. FIG. 8C is a heatmap depicting expression of the genes enriched in somatosensory neuron subtypes resident in the dorsal root ganglia as well as their expression levels in cognate trigeminal ganglia subtype counterparts.

FIG. 8D is a heatmap depicting expression of the genes enriched in somatosensory neuron subtypes resident in the trigeminal, as well as their expression levels in cognate dorsal root ganglia subtype counterparts. For FIGS. 8C-8D, boxes represent IQR, whiskers represent minimum and maximum values, and notches represent the 95% confidence interval of the median. TPT: tags per ten thousand. * denotes two sided Wilcoxon rank-sum test with Bonferroni corrected p<0.01. TPT: tags per ten thousand.

FIGS. 9A-9E show that neural crest progenitors, sensory neuron progenitors, and unspecialized sensory neurons express highly distinct gene programs. FIG. 9A is a heatmap depicting cell cycle (S/G2/M) associated genes for the principal subtypes identified at E11.5. FIG. 9B is a heatmap depicting expression levels of unspecialized sensory neurons enriched genes in both mature somatosensory neuron subtypes and unspecialized sensory neurons. FIG. 9C (left) is a heatmap depicting expression of the genes enriched in unspecialized sensory neurons (USN) as well as their expression levels in neural crest progenitors (NCP) and sensory neuron progenitors (SNP). FIG. 9C (right) shows violin and box plots depicting example genes enriched in USNs. FIG. 9D (left) is a heatmap depicting expression of the genes enriched in neural crest progenitors (NCP) as well as their expression levels in sensory neuron progenitors (SNP) and unspecialized sensory neurons (USN). FIG. 9D (right) shows violin and box plots depicting example genes enriched in NCPs. FIG. 9E (left) is a heatmap depicting expression of the genes enriched in sensory neuron progenitors (SNP) as well as their expression levels in neural crest progenitors (NCP) and sensory neuron progenitors (SNP). FIG. 9E (right) shows violin and box plots depicting example genes enriched in SNPs. For FIGS. 9A-9E, boxes represent IQR, whiskers represent minimum and maximum values, and notches represent the 95% confidence interval of the median. TPT: tags per ten thousand. * denotes two sided Wilcoxon rank-sum test with Bonferroni corrected p<0.01.

FIGS. 10A-10C show force directed layouts of putative subtype-restricted transcription factors. FIG. 10A is a force directed layout representation of DRG displaying with expression patterns displayed for the remaining putative subtype-restricted transcription factors. FIG. 9B shows tSNE visualization of Runx1, Runx3, Pou4f2 and Pou4f3 expression in the adult DRG. TPT: tags per ten thousand. FIG. 10C (left) shows single molecule RNA FISH for Runx1 and Runx3 in E11.5, P0 or adult DRGs. For E11.5, the spinal cord and DRG are labeled as references. FIG. 10C (right) shows single molecule RNA FISH for Pou4f2 and Pou4f3 in E11.5, P0 or adult DRGs. For E11.5, the spinal cord and DRG are labeled as references. The graphs on the bottom show a quantification of the RNA-FISH. * represents two-sided t-test p<0.01.

FIGS. 11A-11B show the expression of somatosensory neuron subtype specific genes during development. FIG. 11A shows box plots representing subtype specific genes at E12.5, E15.5, P0, P5 and Adult (P28-42) mice for each identified somatosensory neuron subtype. Boxes represent IQR, whiskers represent minimum and maximum values, and notches represent the 95% confidence interval of the median. TPT: tags per ten thousand. * denotes two sided Wilcoxon rank-sum test with Bonferroni corrected p<0.01. FIG. 11B shows normalized line plots displaying what percentage of adult-levels of subtype specific gene expression are detected at E12.5, E15.5, P0, and P5. The black line represents the median of each time point with Adult being defined as ‘100%’. Upper and lower bands represent a 95% confidence interval (defined as ±1.87*IQR/√n, where n=sample size, IQR: Interquartile range)

FIGS. 12A-12H show DRG counts and TF analysis in Pou4f2 and Pou4f3 mutants. FIG. 12A shows representative images of Avil smRNA-FISH from T7/8 DRGs in Pou4f2^{KO(Cre)/WT} (left) or Pou4f2^{KO(Cre)/KO(Cre)} (right) littermate control DRGs. Quantification of estimated cell count per DRG presented to the right of the images. FIG. 12B shows representative images of Avil smRNA-FISH from T7/8 DRGs in Pou4f3^{WT/WT} (left) or Pou4f3^{KO/KO} (right) littermate control DRGs. Quantification of estimated cell count per DRG presented to the right of the images. FIGS. 12C-12E show box plots displaying the expression levels of subtype-restricted TFs in each somatosensory neuron subtype in Pou4f2^{WT/WT} (left column) or Pou4f2^{KO(Cre)/KO(Cre)} (right column) littermates.

FIGS. 12F-12H shows box plots displaying the expression levels of subtype-restricted TFs in each somatosensory neuron subtype in Pou4f3^{WT/WT} (left column) or Pou4f3^{KO/KO} (right column) littermates. For FIGS. 12C-12H, boxes represent IQR, whiskers represent minimum and maximum values, and notches represent the 95% confidence interval of the median. TPT: tags per ten thousand.

FIGS. 13A-13K show generation and validation of Bmpr1b^{T2a-Cre} and Avpr1a^{T2a-Cre} mouse lines. FIG. 13A shows a targeting strategy for inserting a T2a-Cre-TGA^{STOP codon}; Frt-PGK: Neo^{R} pA-Frt cassette immediately upstream of the stop codon in the Bmpr1b gene. FIG. 13B shows a single molecule RNA-FISH for both Bmpr1b and GFP in Bmpr1b^{T2a-Cre}; AAV-CAG:FLEX-GFP^{P14 LV}. mice to confirm the specificity and utility of the Bmpr1b^{T2a-Cre} allele. FIG. 13C shows a targeting strategy for inserting a T2a-Cre-TGA^{STOP codon}; Frt-PGK: Neo^{R}-pA-Frt cassette immediately upstream of the stop codon in the Avpr1a gene. FIG. 13D shows a single molecule RNA-FISH for both Avpr1a and tdTomato in Avpr1a^{T2a-Cre(ΔNeo)}; Rosa26^{LSL-tdTomato/WT} mice to confirm the specificity and utility of the Avpr1a^{T2a-Cre} allele. FIG. 13E shows (top left) tSNE representation of transcriptionally mature DRG overlaying the expression pattern of Avpr1a, and the remaining images show immunostaining images of tdTomato and CGRP in skin sections obtained from Avpr1a^{T2a-Cre}; Rosa26^{LSL-tdTomato} animals. TPT: tags per ten thousand. FIG. 13F shows (top left) tSNE representation of transcriptionally mature DRG overlaying the expression pattern of Bmpr1b, and the remaining images show immunostaining images of tdTomato and CGRP in skin sections obtained from Bmpr1b^{T2a-Cre}; AAV-CAG:FLEX-GFP^{P14 LV}. animals. TPT: tags per ten thousand. FIG. 13G shows representative immunostaining images of GFP in skin sections obtained from Pou4f2^{KO(Cre)}; AAV-CAG:FLEX-GFP^{P14 LV} animals. FIG. 13H shows a quantification of ending morphology for CGRP-α (Avpr1a^{T2a-Cr}); Rosa26^{LSL-tdTomato}) and CGRP-η (Bmpr1b^{T2a-Cre}; AAV-CAG:FLEX-GFP^{P14 LV}.) somatosensory neuron subtypes. FIG. 13I is a schematic representation of the skin with the distinct morphological ending types of CGRP-α and CGRP-η neurons displayed. FIG. 13J shows representative images of CGRP immunostaining in skin samples of 2-3 week old Pou4f3^{WT/WT} (left) or Pou4f3^{KO/KO} (right) littermate controls. Statistical comparisons were done using a two-tailed t-test. * represents p<0.01. FIG. 13K shows representative images of GFP immunostaining in skin samples of 3-4 week old Pou4f2^{KO(Cre)/WT} (top left) or Pou4f2^{KO(Cre)/KO(Cre)} (right) littermate controls and representative RNA-FISH for GFP in Pou4f2^{KO(Cre)/WT} and Pou4f2^{KO(Cre)/KO(Cre)} littermate controls are displayed below the skin immunostaining images. In FIG. 13H, * represents two-way ANOVA with a Tukey's HSD post-hoc analysis p<0.01. In FIGS. 13J-13K, * represents two-sided t-test p<0.01 Bar graphs in h,j,k show mean+/−s.e.m.

FIGS. 14A-14H show that a postnatal depletion of Pou4f3 results in the loss of subtype specific gene expression in CGRP-α and CGRP-η neurons. CGRP-ε FIG. 14A shows a quantitative RT-PCR analysis using cDNA generated from animals transduced with Luciferase or Pou4f3^{shRNA} expressing AAVs. Error bars represent mean+/−standard error of the mean. Statistical comparisons were done using a paired two-sample t-test. * represents p<0.01. FIG. 14B shows a distribution of the number of UMIs discovered in each population of control sensory neuron. UMI: unique molecular identifier. FIG. 14C shows the distribution of the number of UMIs counted in each each population of shRNA sensory neuron. UMI: unique molecular identifier. FIG. 14D shows t-SNE visualizations of scRNA-seq data for neurons generated from Luciferase^{shRNA} (left) and Pou4f3^{shRNA} littermates DRGs (right). FIG. 14E shows boxplots depicting the fold-change distribution of cell-type specific gene expression in sensory neuron subtypes expressing the highest and lowest levels of Pou4f3 when comparing Luciferase^{shRNA} and Pou4f3^{shRNA} littermate control samples. FIG. 14F shows boxplots depicting the fold-change distribution in a randomized and expression matched control gene set. FIG. 14G shows that control or Pou4f3-depleted mice were exposed to two surfaces with the indicated temperature (x-axis), and the percentage of time spent in the 30° C. chamber over a 5 minute test period. FIG. 14H shows representative images of CGRP immunostaining in skin samples of 1-2 week old Luciferase^{shRNA} (left) or Pou4f3^{shRNA} (right) littermate controls. In FIGS. 14A, 14G, and 14H, mean+/−s.e.m displayed. In FIG. 14A, * represents two-sided t-test p<0.01. In FIG. 14G, * represents two-way ANOVA with a Tukey's HSD post-hoc analysis p<0.05.

FIGS. 15A-15D show subtype restricted TF expression profiles in NGF^{-/-}; Bax^{-/-} cell clusters. FIG. 15A is a heatmap depicting expression of the subtype-restricted TFs in P0 somatosensory subtypes (left) or the clusters from NGF^{-/-}; Bax^{-/-} mutants (right). FIG. 15B shows a single molecule RNA FISH for pairs of subtype restricted TFs in NGF^{-/-}; Bax^{-/-} (top) or littermate NGF^{-/-}; Bax^{-/-} mutants (bottom). FIG. 15C shows the quantification of the RNA-FISH data showing the number of Pou4f3/Shox2 double-positive, Pou4f3/Hopx double-positive, Bcl11a/Hopx double-positive, Neurod1 single-positive or Neurod6 single-positive neurons. FIG. 15D shows a schematized model of gene expression programs as cells traverse development milestones. Transcriptionally unspecialized sensory neurons that emerge from Sox10⁺ and Neurogenin⁺ progenitors co-express multiple TFs which become restricted to select subtype as neurons mature. These TFs are responsible for establishing the transcriptional specializations found in each neuronal subtype. In FIG. 15C, * represents wo-sided t-test p<0.01. TPT: tags per ten thousand.

FIGS. 16A-16B show an assignment of priorities for different somatosensory nodes. The assigned priority levels were color coded for high value target (green), medium value (yellow), and low value (red). The low value was assigned for proprioceptors, which can affect balance.

FIG. 17 shows the assigned numeral codes for each specific node.

FIG. 18 shows tables summarizing the numeral and the color codes for each neural node.

FIG. 19 shows a tables of nodes. To set up a general structure, a list of 165 genes were selected and analyzed for this assignment. The selected genes were individually inserted in to the transcriptome atlas website (kleintools.hms.harvard.edu/tools/springViewer_1_6_dev.html?datasets/Sharma20 19/all). Then, associated nodes were noted down on a full-table with their numeral codes and then further color-coded as described in FIG. 16A. The full-table could be zoomed further for visualization, and this figure shows that magnified portion of the table.

FIG. 20 shows a table sorted to identify the relevant genes. The full table contained genes that were annotated as follows: unspecified genes annotated as ‘unspec’ (unspecified, embryonic) or on multiple nodes annotated as EW (everywhere) or that were either not on the atlas or had no signal as NW (nowhere). These genes were sorted out, which resulted in a 72 gene list. This figure shows a magnified portion of the table. Each green, yellow or red box indicates an individual node and the nodes written in column A approximately show the highly stained node compared to the other nodes in the row for the same gene.

FIG. 21 shows a table sorted to identify the relevant genes with each node labeled with their respective numeral codes.

FIG. 22 shows a graphical representation to show nodal specificity for each gene. The full graph for all the 72 genes (top, can be zoomed) and a magnified portion of the graph (bottom) is shown. The dotted red line is a marker to indicate the bars above which genes are highly specific and thus have only one nodal involvement.

FIG. 23 shows summary tables analyzing genes with only one or two nodal involvement.

FIG. 24 shows a summary table for genes with one nodal involvement and clustered according to their nodes. Some of the genes came out to be false positives such as Gpr3711 but other were true positive (circled in red) and selected for further validation.

FIG. 25 shows the genes with one nodal involvement that were selected for validation.

FIG. 26 shows the genes with two nodal involvement that were selected for validation.

FIG. 27 shows scRNA-seq analysis of all subtypes of DRG sensory neurons from progenitors to adulthood. The CGRP+ subsets are nociceptors, whereas the Somatostatin+ neurons are pruriceptors.

FIG. 28 shows an example identification of two GPCRs with expression profiles restricted to most CGRP+ nociceptors (GPR149) or the large class of adult pruriceptors (HTR1F).

FIG. 29A is a table showing a list of GPCRs with expression restricted to nociceptor or pruriceptor subtypes. The expression patterns of two of these (HTR1F and GPR149) are shown in FIG. 28. This group of GPCRs can be used in certain embodiments to screens for agents that activate GPCRs. In some embodiments, the activation of the GPCR is coupled to the G_{ai/o}-signaling pathway. The agents can be in various embodiments, small molecules, peptides, or antigen binding proteins which specifically bind to and activate GPCR. Concomitantly, the activation of GPCR (which may be coupled to G_{ai/o}-signaling pathway) will inhibit nociceptor and/or pruriceptor subtypes which were identified by the inventors to show restricted expression of said GPCRs.

FIG. 29B is a table showing a list of GPCRs with expression restricted to nociceptor or pruriceptor subtypes. This subgroup of GPCRs can be used in certain embodiments to screens for agents that activate GPCRs. In some embodiments, the activation of the GPCR is coupled to the G_{ai/o}-signaling pathway. The agents can be in various embodiments, small molecules, peptides, or antigen binding proteins which specifically bind to and activate GPCR. Concomitantly, the activation of GPCR (which may be coupled to G_{ai/o}-signaling pathway) will inhibit nociceptor and/or pruriceptor subtypes which were identified by the inventors to show restricted expression of said GPCRs.

FIG. 30 is a panel of RNAScope in situ hybridizations showing that some of the GPCRs found enriched in mouse peptidergic nociceptors are also expressed in human DRG peptidergic nociceptors. The panel includes examples of five GPCRs (GPR149, Adra2c, GPR35, HTR1B and PTGFR) which were found to be enriched in mouse nociceptor subtypes were also expressed in human CGRP+(CALCA) nociceptors. Shown are double fluorescent in situ hybridizations for these five GPCR genes and CALCA using human DRGs. White arrows show examples of double positive neurons.

### DEFINITIONS

Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, A D A M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

The disclosure contemplates that agents for activating the G-Protein Coupled Receptors (GPCR) in nociceptor and/or pruriceptor neurons can include small molecule compounds, peptides, and polypeptides (e.g., antibodies or antibody fragments), and the like. In various embodiments, that GPCRs are coupled to the G_{ai/o}-signaling pathway.

With regard to agents which are chemical compounds, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Michael B. Smith, March's Advanced Organic Chemistry, 7^{th} Edition, John Wiley & Sons, Inc., New York, 2013; Richard C. Larock, Comprehensive Organic Transformations, John Wiley & Sons, Inc., New York, 2018; and Carruthers, Some Modern Methods of Organic Synthesis, 3^{rd} Edition, Cambridge University Press, Cambridge, 1987.

Compounds contemplated herein can comprise one or more asymmetric centers, and thus can exist in various stereoisomeric forms, e.g., enantiomers and/or diastereomers. For example, the compounds contemplated herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, N Y, 1962); and Wilen, S. H., Tables of Resolving Agents and Optical Resolutions p. 268 (E. L. Eliel, Ed., Univ. of Notre Dame Press, Notre Dame, I N 1972). The disclosure additionally contemplates compounds as individual isomers substantially free of other isomers, and alternatively, as mixtures of various isomers.

As used herein, a “subject” means a human or animal. Usually the animal is a vertebrate such as a primate, rodent, domestic animal or game animal. Primates include, for example, chimpanzees, cynomolgus monkeys, spider monkeys, and macaques, e.g., Rhesus. Rodents include, for example, mice, rats, woodchucks, ferrets, rabbits and hamsters. Domestic and game animals include, for example, cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

As used herein, the terms “treat,” “treatment,” “treating,” or “amelioration” refer to therapeutic treatments, e.g., the inhibition of pain perception and/or itch perception, wherein the object is to reverse, alleviate, ameliorate, inhibit, slow down or stop the progression or severity of a condition associated with a disease. The term “treating” includes reducing or alleviating pain and/or itching or the perception thereof. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “small molecule” refers to a organic or inorganic molecule, either natural (i.e., found in nature) or non-natural (i.e., not found in nature), which can include, but is not limited to, a peptide, a peptidomimetic, an amino acid, an amino acid analog, a polynucleotide, a polynucleotide analog, an aptamer, a nucleotide, a nucleotide analog, an organic or inorganic compound (e.g., including heterorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. Examples of “small molecules” that occur in nature include, but are not limited to, taxol, dynemicin, and rapamycin. In certain other preferred embodiments, natural-product-like small molecules are utilized.

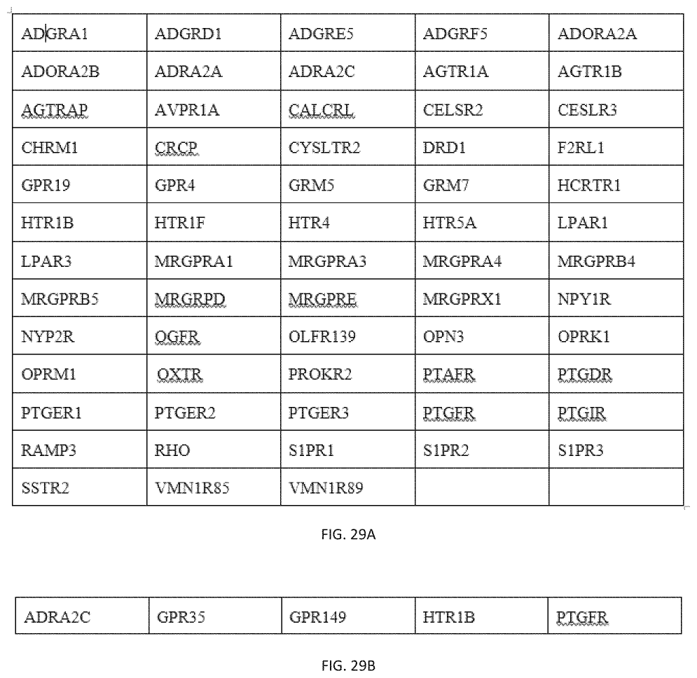

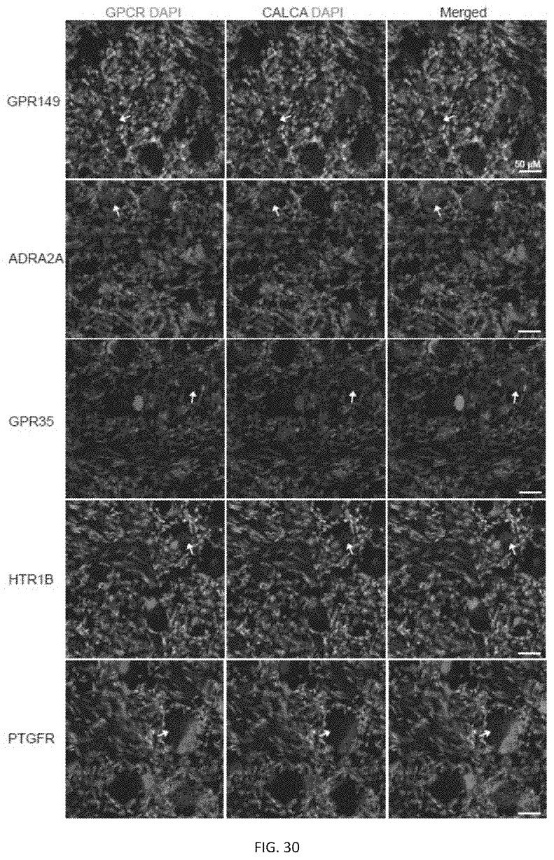

As used herein, a “compound” refers to any chemical, test chemical, drug, new chemical entity (NCE), or other moiety. For example, a compound can be any foreign chemical not normally present in a subject such as mammals including humans. A compound can also be an endogenous chemical that is normally present and synthesized in biological systems, such as mammals including humans. For example, a compound, such as a test compound, such as a drug, can activate GPCRs (such as those identified in FIGS. 29A and 29B) which are specifically expressed in nociceptor and/or pruriceptors subtypes, but not in other sensory neuron subtypes. A compound can be a candidate compound, the ability or capacity of which to activate a nociceptor and/or pruriceptor-specific GPCR would need to be assayed in accordance with the herein methods. The compounds may be provided as plurality of candidate compounds in the form of a library, e.g., a combinatorial library. Such compounds may be further tested using in vitro and/or in vivo approaches (e.g., using animal models) to show their ability to inhibit the perception of pain and/or itch.

The term “derivative” as used herein means any chemical, conservative substitution, or structural modification of an agent. The derivative can improve characteristics of the agent or small molecule such as pharmacodynamics, pharmacokinetics, absorption, distribution, delivery, targeting to a specific receptor, or efficacy. For example, for a small molecule, the derivative can consist essentially of at least one chemical modification to about ten modifications. The derivative can also be the corresponding salt of the agent. The derivative can be the pro-drug of a small molecule as contemplated herein.

An “agent” as used herein is a chemical molecule of synthetic or biological origin. In the context of the present invention, an agent is generally a molecule that can be used in a pharmaceutical composition. Agents may include small molecule compounds, peptides, polypeptides, and antigen binding proteins (including antibodies), and the like. Agents can be a candidate agent, the ability or capacity of which to activate a nociceptor and/or pruriceptor-specific GPCR would need to be assayed in accordance with the herein methods. The agents may be provided as plurality of candidate agents in the form of a library, e.g., a peptide or antibody library. For example, the herein disclosed method may be used to screen and identify a small molecule, peptide, or antibody agent which can activate GPCRs (such as those identified in FIGS. 29A and 29B) which are specifically expressed in nociceptor and/or pruriceptors subtypes, but not in other sensory neuron subtypes. Such agents may be further tested using in vitro and/or in vivo approaches (e.g., using animal models) to show their ability to inhibit the perception of pain and/or itch.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. The term “pharmaceutically acceptable carrier” excludes tissue culture media. Each carrier must be “acceptable” in the sense of being compatible with the other ingredients of the formulation, for example the carrier does not decrease the impact of the agent on the treatment. In other words, a carrier is pharmaceutically inert. The terms “physiologically tolerable carriers” and “biocompatible delivery vehicles” are used interchangeably. Non-limiting examples of pharmaceutical carriers include particle or polymer-based vehicles such as nanoparticles, microparticles, polymer microspheres, or polymer-drug conjugates.

The term “effective amount” is used interchangeably with the term “therapeutically effective amount” or “amount sufficient” and refers to the amount of at least one activator of a GPCR, or a pharmaceutical composition thereof, at dosages and for periods of time necessary to achieve the desired therapeutic result, for example, to “attenuate,” reduce or stop at least one symptom of pain and/or chronic itch. For example, an effective amount using the methods as disclosed herein would be considered as the amount sufficient to reduce one or more symptoms of pain and/or chronic itch by at least 10%, as compared to the level of pain and/or chronic itch in the absence of the compound or agent. In other embodiments, an effective amount using the methods as disclosed herein would be considered as the amount sufficient to reduce one or more symptoms of pain and/or chronic itch by at least 5%, or by at least 10%, or by at least 15%, or by at least 20%, or by at least 25%, or by at least 30%, or by at least 35%, or by at least 40%, or by at least 45%, or by at least 50%, or by at least 55%, or by at least 60%, or by at least 65%, or by at least 70%, or by at least 75%, or by at least 80%, or by at least 85%, or by at least 90%, or by at least 95%, or up to 100% as compared to the level of pain and/or chronic itch in the absence of the compound or agent. An effective amount as used herein would also include an amount sufficient to prevent or delay the development of such a symptom, alter the course of a symptom disease (for example but not limited to, slow the progression of a symptom of the disease), or reverse a symptom of the disease in a subject suffering from pain and/or chronic itch. Accordingly, the term “effective amount” or “therapeutically effective amount” as used herein refers to the amount of therapeutic agent of a pharmaceutical composition to alleviate at least one symptom of a disease. Stated another way, “therapeutically effective amount” of an activator of a GPCR as disclosed herein is the amount of an agonist which exerts a beneficial effect on, for example, the symptoms of the disease. The dosage administered, as single or multiple doses, to an individual will vary depending upon a variety of factors, including pharmacokinetic properties of the inhibitor, the route of administration, conditions and characteristics (sex, age, body weight, health, size) of subjects, extent of symptoms, concurrent treatments, frequency of treatment and the effect desired. A therapeutically effective amount is also one in which any toxic or detrimental effects of the therapeutic agent are outweighed by the therapeutically beneficial effects. The effective amount in each individual case can be determined empirically by a skilled artisan according to established methods in the art and without undue experimentation. In general, the phrases “therapeutically-effective” and “effective for the treatment, prevention, or inhibition”, are intended to qualify agonist as disclosed herein which will achieve the goal of reduction in the severity of a pain and/or chronic itch or at one related symptom thereof. In certain embodiments, the term “effective amount” means a dosage sufficient to produce a desired result. The desired result can be subjective or objective changes in the biological activity of a GPCR, especially signal transduction. Effective amounts of the GPCR polypeptide or composition, which may also include a functional derivative thereof, are from about 0.01 micrograms to about 100 mg/kg body weight, and preferably from about 10 micrograms to about 50 mg/kg body weight, such 0.05, 0.07, 0.09, 0.1, 0.5, 0.7, 0.9, 1, 2, 5, 10, 20, 25, 30, 40, 45, or 50 mg/kg.

In jurisdictions that forbid the patenting of methods that are practiced on the human body, the meaning of “administering” of a composition to a human subject shall be restricted to prescribing a controlled substance that a human subject will self-administer by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

As used herein, the term “administer” refers to the placement of a composition into a subject by a method or route which results in at least partial localization of the composition at a desired site such that desired effect is produced. A compound or composition described herein can be administered by any appropriate route known in the art including, but not limited to, oral or parenteral routes, including intravenous, intramuscular, subcutaneous, transdermal, airway (aerosol), pulmonary, nasal, rectal, and topical (including buccal and sublingual) administration.

The phrases “parenteral administration” and “administered parenterally” as used herein means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection, infusion and other injection or infusion techniques, without limitation. Without limitations, oral administration can be in the form of solutions, suspensions, tablets, pills, capsules, sustained-release formulations, oral rinses, powders and the like.

As used herein, the term “modulates” refers to an effect including increasing or decreasing a given parameter as those terms are defined herein.

As used herein, the term “contacting” when used in reference to a cell or organ, encompasses both introducing or administering an agent (small molecule, peptide, or antibody anti-pain or anti-itch agent) to the cell, tissue, or organ in a manner that permits physical contact of the cell with the agent.

The term “statistically significant” or “significantly” refers to statistical significance and generally means a two-standard deviation (2SD) or greater difference.

As used herein the term “comprising” or “comprises” is used in reference to compositions, methods, and respective component(s) thereof, that are essential to the method or composition, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

As used herein, the term “G-protein” is any member of the superfamily of signal transducing guanine nucleotide binding proteins.

As used herein, the term “G-protein-coupled receptor” is any member of a superfamily of receptors that mediates signal transduction by coupling with a G protein. Examples of such receptors include, but are not limited to: CC chemokine receptor 5 (CCR5), CXC chemokine receptor (CXCR4) cholecystokinin type A receptor (CCKAR), adenosine receptors, somatostatin receptors, dopamine receptors, muscarinic cholinergic receptors, alpha-adrenergic receptors, beta-adrenergic receptors, opiate receptors, cannabinoid receptors, growth hormone releasing factor, glucagon, cAMP receptors, serotonin receptors (5-HT), histamine H2 receptors, thrombin receptors, kinin receptors, follicle stimulating hormone receptors, opsins and rhodopsins, odorant receptors, cytomegalovirus GPCRs, histamine H2 receptors, octopanmine receptors, N-formyl receptors, anaphylatoxin receptors, thromboxane receptors, IL-8 receptors, platelet activating factor receptors, endothelin receptors, bombesin gastrin releasing peptide receptor, neuromedin B preferring bombesin receptors, vasoactive intestinal peptide receptors, neurotensin receptors, bradykinin receptors, thyrotropin-releasing hormone receptors, substance P receptors, neuromedin K receptors, renal angiotensin II type I receptors, mas oncogene (angiotensin) receptors lutropin-choriogonadotropin receptors, thyrotropin receptors, follicle stimulating hormone receptors, cannabinoid receptors, glucocorticoid-induced receptors, endothelial cell GPCRs, testis GPCRs, and thoracic aorta GPCRs, and homologs thereof having a homology of at least 80% with at least one of transmembrane domains 1-7, as described herein. See, e.g., Probst et al, DNA and Cell Biology 11: 1-20 (1992), which is entirely incorporated herein by reference. The term further encompasses subtypes of the named receptors, and mutants and homologs thereof, along with the DNA sequences encoding the same. Other examples which are disclosed in FIG. 29A and which were shown to be specifically expressed in nociceptros and/or pruriceptors, include: ADGRA1, ADGRD1, ADGRE5, ADGRF5, ADORA2A, ADORA2B, ADRA2A, ADRA2C, AGTR1A, AGTR1B, AGTRAP, AVPR1A, CALCRL, CELSR2, CESLR3, CHRM1, CRCP, CYSLTR2, DRD1, F2RL1, F2RL2, FZD3, FZD5, FZD8, GALR1, GHSR, GPR35, GPR149, GPR156, GPR173, GPR174, GPR19, GPR4, GRM5, GRM7, HCRTR1, HTR1B, HTR1F, HTR4, HTR5A, LPAR1, LPAR3, MRGPRA1, MRGPRA3, MRGPRA4, MRGPRB4, MRGPRB5, MRGRPD, MRGPRE, MRGPRX1, NPY1R, NYP2R, OGFR, OLFR139, OPN3, OPRK1, OPRM1, OXTR, PROKR2, PTAFR, PTGDR, PTGER1, PTGER2, PTGER3, PTGFR, PTGIR, RAMP3, RHO, S1PR1, S1PR2, S1PR3, SSTR2, VMN1R85, and VMN1R89.

As used herein, GPCR “ligands” refers to biological molecules that bind GPCRs in vitro, in situ or in vivo, and may include small molecule compounds, peptides, and antibodies and the like (or other agents disclosed herein).

As used herein, “nociceptors” are specialized somatorsensory neurons located in the peripheral nervous system which are activated by potentially noxious stimuli, such as thermal, mechanical, or chemical stimuli, which are transmitted as a pain signal to the central nervous system. The process of sensing pain is called nociception. Nociceptive pain can be classified according to the tissue in which the nociceptor activation occurred: superficual somatic (e.g., skin), deep somatic (e.g., ligaments/tendons/bones/muscles) or visceral (internal organs).

As used herein, “pruriceptors” are specialized somatosensory neurons located in the peripheral nervous system which are activated to perceive itching sensations.

As used herein, reference to “pruritus” is defined as an unpleasant sensation of the skin that provokes the urge to scratch. It is a characteristic feature of many skin diseases and an unusual sign of some systemic diseases. Pruritus may be localized or generalized and can occur as an acute or chronic condition. Itching lasting more than 6 weeks is termed “chronic pruritus.” Itching can be intractable and incapacitating, as well as a diagnostic and therapeutic challenge.

As used herein, a dorsal root ganglia (or “DRG”) refers to the cluster (or ganglion) of neurons in a dorsal root of a spinal nerve which includes cell bodies of sensory neurons which relay sensory information (e.g., pain) from the periphery (e.g., the skin) to the spinal cord.

As used herein, the term “signals” refer to internal and external factors that control changes in cell structure and function. They are chemical or physical in nature.

As used herein, the term “signaling” in reference to a “signal transduction protein” refers to proteins that are activated or otherwise affected by ligand binding to a membrane receptor protein or some other stimulus.

All references cited herein, including journal articles or abstracts, published or corresponding U.S. or foreign patent applications, issued U.S. or foreign patents, or any other references, are entirely incorporated by reference herein, including all data, tables, figures, and text presented in the cited references. Additionally, the contents of the references cited within the references cited herein are also entirely incorporated by reference.

### DETAILED DESCRIPTION

The somatosensory system is a part of the sensory nervous system. The somatosensory system is a complex system of sensory neurons (i.e., somatosensory neurons) and neural pathways that responds to changes at the surface or inside the body. The axons as afferent nerve fibers of sensory neurons connect with, or respond to, various receptor cells. These sensory receptor cells are activated by different stimuli such as heat and nociception, giving a functional name to the responding sensory neuron, such as a thermoreceptor which carries information about temperature changes. Other sensory neuron subtypes include mechanoreceptors, chemoreceptors, and nociceptors which send signals along a sensory nerve to the spinal cord where they may be processed by other sensory neurons and then relayed to the brain for further processing. Sensory receptors are found all over the body including, the skin, epithelial, tissues, muscles, bones and joints, internal organs, and the cardiovascular system.

The present disclosure is based, in part, on the inventors' discovery that certain G-protein coupled receptors (GPCRs), and in particular, those which are coupled to the G_{ai/o}-signaling pathway, are restricted in expression to certain nociceptor and pruriceptor subtypes, but not proprioceptors, mechanoreceptors, or other sensory neurons subtypes that are not involved in conveying pain and/or itch sensations. In particular, the present inventors herein discovered and describe at least six transcriptionally distinct cellular subtypes of nociceptors and at least two transcriptionally distinct cellular subtypes of pruriceptors, and revealed a set of new therapeutic GPCR targets showing restricted expression in said nociceptor and/or pruriceptor subtypes which may be selectively activated to inhibit pain and/or itch processing pathways, without affecting other sensory pathways (e.g., sensing through proprioceptors or mechanoceptors).

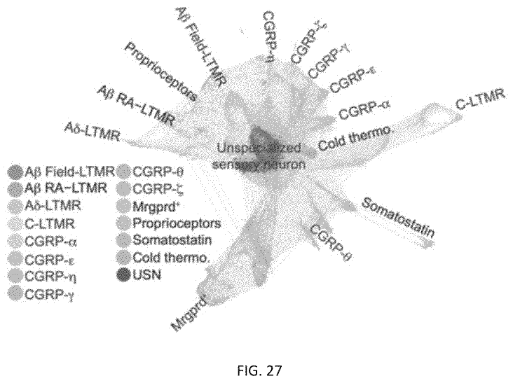

The present inventors show in FIG. 27 scRNA-seq analysis of all subtypes of DRG sensory neurons from progenitors to adulthood. The CGRP+ subsets are nociceptors (including CGRP-Theta (CGRP-θ), CGRP-Eta (CGRP-η), CGRP-Zeta (CGRP-ζ), CGRP-Gamma (CGRP-γ), CGRP-Epsilon (CGRP-ε), and CGRP-Alpha (CGRP-α)), whereas the Somatostatin+ neurons are pruriceptors (including Somatostatin (Sst)).

The inventors describe in FIG. 29A a set of the discovered GPCR targets having expression restricted to nociceptor and/or pruriceptor subtypes, such as nociceptor subtypes CGRP-Theta (CGRP-θ), CGRP-Eta (CGRP-η), CGRP-Zeta (CGRP-ζ), CGRP-Gamma (CGRP-γ), CGRP-Epsilon (CGRP-ε), and CGRP-Alpha (CGRP-α)) sensory neurons and pruriceptor subtypes somatostatin+ sensory neurons. In particular, FIG. 29A shows the following identified GPCRs: ADGRA1, ADGRD1, ADGRE5, ADGRF5, ADORA2A, ADORA2B, ADRA2A, ADRA2C, AGTR1A, AGTR1B, AGTRAP, AVPR1A, CALCRL, CELSR2, CESLR3, CHRM1, CRCP, CYSLTR2, DRD1, F2RL1, F2RL2, FZD3, FZD5, FZD8, GALR1, GHSR, GPR35, GPR149, GPR156, GPR173, GPR174, GPR19, GPR4, GRM5, GRM7, HCRTR1, HTR1B, HTR1F, HTR4, HTR5A, LPAR1, LPAR3, MRGPRA1, MRGPRA3, MRGPRA4, MRGPRB4, MRGPRB5, MRGRPD, MRGPRE, MRGPRX1, NPY1R, NYP2R, OGFR, OLFR139, OPN3, OPRK1, OPRM1, OXTR, PROKR2, PTAFR, PTGDR, PTGER1, PTGER2, PTGER3, PTGFR, PTGIR, RAMP3, RHO, S1PR1, S1PR2, S1PR3, SSTR2, VMN1R85, and VMN1R89. This group of GPCRs can be used in certain embodiments to screens for agents that activate GPCRs. In some embodiments, the activation of the GPCR is coupled to the G_{ai/o}-signaling pathway. The agents can be in various embodiments, small molecules, peptides, or antigen binding proteins which specifically bind to and activate GPCR. Concomitantly, the activation of GPCR (which may be coupled to G_{ai/o}-signaling pathway) will inhibit nociceptor and/or pruriceptor subtypes which were identified by the inventors to show restricted expression of said GPCRs.

The inventors further show in FIG. 29B a set of the herein discovered GPCR targets having expression restricted to nociceptor and/or pruriceptor subtypes, such as nociceptor subtypes CGRP-Theta (CGRP-θ), CGRP-Eta (CGRP-η), CGRP-Zeta (CGRP-ζ), CGRP-Gamma (CGRP-γ), CGRP-Epsilon (CGRP-ε), and CGRP-Alpha (CGRP-α)) sensory neurons and pruriceptor subtypes somatostatin+ sensory neurons. In particular, FIG. 29B shows the identification of GPCRs of ADRA2C, GPR35, GPR149, HTR1B, and PTGFR. This subgroup of GPCRs can be used in certain embodiments to screens for agents that activate GPCRs. In some embodiments, the activation of the GPCR is coupled to the G_{ai/o}-signaling pathway. The agents can be in various embodiments, small molecules, peptides, or antigen binding proteins which specifically bind to and activate GPCR. Concomitantly, the activation of GPCR (which may be coupled to G_{ai/o}-signaling pathway) will inhibit nociceptor and/or pruriceptor subtypes which were identified by the inventors to show restricted expression of said GPCRs.

Through their work, the inventors determined that an ideal GPCR to be used as a target for an agent (e.g., small molecule compound, peptide, or antigen binding protein) that is useful for treating pain and/or itch may have one or more of the following properties, and in some embodiments, all of the following properties: (1) the GPCR is highly expressed in nociceptors, pruriceptors, or combinations of both; (2) the GPCR is coupled to the G_{ai/o} signaling pathway; (3) the GPCR exhibits a conserved pattern of expression between rodent and human DRGs; (4) the GPCR is expressed at low levels in other sensory neuron subtypes (e.g., mechanoreceptors, proprioceptors, or other peripheral sensory neurons that convey sensations such as temperature, pressure, and limb movement or position, excluding pain and/or itch sensations), as well as low levels of expression in the peripheral tissues and/or the brain; and (5) activation of the GPCR attenuates pain or itch perception, in particular, where the GPCR is coupled to the G_{ai/o} signaling pathway.

Accordingly, the present disclosure provides a newly conceived of platform for identifying novel pain and/or pruritus-inhibiting agents (e.g., small molecule compounds, peptides, antigen binding proteins (e.g., antibodies or antibody fragments) that are efficacious, safe, and non-addictive alternatives for pain and pruritus management in place of (or in some embodiments, in combination with) “first line” treatments, such as gabapentin, pregabalin, and opioids. In another aspect, the present disclosure provides methods for treating pain and/or pruritus with pain and/or pruritus-inhibiting agents (e.g., small molecule compounds, peptides, antigen binding proteins (e.g., antibodies or antibody fragments) that are efficacious, safe, and non-addictive alternatives for pain and pruritus management in place of (or in some embodiments, in combination with) “first line” treatments, such as gabapentin, pregabalin, and opioids.

Thus, in various aspects, the present disclosure provides (1) a screening platform for identifying novel pain and/or pruritus-inhibiting agents (e.g., small molecule compounds, peptides, antigen binding proteins (e.g., antibodies or antibody fragments) that is based on the inventors' discovery that certain GPCRs are restricted in expression to one or more subsets of nociceptors and pruriceptors, but not proprioceptors or other sensory neurons subtypes not involved in pain and/or itch detection. In certain embodiments, that GPCRs are coupled to the G_{ai/o} signaling pathway. The present disclosure further provides (2) identified pain and/or pruritus-inhibiting agents (e.g., small molecule compounds, peptides, antigen binding proteins (e.g., antibodies or antibody) which (i) activate certain GPCRs (e.g., those of FIG. 29A and FIG. 29B) which show restricted expression in one or more subsets of nociceptors and pruriceptors (e.g., sensory neurons identified as CGRP-Theta (CGRP-θ), CGRP-Eta (CGRP-η), CGRP-Zeta (CGRP-ζ), CGRP-Gamma (CGRP-γ), CGRP-Epsilon (CGRP-ε), and CGRP-Alpha (CGRP-α)) nociceptor sensory neurons and somatostatin+ sensory pruriceptor neurons), but not proprioceptors or other sensory neurons subtypes not involved in pain and/or itch perception. In some embodiments, the agents can be known GPCR agonists which are tested and confirmed to activate one or more of the nociceptor-specific or pruripotent-specific GPCRs identified by the inventors (e.g., those of FIG. 29A and FIG. 29B). In other embodiments, the agents can be previously known agents, but which were not previously known to bind to and activate one or more of the nociceptor-specific or pruripotent-specific GPCRs identified by the inventors (e.g., those of FIG. 29A and FIG. 29B). In still other embodiments, the agents can be novel agents, but which did not previously exist in the art and which were or can be shown to bind to and activate one or more of the nociceptor-specific or pruripotent-specific GPCRs identified by the inventors (e.g., those of FIG. 29A and FIG. 29B).

Accordingly, in various embodiments, the disclosure provides methods for screening for agents from a plurality of candidate agents (a library of small molecules, peptides, or antigen binding proteins (e.g., antibodies), wherein said agents are pain and/or pruritus-inhibiting agents (e.g., small molecule compounds, peptides, antigen binding proteins (e.g., antibodies or antibody fragments) that bind to and activate one or more GPCRs which are restricted in expression to one or more subsets of nociceptors and pruriceptors but which are not expressed in proprioceptors or other sensory neuron subtypes not involved in pain and/or itch detection. In certain embodiments, that GPCRs are coupled to the G_{ai/o} signaling pathway. In various embodiments, the disclosure also provides methods of testing and confirming whether a given GPCR is coupled to the G_{ai/o} signaling pathway.

In still other embodiments, the present disclosure provides libraries of candidate agents, e.g., small molecule libraries, peptide libraries, antibody libraries, etc. which may be screened using the methods disclosed herein to assay for binding to and activating one or more GPCRs (e.g., the GPCRs of FIG. 29A or 29B) which are restricted in expression to one or more subsets of nociceptors and pruriceptors but which are not expressed in proprioceptors or other sensory neuron subtypes not involved in pain and/or itch detection.

Libraries of candidate agents may be constructed using any methods known in the art, or obtained from any suitable source, so long as they are suitable for screening using the methods disclosed herein, e.g., Examples 1-3. For examples, combinatorial peptide or polypeptide libraries of GPCR polypeptide modulators are described in U.S. Pat. Nos. 10,745,456, 7,232,659, the contents of which are incorporated by reference. In addition, agents that bind and activate GPCRs are described U.S. Pat. No. 10,590,196 (Antibodies targeting G-protein coupled receptor and methods of use), U.S. Pat. No. 10,358,416 (Substituted pyrrolidines as G-protein coupled receptor 43 agonists), and U.S. Pat. No. 8,193,359 (G-protein coupled receptor agonists), each of which are incorporated herein by reference in their entireties. These agents and derivatives obtained thereof may be screened using the methods disclosed herein to identify agents which activate the nociceptor and/or pruriceptor-expressed GPCRs, e.g., those described in FIGS. 29A and 29B.

In yet other embodiments, the present disclosure provides nucleic acid molecules encoding the nociceptor and/or pruriceptor-specific GPCRs (including those which are coupled to the G_{ai/o} signaling pathway), and to cloning and/or expression vectors comprising said nucleic acid molecules encoding the nociceptor and/or pruriceptor-specific GPCRs. Further, the disclosure provides for cells comprising said cloning and/or expression vector comprising said nucleic acid molecules encoding the nociceptor and/or pruriceptor-specific GPCRs.

In various embodiments, the disclosure also provides for various reagents, biochemical assays, etc. capable of detecting when a particular candidate agent binds to and/or activates a nociceptor and/or pruriceptor-specific GPCR (e.g., one or more of those GPCRs of FIG. 29A or 29B).

In still further embodiments, the disclosure also provides for various reagents, biochemical assays, etc. capable of detecting when a particular candidate agent binds to and/or activates a nociceptor and/or pruriceptor-specific GPCR (e.g., one or more of those GPCRs of FIG. 29A or 29B). Such reagents and/or biochemical assays may be used in connection with in vitro and/or in vivo assays.

In yet further embodiments, the disclosure also provides for various animal models capable of detecting when a particular candidate agent binds to and/or activates a nociceptor and/or pruriceptor-specific GPCR (e.g., one or more of those GPCRs of FIG. 29A or 29B) and further, whether said candidate agents result in the inhibition of pain and/or itch perception in the animal model.

In one aspect, provided herein is a method of treating pain and/or a pruritus comprising administering a therapeutically effective amount of an agent that activates a G_{ai/o}-coupled G-Protein Coupled Receptor (GPCR) that is selectively expressed in the nociceptor and/or pruriceptor neuron subtypes of the somatosensory neurons.

In various embodiments, the disclose method results of the activation of the G_{ai/o}-coupled G-Protein Coupled Receptor (GPCR). The activation of the G_{ai/o}-coupled GPCR results in the blocking and/or reduction of pain and/or itch signaling by nociceptor and/or pruriceptor neurons which express the G_{ai/o}-coupled GPCR.

In various embodiments, the blocking and/or reduction of pain and/or itch signaling by nociceptor and/or pruriceptor neurons which express the G_{ai/o}-coupled GPCR is selective because the particular target G_{ai/o}-coupled GPCR is not expressed or is expressed at low levels (or is not detectable) in other somatosensory neurons that are not nociceptors or pruriceptors.

In some embodiments, the agent binds and activates a G_{ai/o}-coupled G-Protein Coupled Receptor (GPCR) that is selected from the group consisting of: ADGRA1, ADGRD1, ADGRE5, ADGRF5, ADORA2A, ADORA2B, ADRA2A, ADRA2C, AGTR1A, AGTR1B, AGTRAP, AVPR1A, CALCRL, CELSR2, CESLR3, CHRM1, CRCP, CYSLTR2, DRD1, F2RL1, F2RL2, FZD3, FZD5, FZD8, GALR1, GHSR, GPR35, GPR149, GPR156, GPR173, GPR174, GPR19, GPR4, GRM5, GRM7, HCRTR1, HTR1B, HTR1F, HTR4, HTR5A, LPAR1, LPAR3, MRGPRA1, MRGPRA3, MRGPRA4, MRGPRB4, MRGPRB5, MRGRPD, MRGPRE, MRGPRX1, NPY1R, NYP2R, OGFR, OLFR139, OPN3, OPRK1, OPRM1, OXTR, PROKR2, PTAFR, PTGDR, PTGER1, PTGER2, PTGER3, PTGFR, PTGIR, RAMP3, RHO, S1PR1, S1PR2, S1PR3, SSTR2, VMN1R85, and VMN1R89.

In various embodiments, that agent can be identified by performing a high throughput compound screen for molecules that activate the G_{ai/o}-coupled G-Protein Coupled Receptor (GPCR).

In other embodiments, the agent can be a known ligand of a G_{ai/o}-coupled G-Protein Coupled Receptor (GPCR).

In still other embodiments, the activation of the G_{ai/o}-coupled G-Protein Coupled Receptor (GPCR) causes downstream activation of G-protein coupled inwardly rectifying potassium channels (GIRKs). And, the activation of the GIRKS can cause silencing of the neuronal activity of the nociceptor and/or pruriceptor neuron subtypes.

In another aspect, the disclosure provides a method of treating pain and/or a pruritus comprising administering a therapeutically effective amount of an agent that activates a G_{ai/o}-coupled G-Protein Coupled Receptor (GPCR) that is selectively expressed in the nociceptor and/or pruriceptor neuron subtypes of the somatosensory neurons.

In various embodiments, the agent that activates a G_{ai/o}-coupled G-Protein Coupled Receptor (GPCR) results in a reduction or blocking of the pain and/or itch signaling by the nociceptor and/or pruriceptor neuron.

In some embodiments, the G_{ai/o}-coupled G-Protein Coupled Receptor (GPCR) is selectively expressed in the nociceptor and/or pruriceptor neuron subtypes of the somatosensory neurons because it is not expressed or expressed at low levels in other somatosensory neuron subtypes, peripheral tissues, and/or brain.

In some embodiments, the G_{ai/o}-coupled G-Protein Coupled Receptor (GPCR) is selectively expressed in the nociceptor and/or pruriceptor neuron subtypes of the somatosensory neurons, but not detectable in other somatosensory neuron subtypes, peripheral tissues, and/or brain.

In other embodiments, the G_{ai/o}-coupled G-Protein Coupled Receptor (GPCR) is selected from the group consisting of: ADGRA1, ADGRD1, ADGRE5, ADGRF5, ADORA2A, ADORA2B, ADRA2A, ADRA2C, AGTR1A, AGTR1B, AGTRAP, AVPR1A, CALCRL, CELSR2, CESLR3, CHRM1, CRCP, CYSLTR2, DRD1, F2RL1, F2RL2, FZD3, FZD5, FZD8, GALR1, GHSR, GPR35, GPR149, GPR156, GPR173, GPR174, GPR19, GPR4, GRM5, GRM7, HCRTR1, HTR1B, HTR1F, HTR4, HTR5A, LPAR1, LPAR3, MRGPRA1, MRGPRA3, MRGPRA4, MRGPRB4, MRGPRB5, MRGRPD, MRGPRE, MRGPRX1, NPY1R, NYP2R, OGFR, OLFR139, OPN3, OPRK1, OPRM1, OXTR, PROKR2, PTAFR, PTGDR, PTGER1, PTGER2, PTGER3, PTGFR, PTGIR, RAMP3, RHO, S1PR1, S1PR2, S1PR3, SSTR2, VMN1R85, and VMN1R89.

In certain embodiments, the activation of the G_{ai/o}-coupled G-Protein Coupled Receptor (GPCR) causes silencing of neuronal activity of the nociceptor and/or pruriceptor neuron subtypes.

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1: The Emergence of Transcriptional Identity in Somatosensory Neurons

### Abstract

Over a dozen morphologically and physiologically distinct primary somatosensory neuron subtypes report salient features of internal and external environments¹⁻⁴. How specialized gene expression programs emerge during development to endow somatosensory neuron subtypes with their unique properties is unclear. To assess the developmental progression of transcriptional maturation of each principal somatosensory neuron subtype, a transcriptomic atlas of cells traversing the primary somatosensory neuron lineage was generated. It was found that somatosensory neurogenesis gives rise to neurons in a transcriptionally unspecialized state, characterized by co-expression of transcription factors (TFs) that become restricted to select subtypes as development proceeds. Single cell transcriptomic analyses of sensory neurons from mutant mice lacking TFs suggest that these broad-to-restricted TFs coordinate subtype-specific gene expression programs in the subtypes where their expression is maintained. Additionally, a role was defined for neuronal targets for TF expression as disruption of the prototypic target-derived neurotrophic factor NGF leads to aberrant subtype-restricted patterns of TF expression. These findings support a model in which cues emanating from intermediate and final target fields promote neuronal diversification in part by transitioning cells from a transcriptionally unspecialized state to transcriptionally distinct subtypes through modulating selection of subtype-restricted TFs.

Decades of analyses have revealed more than a dozen functionally distinct somatosensory neuron subtypes of the dorsal root ganglia (DRG) that collectively enable detection of a broad range of salient features of the external world¹⁻⁴. A fundamental question in sensory and developmental biology is how somatosensory neuron subtypes acquire their characteristic physiological, morphological, and synaptic properties during development, enabling animals to detect and respond to innocuous and noxious thermal, chemical, and mechanical stimuli. Classical studies of embryonic development indicate that migrating multipotent neural crest progenitors, originating from the dorsal neural tube, populate nascent DRGs⁵. During ganglia formation, dedicated progenitors that express either Neurog1 (neurogenin-1) or Neurog2 (neurogenin-2) are proposed to give rise to distinct somatosensory neuron subtypes⁶, which then innervate peripheral target fields where they form morphologically distinct axonal ending types¹. Current models of somatosensory neuron development have primarily been inferred from studies analyzing changes in expression of individual genes or axonal ending types in loss-of-function models^{1,7,8}. Here, enome-wide transcriptomic analyses were used, coupled with molecular genetic approaches to define transcriptional mechanisms of somatosensory neuron subtype diversification.

scRNA-Seq of Somatosensory Neurons

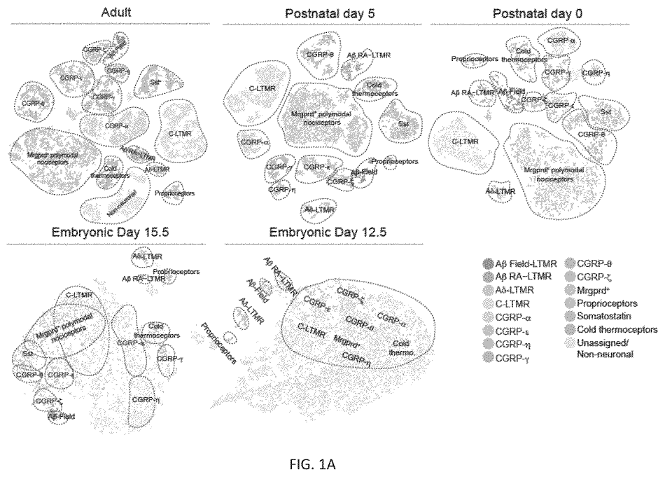

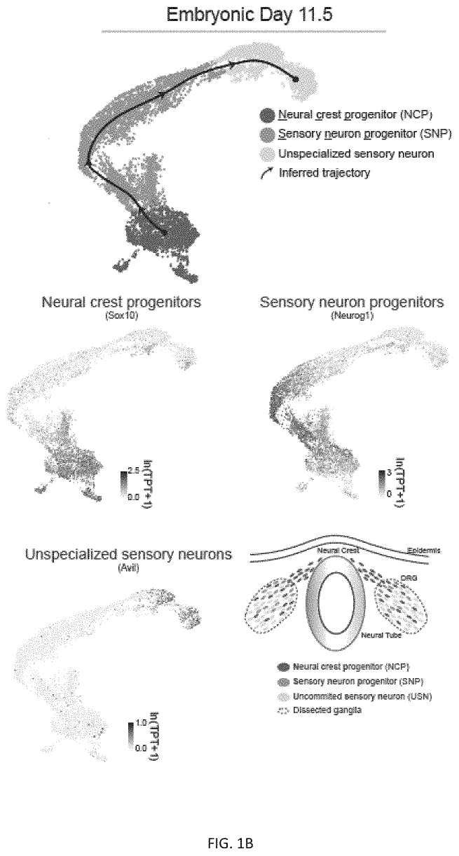

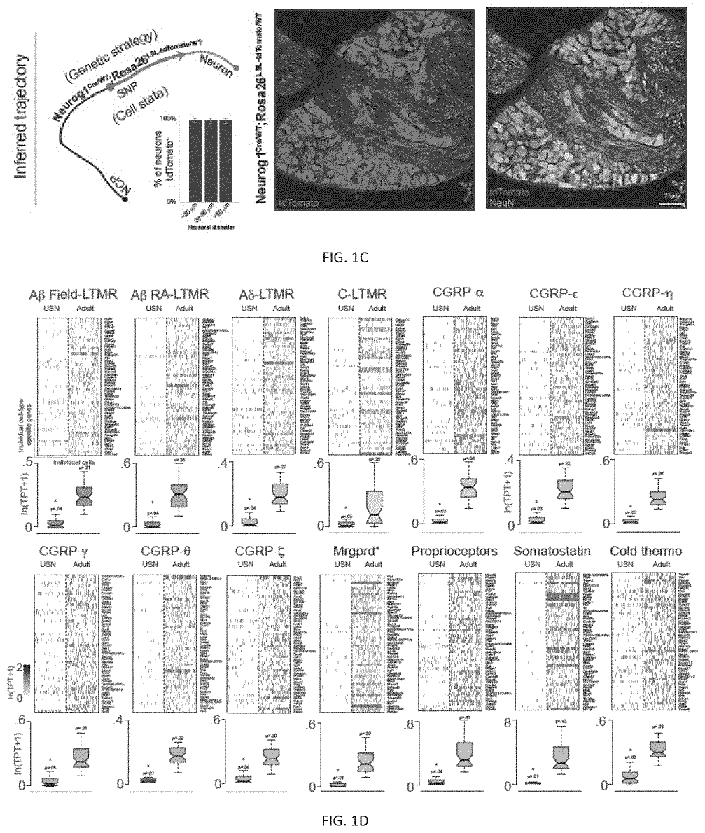

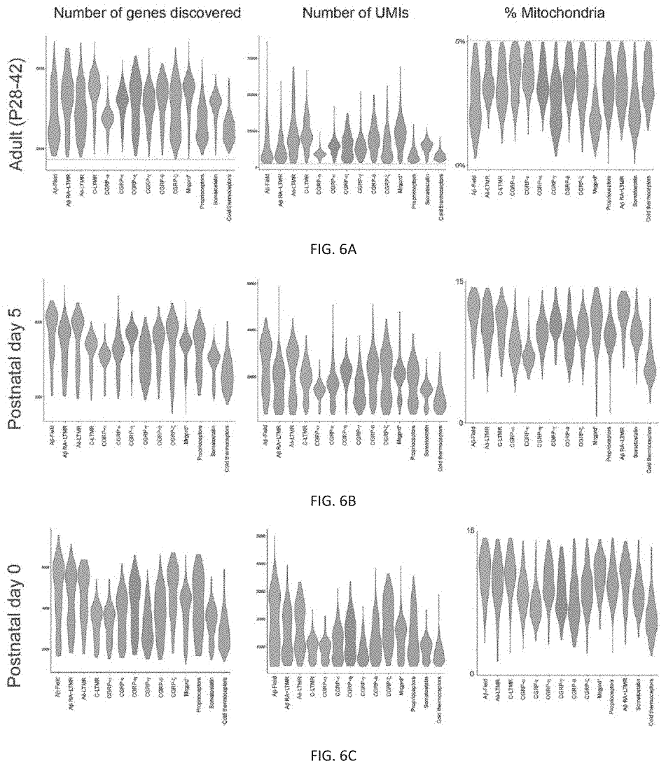

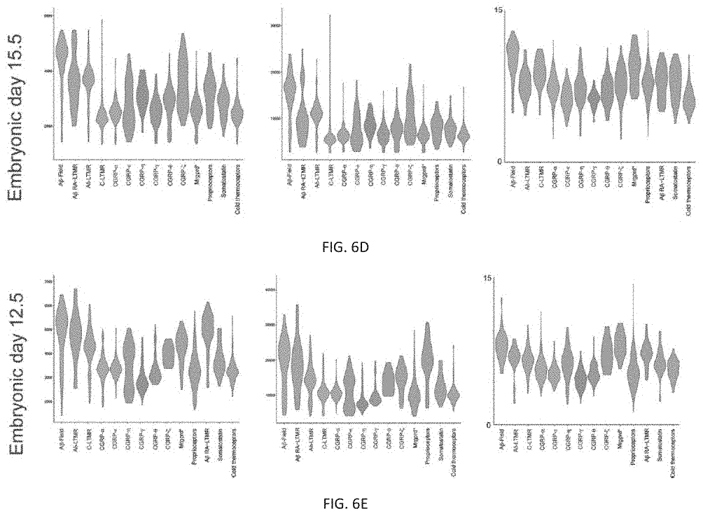

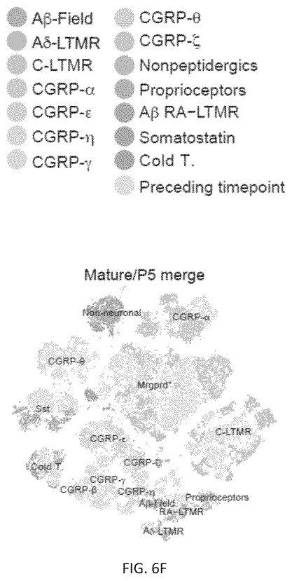

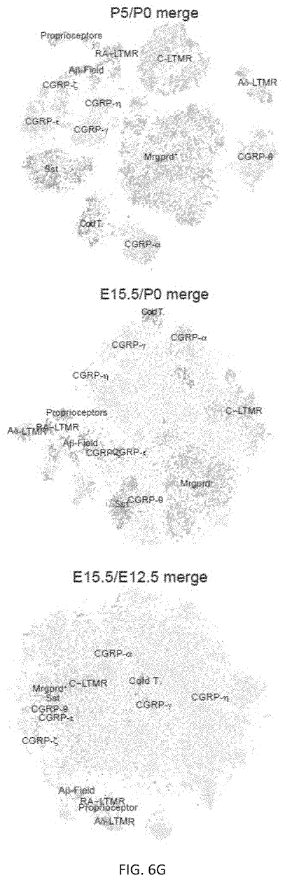

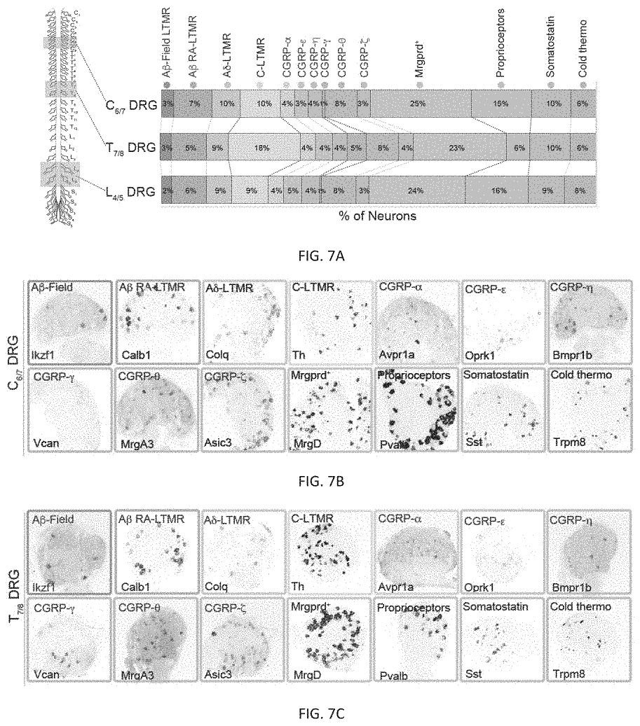

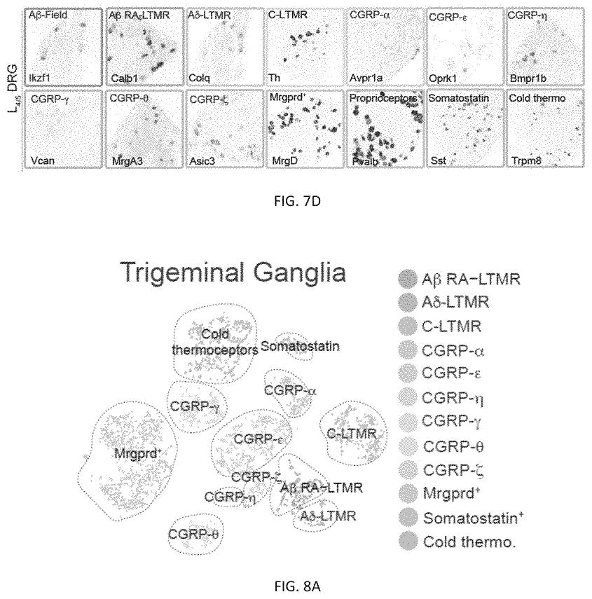

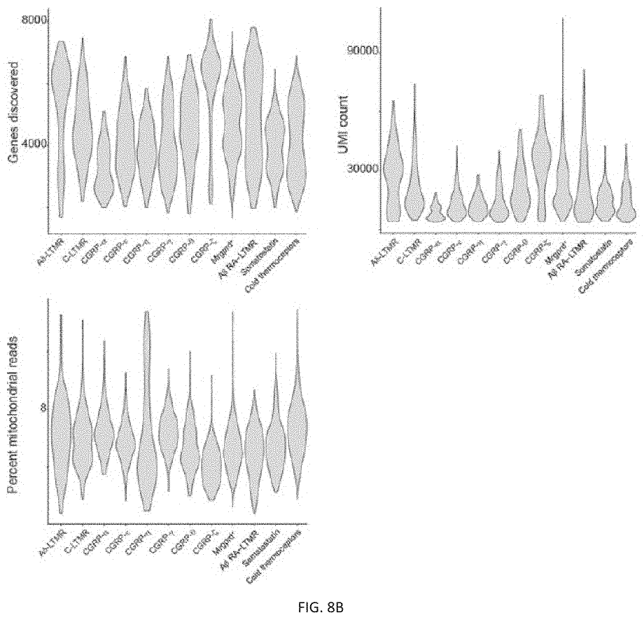

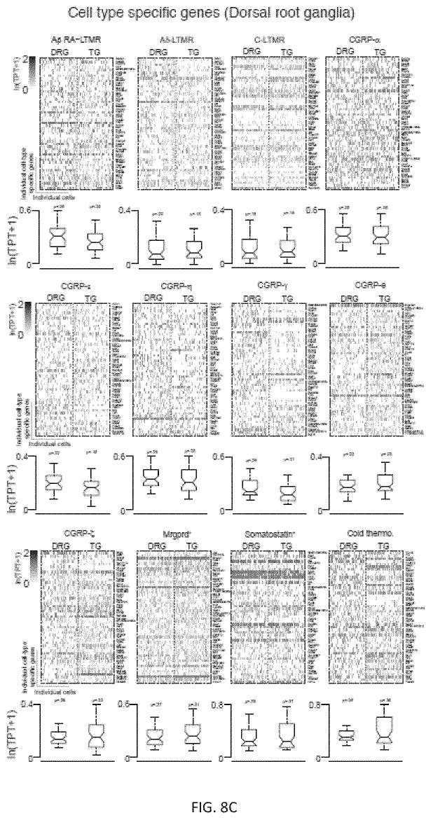

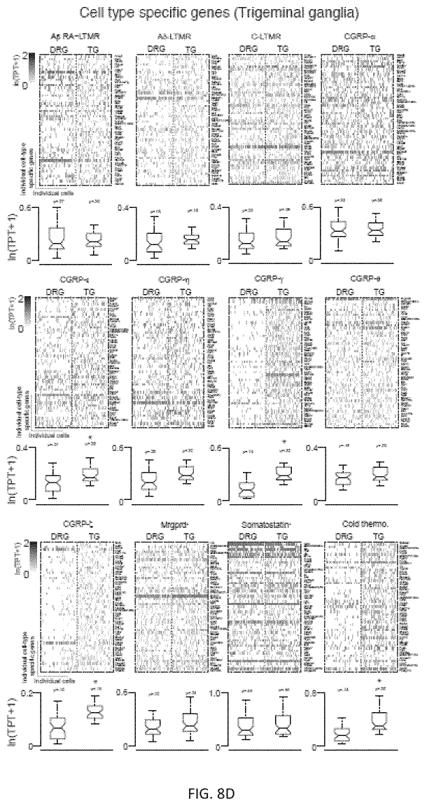

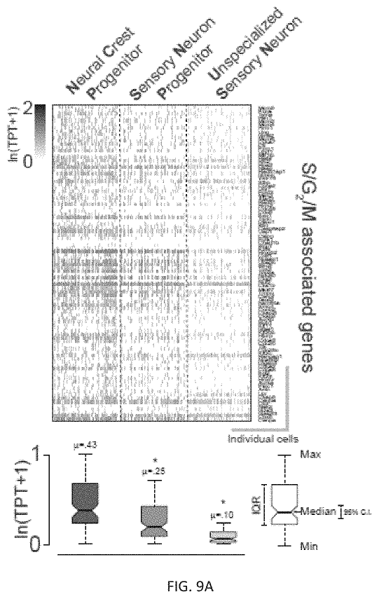

To begin to define transcriptional cascades underlying somatosensory neuron subtype specification, single-cell RNA sequencing (scRNA-seq) was performed at embryonic day 11.5 (E11.5), which is shortly after DRG formation, and at critical developmental milestones during somatosensory neuron development: at E12.5, when virtually all DRG neurons are post-mitotic⁹ and have extended axons well into the periphery; at E15.5, when peripheral and central target fields of somatosensory neurons are being innervated¹⁰¹¹; at P0, when maturation of sensory neuron endings within the skin and other targets is occurring^{12,13}; at P5, when peripheral endings have mostly refined into their mature morphological states and central projection terminals are properly organized within select spinal cord laminae^{8,14,15}; and in early adulthood (P28-42) (FIG. 1A, FIGS. 6A-6G). First, primary sensory neurons residing in young adult DRGs obtained from all axial levels were examined (FIG. 1A, FIG. 6A). Principal component analysis (PCA) and t-distributed stochastic neighbor embedding (t-SNE) were used to cluster adult DRG neurons based on the similarity of their transcriptomes (FIG. 1A). Each cluster was classified as a subtype based on prior studies that have described markers and functions for individual somatosensory neuron subtypes, in situ analysis confirmation, and by comparison to scRNA-seq generated from adult trigeminal ganglia (Methods, FIGS. 7A-7D, FIGS. 8A-8D, Table 1). These cell type classifications are consistent with previously published RNA-seq findings of adult DRG and trigeminal ganglia¹⁶⁻¹⁹.

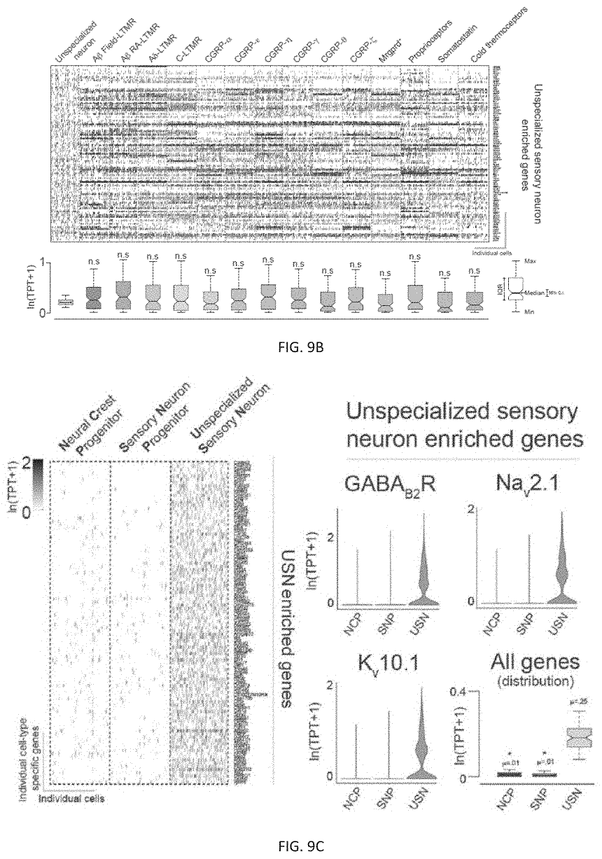

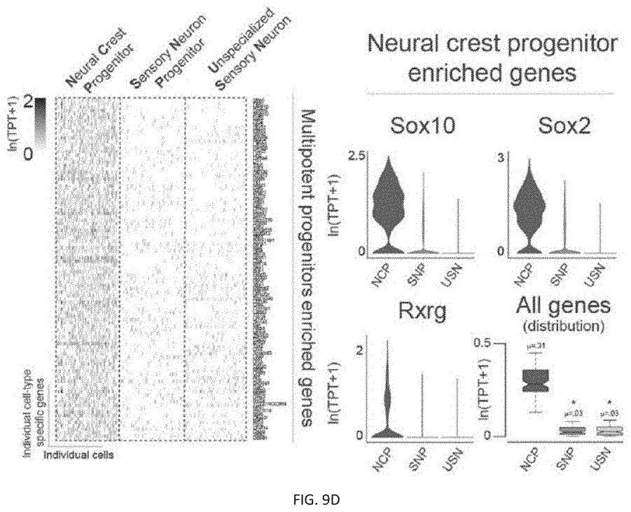

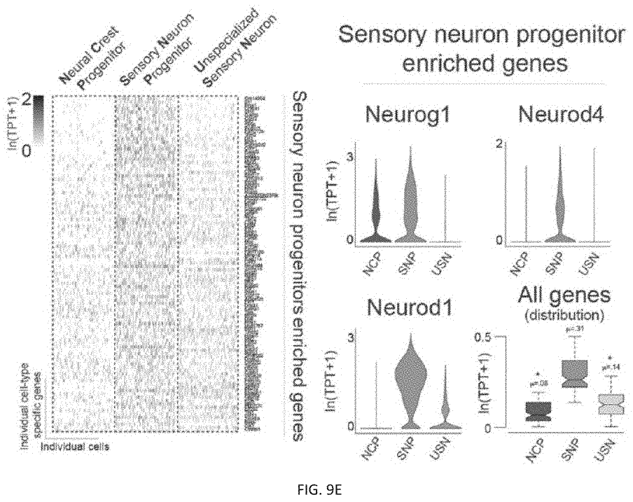

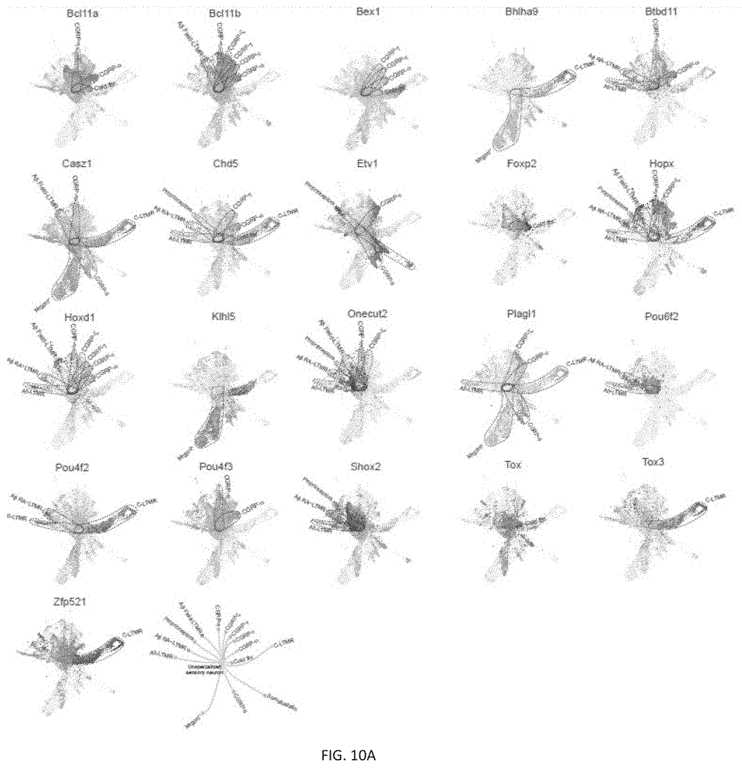

Next, how the transcriptional identities of mature somatosensory neuron subtypes compare to those of newborn sensory neurons by analyzing the transcriptomes of cells from DRGs at E11.5 (FIG. 1B) was sought. The E11.5 scRNA-seq data were visualized using uniform manifold approximation and projection (UMAP)²⁰. Mapping historically defined marker genes onto the UMAP representation revealed three principal cell types in E11.5 ganglia: 1) multipotent neural crest progenitors (NCPs), marked by Sox10²¹²²; 2) sensory neuron progenitors (SNPs), marked by Neurog1/2²³; and 3) nascent, postmitotic sensory neurons marked by expression of the somatosensory neuron gene Advillin (Avil²⁴) and concomitant loss of expression of cell-cycle associated genes (FIG. 1B and FIG. 9A). Monocle 3²⁵ was then used to infer developmental relationships between the NCPs, SNPs, and nascent Avil⁺ sensory neurons. This analysis revealed a single continuous trajectory connecting NCPs, SNPs, and Avil⁺ sensory neurons, suggesting a lack of transcriptional diversity in sensory neuron progenitors populating the Avil⁺ compartment. Surprisingly, descendants of Neurog1⁺ SNPs, labeled using Neurog1^{Cre}; Rosa26^{LSL-tdTomato} mice, were found to include a broad range of cell diameters (FIG. 1C), consistent with in vitro directed differentiation with Neurog1 overexpression leading to the generation of both small and large diameter DRG sensory neuron subtypes²⁶. Moreover, genes that are highly enriched in E11.5 Avil⁺ sensory neurons, relative to progenitors, generally remain expressed in adult somatosensory neuron subtypes (FIG. 9B). On the other hand, the majority of genes with expression patterns restricted to individual terminal somatosensory subtypes of adult ganglia were expressed at trace levels in E11.5 Avil⁺ sensory neurons (FIG. 1D), suggesting that upon cell-cycle exit sensory neurons are transcriptionally unspecialized specifically with respect to subtype specific genes. These observations led to considering whether a ‘transcriptionally unspecialized state’ serves as the starting point for somatosensory subtype diversification.

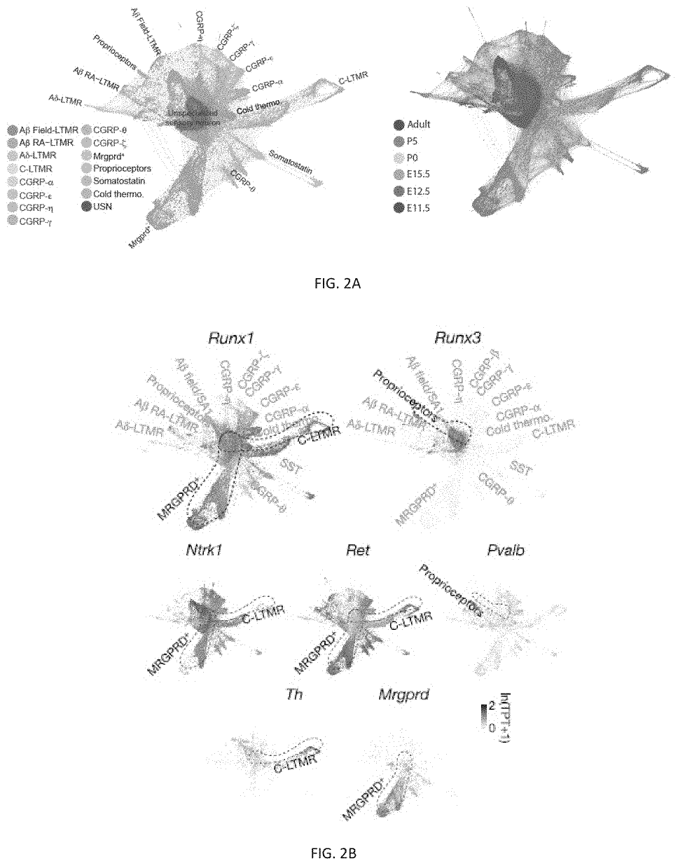

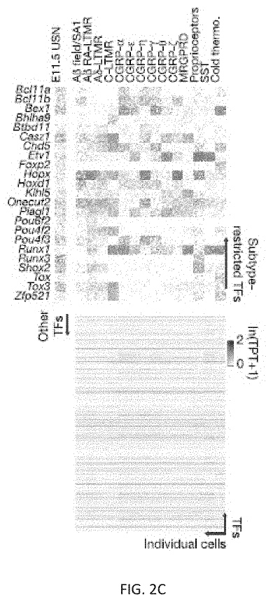

To address this, scRNA-seq transcriptomes generated from sensory neurons between E11.5 and adulthood were compared. Prospective identities for sensory neurons at each developmental stage were assigned based on transcriptional similarity using canonical correlation analysis²⁷ (FIG. 1A and FIG. 6F) as well as a graph-based strategy for locally embedding consecutive timepoints based on the transcriptional variation they share. Single-cell k-nearest neighbor graphs were constructed for each timepoint (tᵢ) with nodes representing cells and edges linking neighbors. These graphs were then joined by identifying neighboring cells in adjacent timepoints using a coordinate system learned from the subsequent timepoint (tᵢ₊₁) (Methods). The resulting graph forms a branching network that can be visualized using a force-directed layout. This representation spans all developmental stages and provides a consolidated view of the transcriptional maturation of each principal somatosensory neuron subtype from E11.5 to adulthood (FIG. 2A).

Next, whether this graph-based representation of developmental gene expression profiles of sensory neuron subtypes recapitulates known developmental relationships was tested. The expression patterns of the TFs Runx1 and Runx3 were inspected, which are implicated in development of select unmyelinated (C-fiber) neuron subtypes and proprioceptors, respectively^{28-30.} It was found that Runx1 expression was prominent in unmyelinated sensory neuron subtypes, whereas Runx3 expression was restricted to mature proprioceptors of adult ganglia, as previously described^{28,29} (FIG. 2B). Furthermore, the graph-based representation accurately depicts the developmental switch from Ntrk1⁺ to Ret⁺ known to occur in subsets of non-peptidergic C-fiber neurons³⁰ (FIG. 2B). To facilitate exploration of this dataset by the community, an HTML was created based interactive interface enabling visualization of the expression pattern of any gene at each developmental time point, from E11.5 to adulthood, for each of the somatosensory neuron subtypes.

### TFs in Sensory Neuron Development

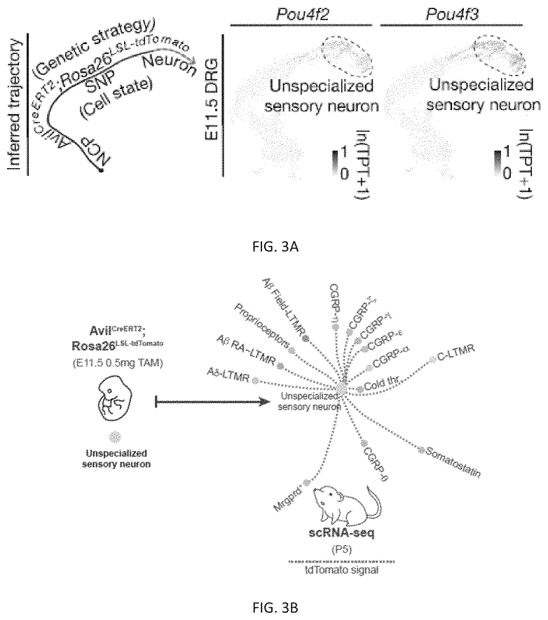

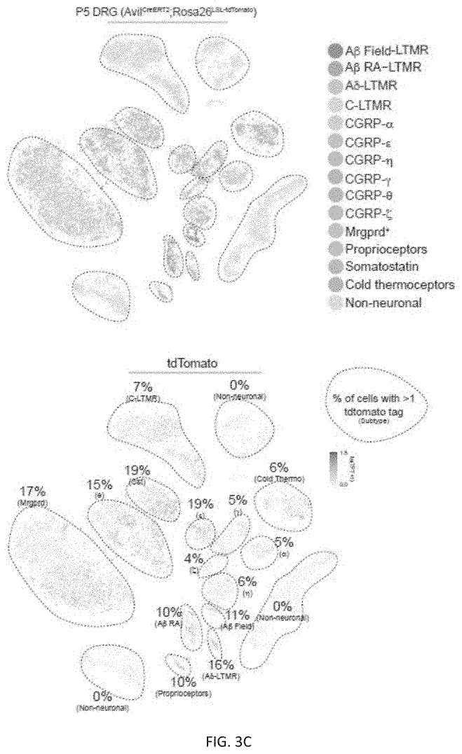

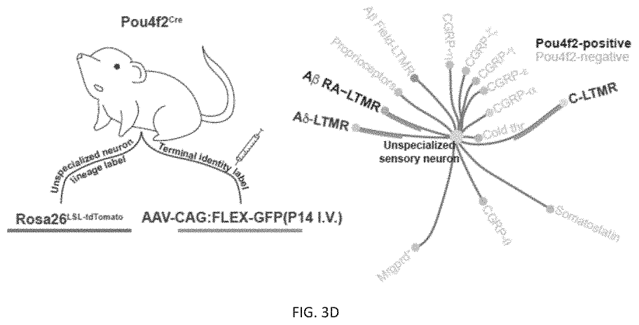

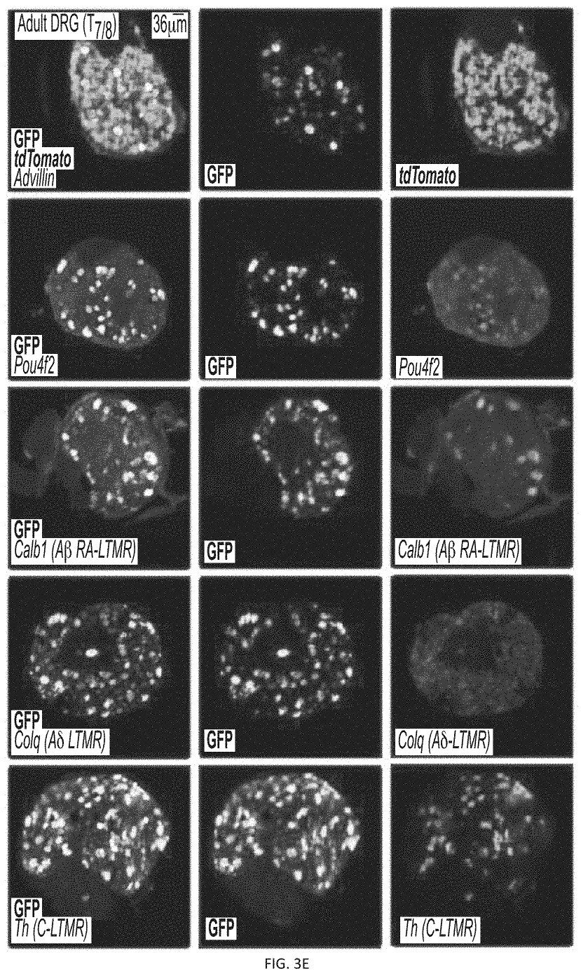

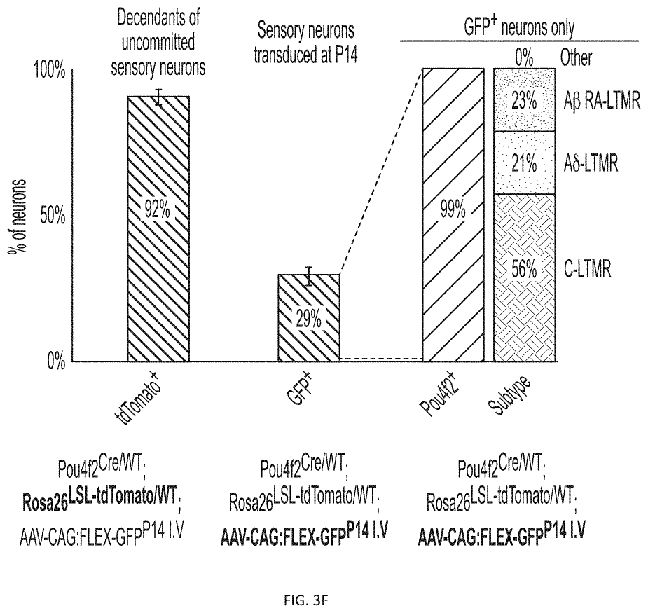

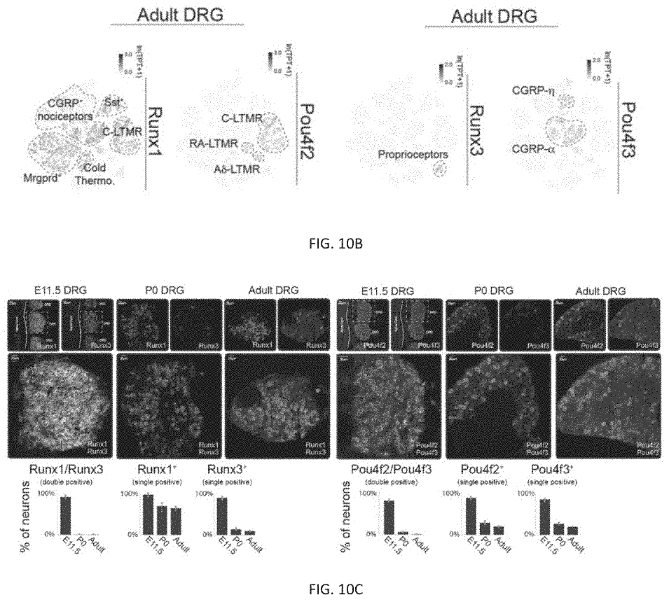

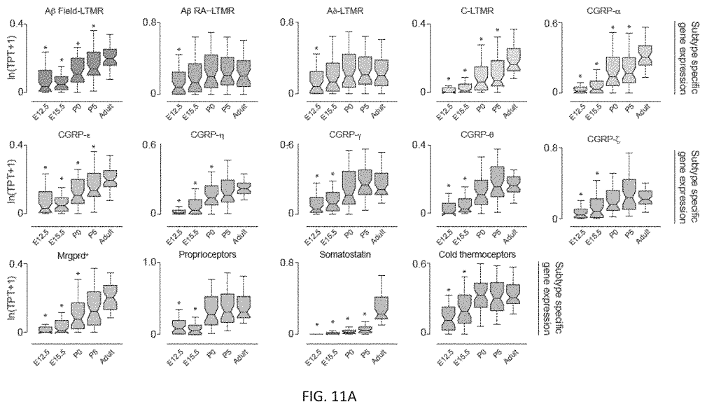

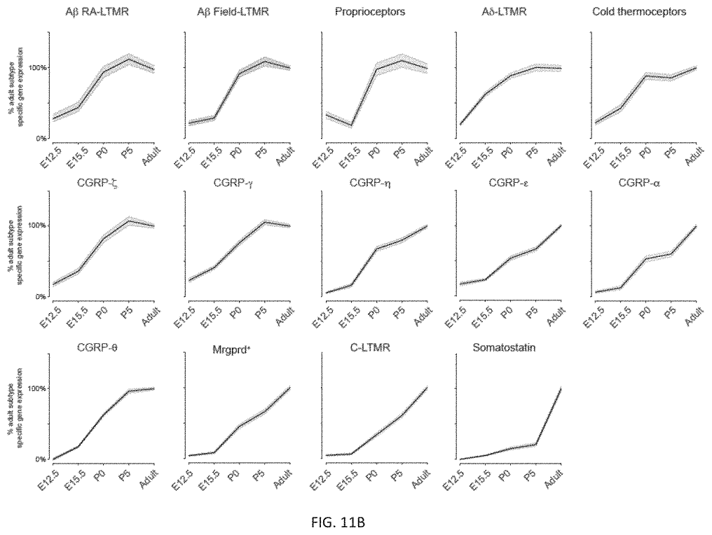

One observation from the initial analysis of the graph-based representation of developmental transcriptomes of sensory neurons is that TFs implicated in development of sensory neuron subtypes, Runx1 and Runx3, are broadly co-expressed in nascent E11.5 Avil⁺ sensory neurons, which stands in contrast to their mutually exclusive expression patterns in terminally differentiated subtypes of adult DRGs (FIG. 2B). This is consistent with the finding that Runx1 and Runx3 proteins are co-localized in embryonic DRG³¹. This observation led to the consideration of whether other TFs that are subtype-restricted in adult ganglia may be co-expressed in nascent, transcriptionally unspecialized sensory neurons. To address this possibility, TFs were identified beyond Runx1 and Runx3 that are expressed in select somatosensory neuron subtypes of mature ganglia by inspecting 1152 neuronally expressed TFs and found that 23 are expressed in distinct subsets of adult somatosensory neurons (FIG. 2C). Strikingly, as observed with Runx1 and Runx3, the scRNA-seq data revealed that several TFs expressed in select subtypes of sensory neurons of mature DRGs are co-expressed in newborn E11.5 sensory neurons (FIG. 10A). These scRNA-seq findings were verified using double single-molecule RNA fluorescent in situ hybridization (smRNA-FISH), with Runx1 and Runx3 as well as Pou4f2 and Pou4f3 serving as test cases. Indeed, smRNA-FISH measurements showed that Runx1 and Runx3 as well as Pou4f2 (Aβ RA-LTMRs, Aδ-LTMRs, C-LTMRs) and Pou4f3 (CGRP-αs and CGRP-ηs) are co-expressed in the majority of E11.5 Avil⁺ sensory neurons, despite their mutually exclusive expression patterns in neurons of P0 and adult ganglia (FIGS. 10B-10C). These observations suggest that all somatosensory neuron subtypes transit through a postmitotic transcriptionally unspecialized state. To further address this, descendants in the Avil⁺ cell compartment at E11.5 were genetically labelled by administering a low dose of tamoxifen (0.5 mg) at E11.5 to Avil^{CreERT2}; Rosa26^{LSL-tdTomato} mice and found tdTomato transcripts to be present in 5-19% of cells in each somatosensory neuron subtypes of adult ganglia by scRNA-seq (FIGS. 3A-3C). In addition, all descendants of one of the ‘broad early’-to-‘subtype restricted late’ TFs, Pou4f2, were labelled with tdTomato using a Pou4f2^{Cre}; Rosa26^{LSL-tdTomato} mouse line, and terminally differentiated Pou4f2⁺ DRG subtypes were transduced in the same mouse using an AAV carrying a Cre-dependent GFP reporter delivered at P14 (FIG. 3D). smRNA-FISH analysis revealed tdTomato transcripts in >90% of DRG sensory neurons in adult Pou4f2^{Cre}; Rosa26^{LSL-tdTomato}; AAV-CAG:FLEX-GFP^{P14 LV} mice while, in contrast, GFP transcripts were restricted to Aβ RA-LTMRs, Aδ-LTMRs and C-LTMRs (mature Pou4f2⁺ populations) (FIG. 3E). Interestingly, a developmental analysis of subtype-specific gene expression revealed that, in general, large diameter neurons achieve transcriptional maturity prior to small diameter neurons, consistent with the historical view^{32,33} (FIGS. 11A-11B). Together, these experiments indicate that cells in the transcriptionally unspecialized compartment express a broad array of TFs that become restricted to select subsets of sensory neurons as development proceeds.

### Specification of Subtype Identity

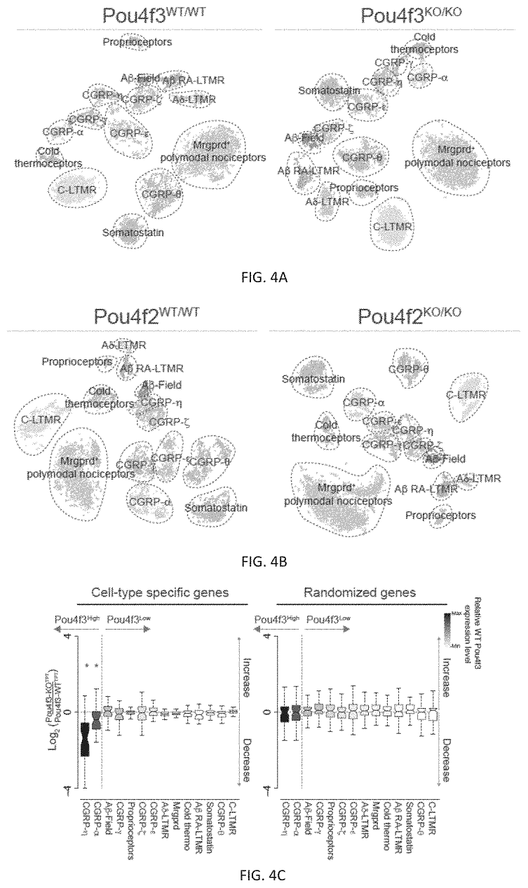

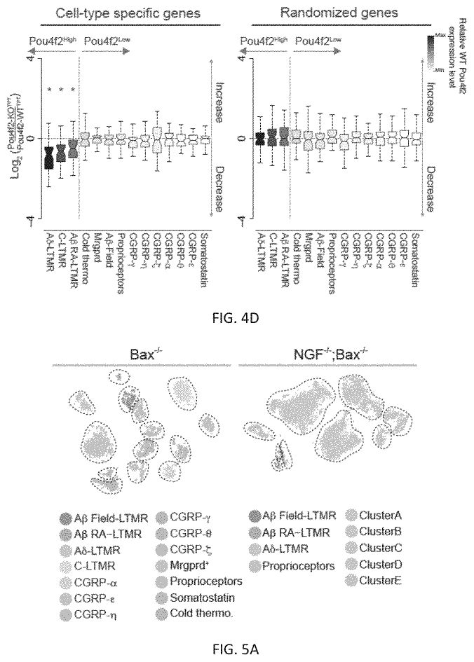

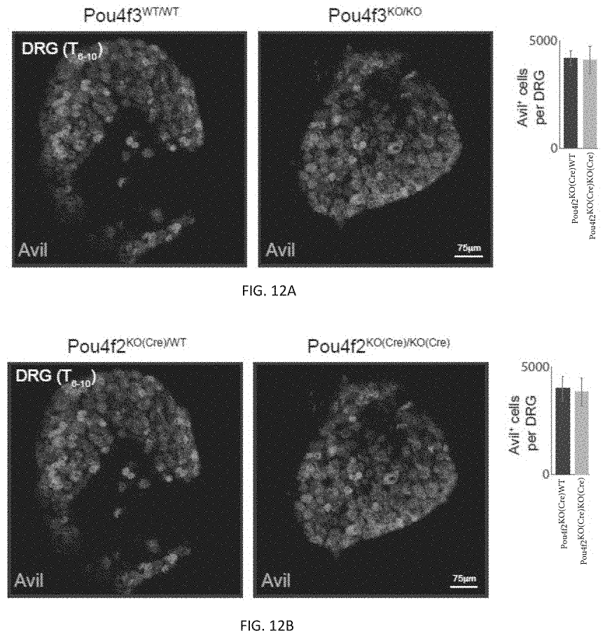

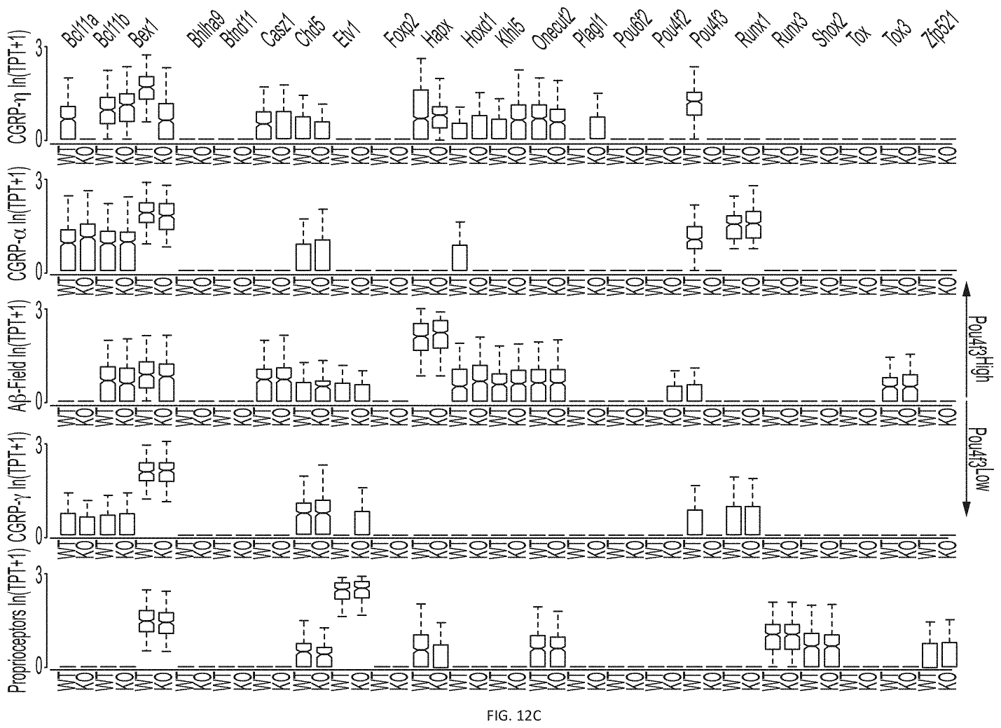

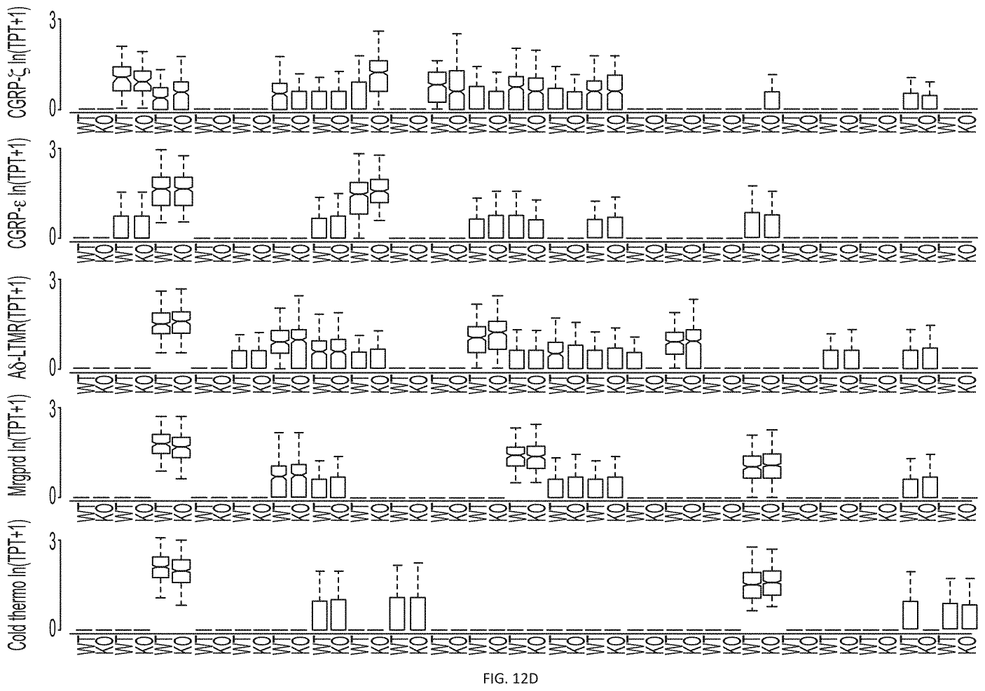

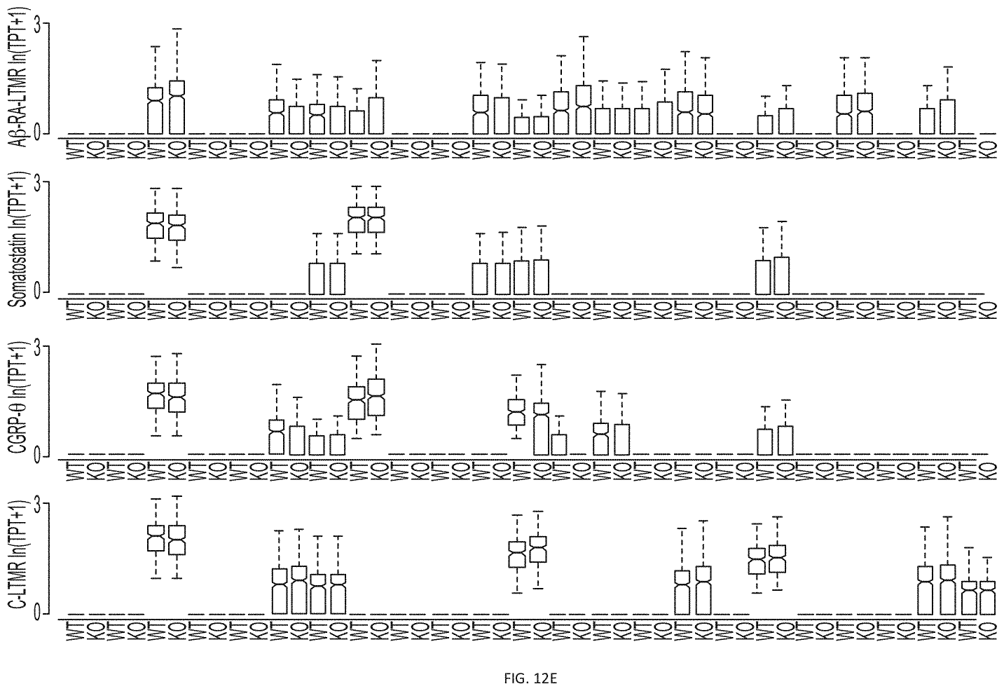

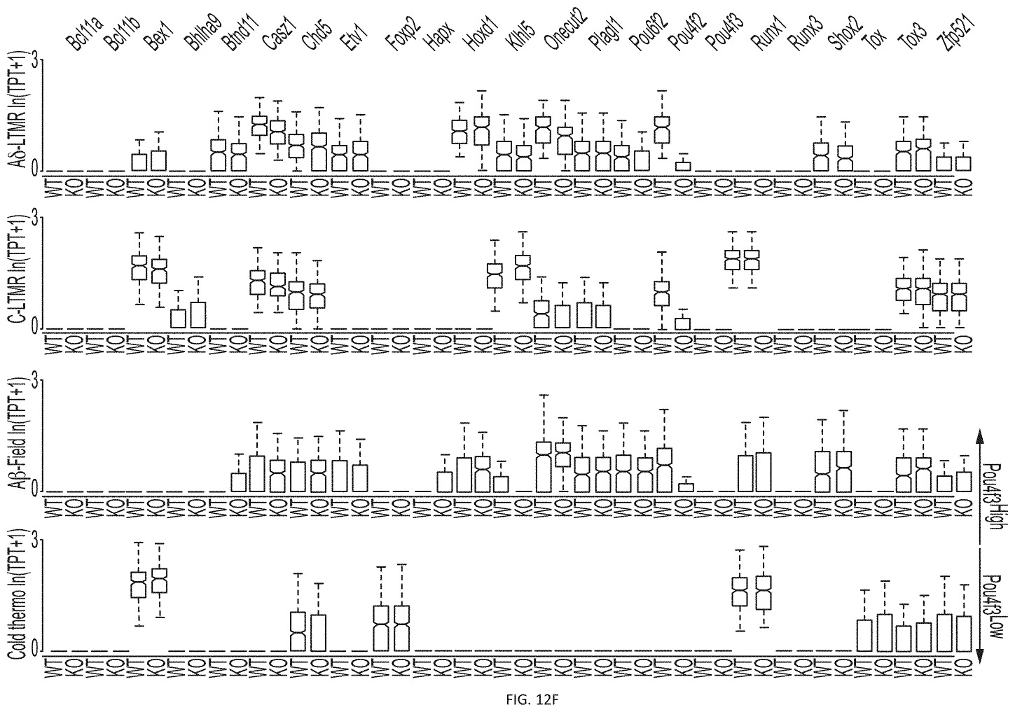

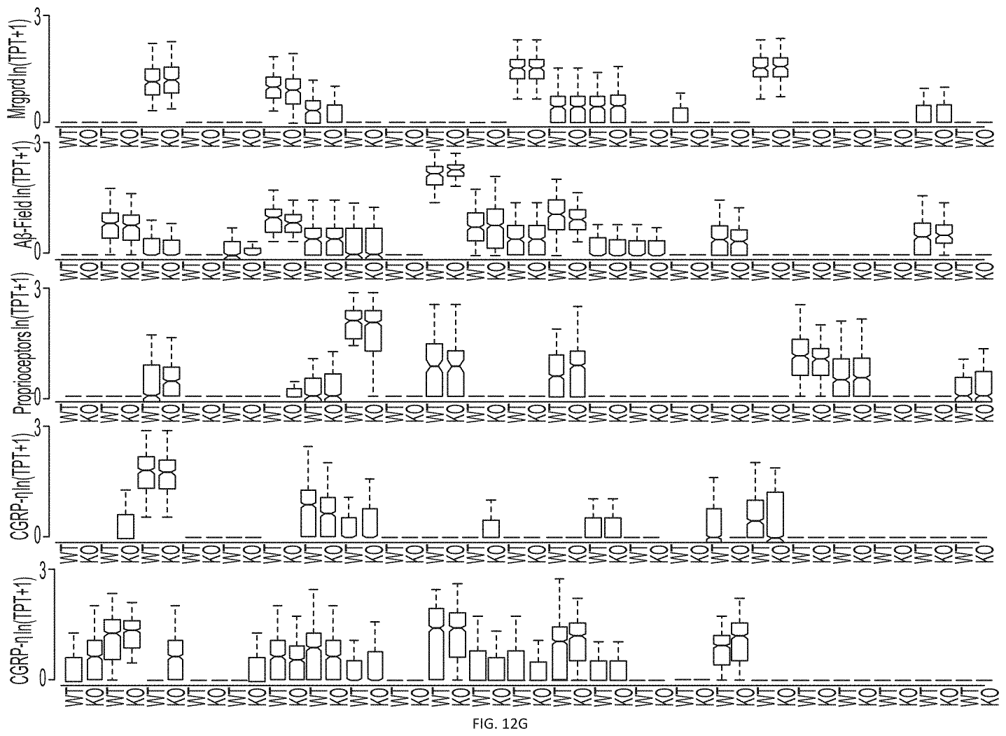

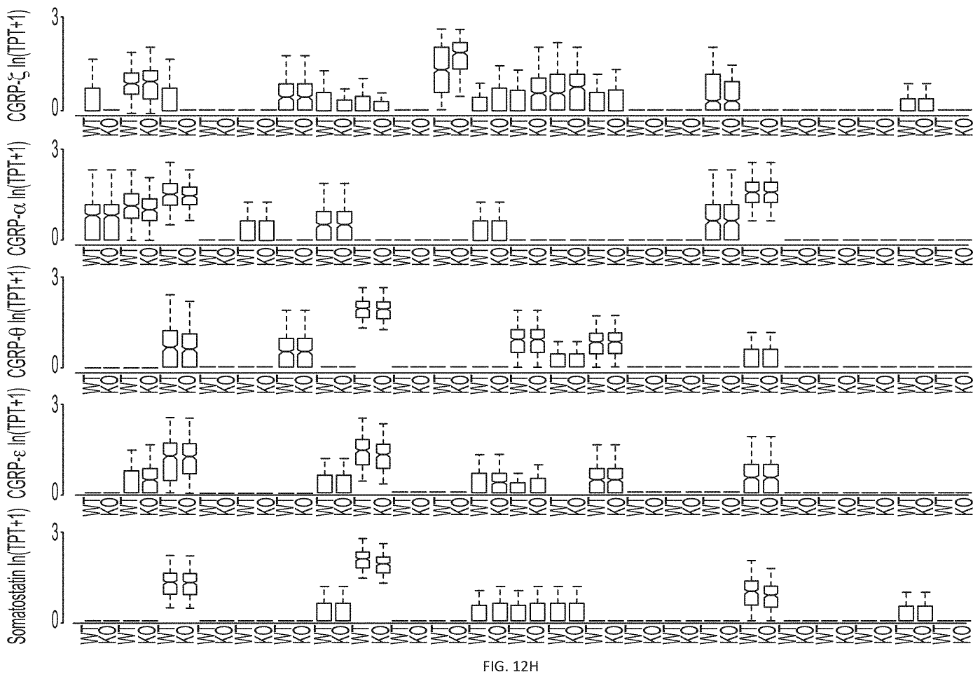

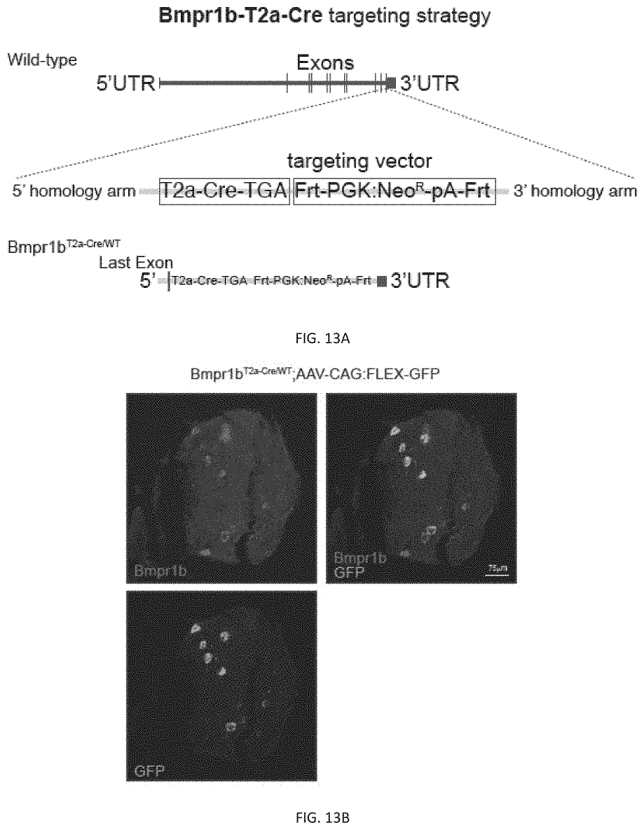

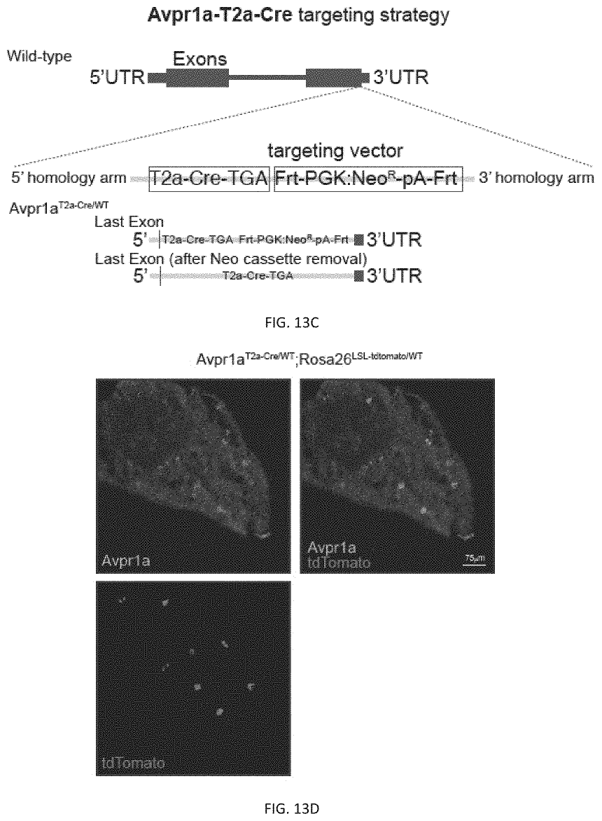

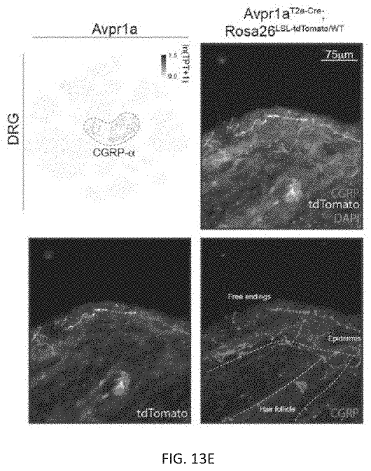

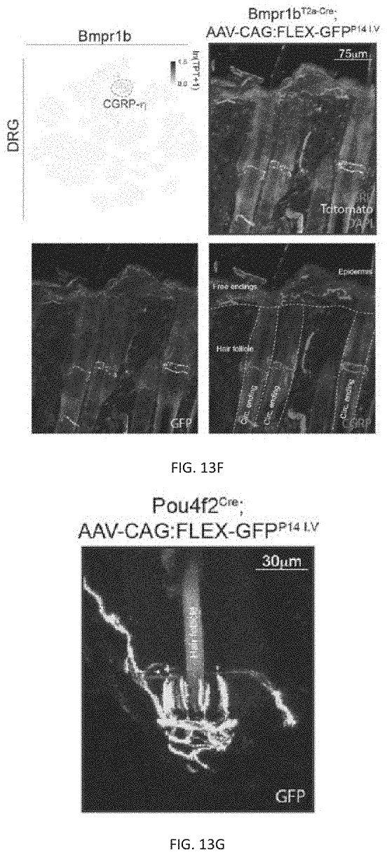

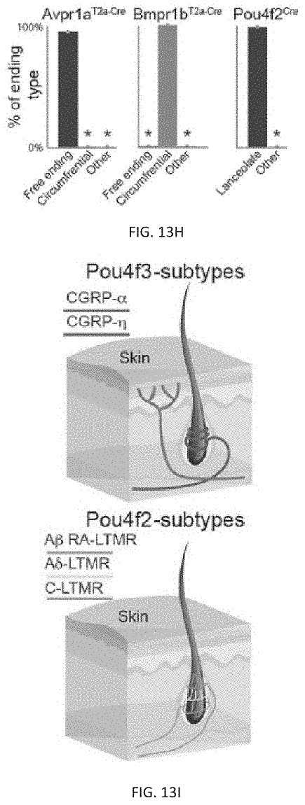

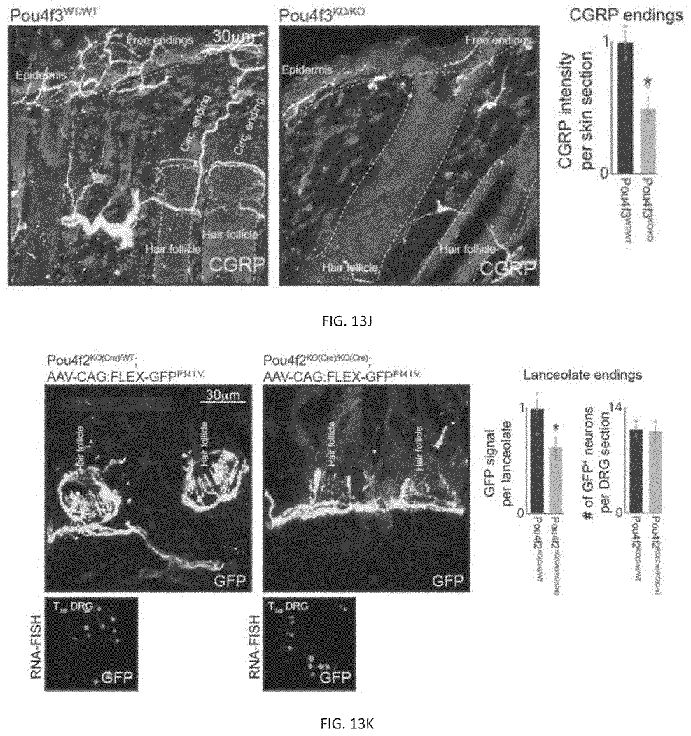

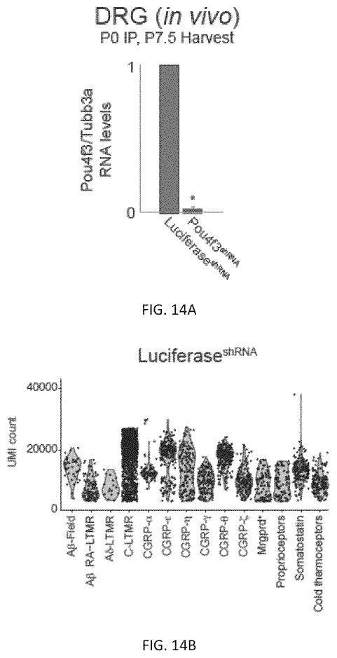

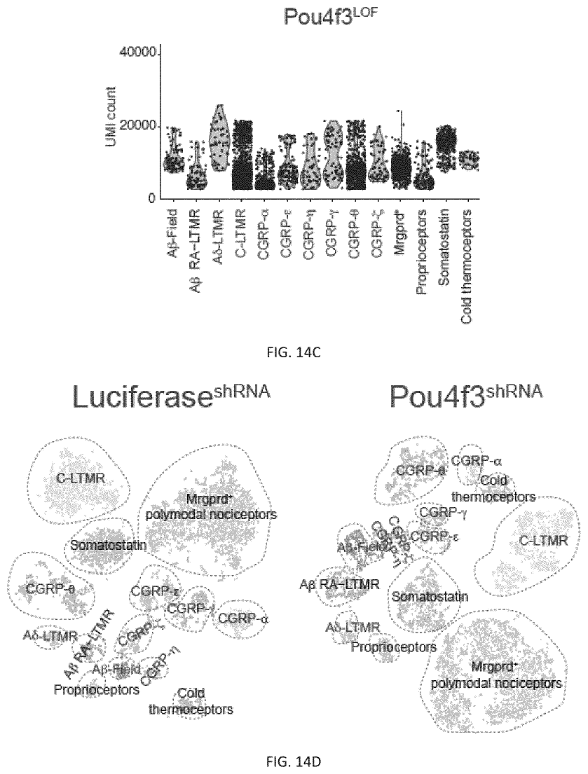

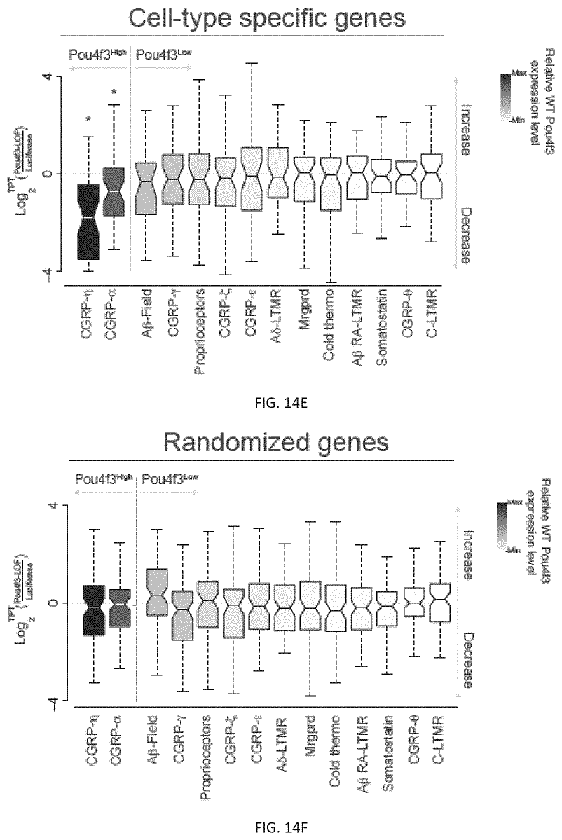

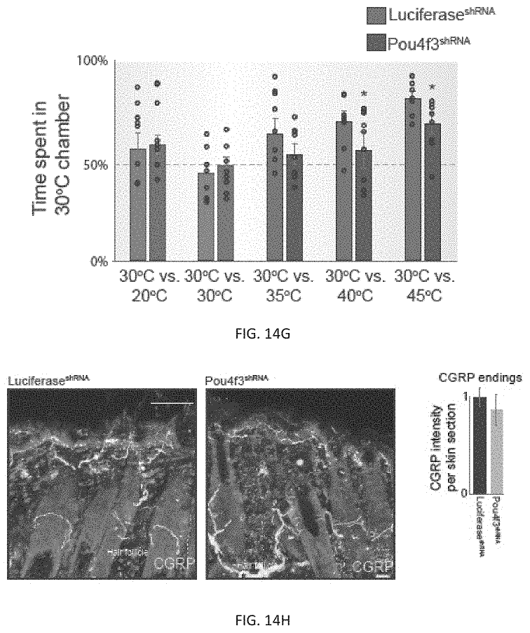

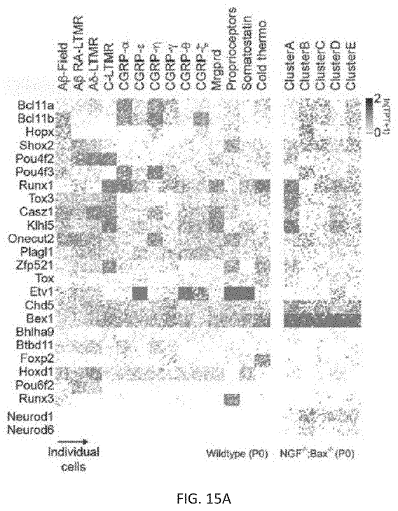

Next, it was asked if broad-to-restricted TFs contribute to sensory neuron diversification during the transcriptionally unspecialized state, thus broadly influencing transcriptional maturation of sensory neurons, or whether these TFs primarily influence the subtypes in which their expression is maintained. DRGs were harvested from neonatal (P0-5) pups harboring null alleles of either Pou4f2 or Pou4f3, which are representative broad-to-restricted TFs, and generated scRNA-seq transcriptomes from Pou4f2^{KO(Cre)/KO(Cre)} mice and Pou4f2^{++/}littermate controls as well Pou4f3^{-/-} mice and Pou4f3^{++/}littermate controls. Initial inspection of the scRNA-seq data obtained from both Pou4f2 and Pou4f3 mutant animals revealed clusters corresponding to each somatosensory subtype (FIG. 4A-4B). It was found that cell numbers were not compromised as representative ganglia (T_{7/8}) from Pou4f2 or Pou4f3 knockouts have similar numbers of neurons compared to littermate controls (FIGS. 12A-12B). Importantly, subtype-specific genes in both the Pou4f2⁺ populations and Pou4f3⁺ populations were reduced in the respective knockouts, compared to littermate controls (FIGS. 4C-4D), whereas randomly selected genes were unchanged (FIGS. 4C-4D). In contrast, somatosensory neuron subtypes that normally extinguish expression of Pou4f2 and Pou4f3 after E11.5 generally exhibited less dramatic alterations to subtype-specific gene expression or subtype-restricted TF expression (FIGS. 4C-4D, FIGS. 12C-12H). Given the reduction of subtype-specific gene expression in Pou4f2 and Pou4f3 mutants, the consequences of Pou4f2 or Pou4f3 ablation on the unique axonal endings associated with mature somatosensory neuron subtypes were also determined. Although the axonal endings associated with Pou4f2⁺ subtypes are known to form longitudinal lanceolate endings around hair follicles¹, the axonal ending morphologies associated with the Pou4f3⁺ subtypes were not known. Genetic labeling experiments using newly generated Cre lines for each Pou4f3⁺ subtype revealed that the axonal ending types of CGRP-α neurons are free nerve endings that penetrate the epidermis whereas CGRP-η neurons form circumferential endings associated with hair follicles (FIGS. 13A-13D). It was found that the longitudinal lanceolate endings and CGRP circumferential axonal endings were partially compromised in Pou4f2 and Pou4f3 knockout mice, respectively (FIGS. 13E-13K). Furthermore, postnatal depletion of Pou4f3 with shRNA altered subtype-specific gene expression and function (FIGS. 14A-14H). Taken together, two representative subtype-restricted TFs, Pou4f2 and Pou4f3 control transcriptional maturation of the sensory neuron subtypes in which they remain expressed.

### Extrinsic Control of Subtype Identity

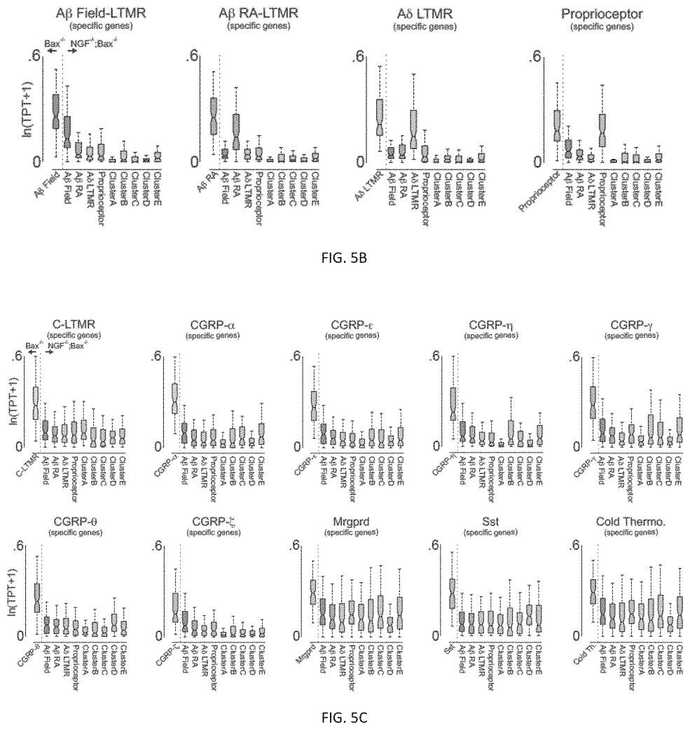

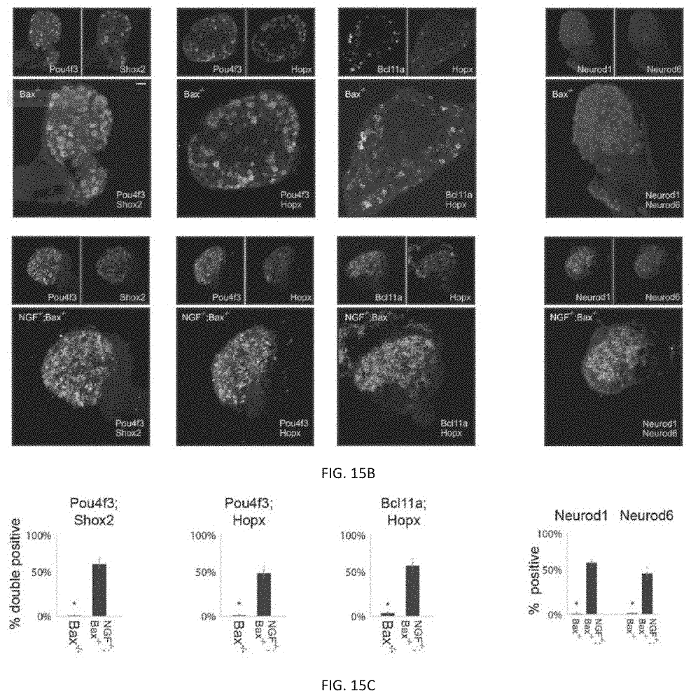

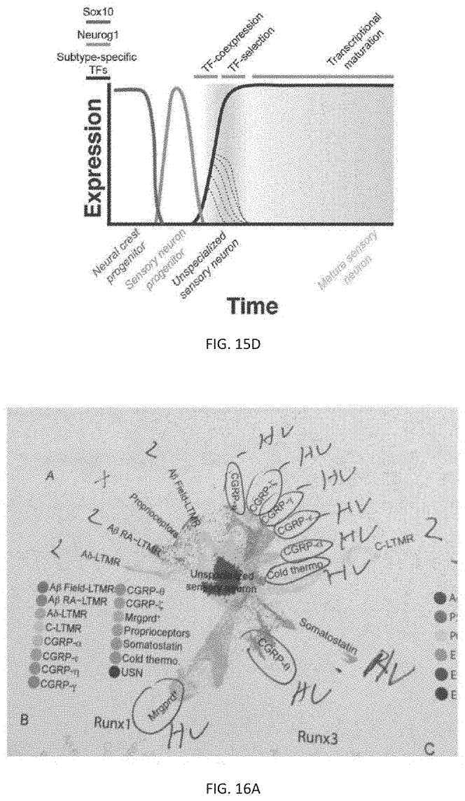

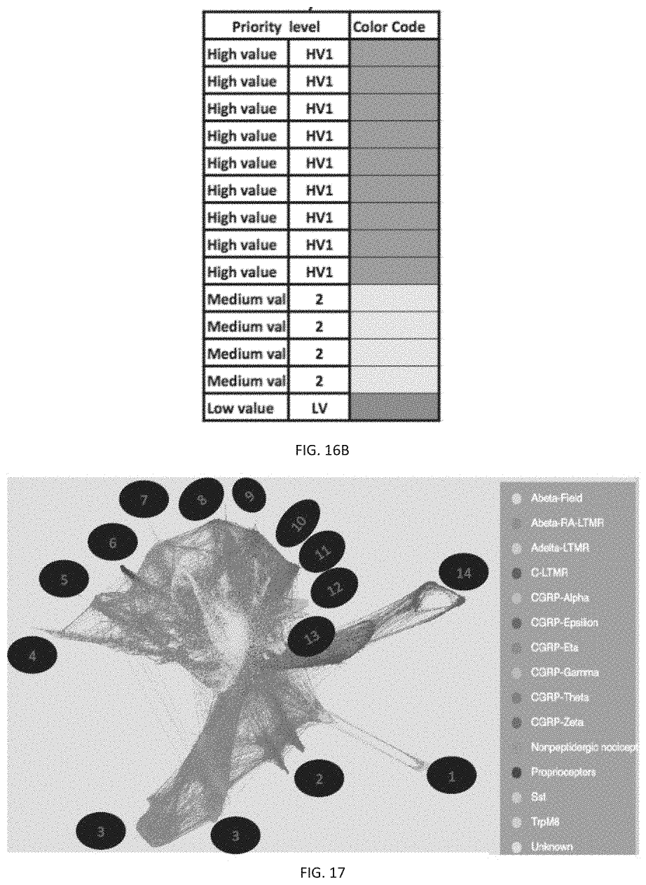

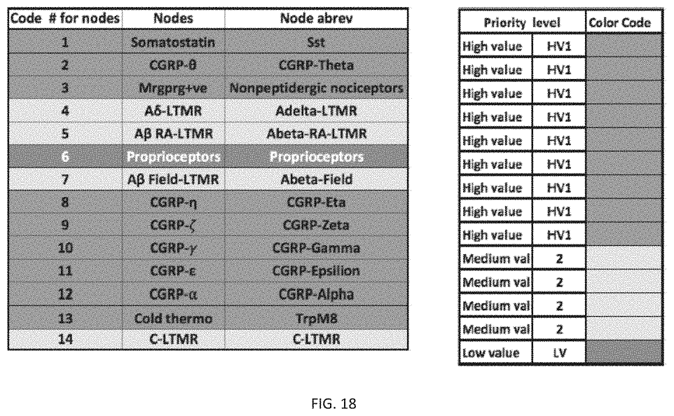

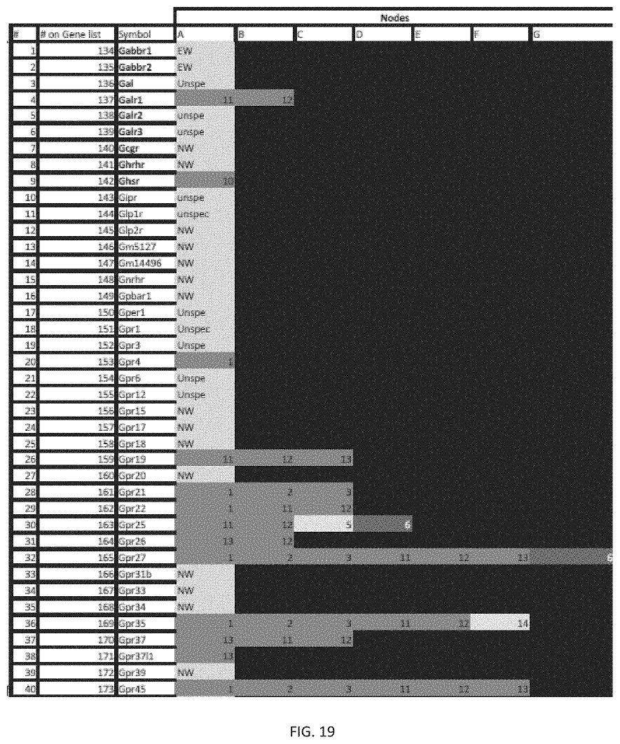

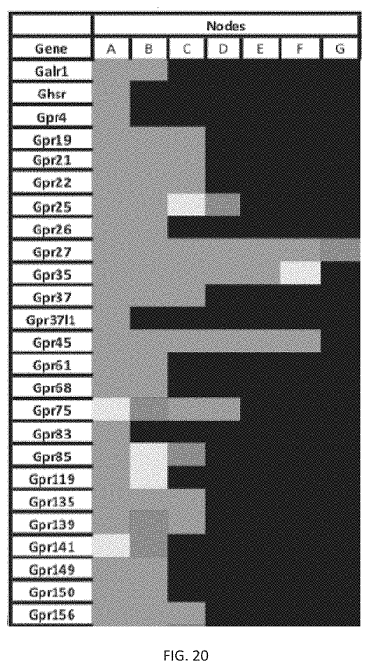

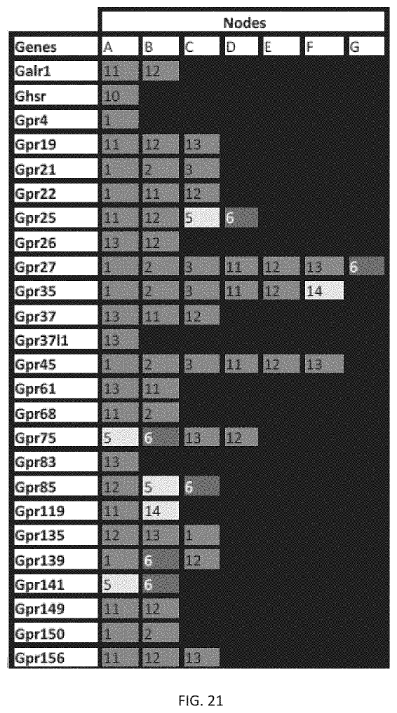

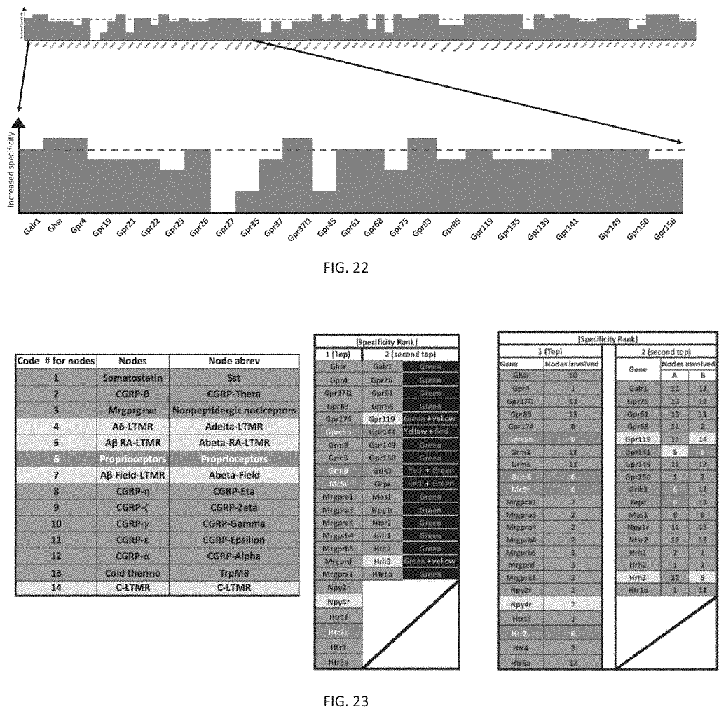

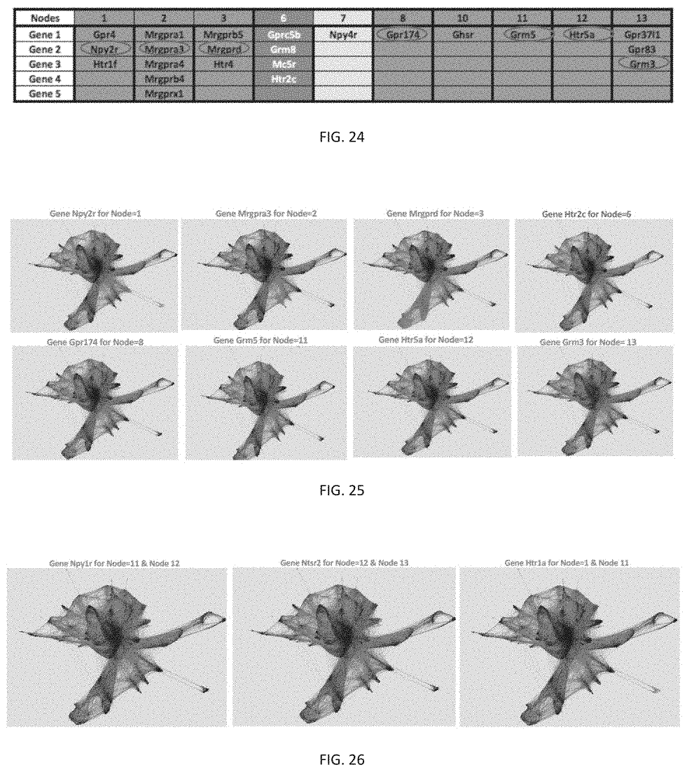

Whether differential maintenance or extinction of TFs in emerging subtypes occurs via a process that is entirely intrinsic to developing sensory neurons or guided by extrinsic cues was next addressed. The mesenchymal and epidermal environments through which embryonic somatosensory axons extend are rich sources of extrinsic signals including neuronal growth factors⁸. Therefore, whether nerve growth factor (NGF), an extrinsic cue critical for growth and survival of Ntrk1 (TrkA; NGF-receptor)-expressing embryonic somatosensory neurons³⁴, which represent ˜80% of the adult DRG, may exert control over the TF selection process, was sought. To address this, scRNA-seq was performed using DRGs from neonatal mice harboring a targeted mutation in the NGF gene. This genome-wide analysis of NGF-dependent gene expression was done using the apoptosis deficient Bax-knockout genetic background to circumvent the apoptotic cell death of DRG neurons associated with developmental loss of NGF³⁵. While clustering analysis of the scRNA-seq data revealed that all somatosensory neuron subtypes are present in Bax^{-/-} controls (FIG. 5A), fewer transcriptionally distinct neuronal populations were observed in NGF^{-/-}; Bax^{-/-} double mutants (FIG. 5A). Ntrk1-negative populations (proprioceptors and A-fiber mechanoreceptors) were not dramatically transcriptionally compromised in NGF^{-/-}; Bax^{-/-} mutants compared to Bax^{-/-} controls (FIG. 5B), as expected. However, subtype-specific gene expression patterns normally present in Ntrk1⁺ sensory neuron subtypes were dramatically altered in the NGF^{-/-}; Bax^{-/-} mutants (FIG. 5C). Importantly, examination of the aforementioned subtype-restricted TFs showed that the combinations of TFs expressed in the unidentified neuronal clusters in NGF^{-/-}; Bax^{-/-} mutants bore no resemblance to the TF combinations observed in neuronal subtypes of control animals, which was confirmed using smRNA-FISH analyses (FIG. 15A). Furthermore, members of the Neurod family of transcription factors, which are normally extinguished during embryonic development, remained expressed at P0 in NGF^{-/-}; Bax^{-/-} mutants (FIGS. 15B-15D). These findings indicate that the selection of somatosensory neuron subtype-restricted TFs is controlled, at least in part, by extrinsic cues acting on nascent sensory neurons.

### DISCUSSION

The genome-wide transcriptomic analyses of cells traversing somatosensory neuron developmental stages support a model in which newborn somatosensory neurons are unspecialized with respect to expression of subtype-restricted TFs, and that differential maintenance of unique combinations of these subtype-restricted TFs enables nascent sensory neurons to resolve into mature subtypes (FIG. 15D). Early co-expression, and subsequent resolution, of TFs has been proposed to underlie diversification of stem cells in the hematopoietic lineage³⁶⁻³⁸, neural crest progenitors prior to lineage committment³⁹, and developing spinal motor neurone⁴⁰⁻⁴², although this view has been challenged in the case of the hematopoietic system⁴³. The diversification of somatosensory neurons shares commonalities and differences with these systems. Unlike cells of the early neural crest and the hematopoietic lineages, it was proposed that somatosensory neuron subtypes emerge following cell cycle exit, and unlike other progenitor types, newborn, post-mitotic Avil⁺ somatosensory neurons are not migratory but rather permanent residents of sensory ganglia. Therefore, nascent sensory neurons cannot rely on cell division or migration to encounter new environments. Rather, a feature of nascent somatosensory neurons is that they immediately extend axons along intermediate targets, such as large blood vessels, en route to target organs, such as the skin, where they encounter extrinsic cues, including NGF and other secreted factors. A model was proposed in which multiple distinct extrinsic cues act on axons of transcriptionally unspecialized sensory neurons, depending on the timing and trajectories of their projection patterns. These cues function, in part, to resolve TF expression patterns from a co-expressed state to a subtype-restricted state to promote the transcriptional specializations underlying the unique molecular, morphological, and physiological properties of somatosensory neuron subtypes.

### Methods

Animals

All mouse experiments in this study were approved by the National Institutes of Health and the Harvard Medical School IACUC. Experiments followed the ethical guidelines outlined in the NIH ‘Guide for the care and use of laboratory animals (grants.nih/gov/grants/olaw/guide-for-the-care-and-use-of-laboratory-animals.pdf). Avpr1a and Bmpr1b^{T2a-Cre} mice were generated using standard homologous recombination techniques in ES cells. Chimeras were generated by blastocyst injection and subsequent germline transmission was confirmed by tail PCR. The neo selection cassette was excised using a Flp-deleter strain for the Avpr1a^{T2a-Cre} but left intact for the Bmpr1b^{T2a-Cre} lines. Mice were housed under standard conditions and provided chow and water ad libitum. Plug date was considered embryonic day 0.5 (E0.5) and date of birth was considered postnatal day 0 (P0). Pou4f3 null mice were obtained from Jax (Stock No. 008645). Pou4f2 null(Cre) mice were obtained from Jax (Stock No. 030357). Rosa26 Cre-dependent tdtomato reporter mice were obtained from Jax (Stock No. 007914). AvilCreERT2 mice were obtained from Jax (Stock No. 032027). All experiments with wild-type animals were conducted with mice on the C57Bl/6J background and were obtained from Jackson Laboratory.

Dissociation and Purification of Isolated Single Sensory Neurons.

The dissection strategy used were nearly identical for all ages presented in this study. Specifically, animals were sacrificed, and spinal columns were removed and placed on a tray of ice. Individual DRGs with central and peripheral nerves attached were removed from all axial levels and placed into ice-cold DMEM:F12 (1:1) supplemented with 1% pen/strep and 12.5 mM D-Glucose. A fine dissection was performed to remove the peripheral and central nerve roots, resulting in only the sensory ganglia remaining. 200-400 individual ganglia were collected for the DRG and 20-30 ganglia for the trigeminal for each bioreplicate of single-cell sequencing. All scRNA-seq experiments in this study were performed with >2 bioreplicates. Sensory ganglia were dissociated in 40 units papain, 4 mg/ml Collagenase, 10 mg/mL BSA, 1 mg/mL hyalurdonidase, 0.6 mg/mL DNAse in DMEM:F12+1% pen/strep+12.5 mM glucose for 10 minutes at 37° C. Digestion was quenched using 20 mg/mL ovomucoid (trypsin inhibitor), 20 mg/mL BSA in DMEM:F12+1% pen/strep+12.5 mM glucose. Ganglia were gently triturated with fire polished glass pipettes (opening diameter of approx. 150-200 μm). Neurons were then passed through a 70μm filter to remove cell doublets and debris. Neurons were pelleted and washed 4-8× in 20 mg/mL ovomucoid (trypsin inhibitor), 20 mg/mL BSA in DMEM:F12+1% pen/strep+12.5 mM glucose followed by 2× washes with DMEM:F12+1% pen/strep+12.5 mM Glucose all at 4 C. After washing, cells were resuspended in 50-200 uL of DMEM:F12+1% pen/strep+12.5 mM glucose. Cells were counter stained with Trypan blue, visually inspected, counted with a hemocytometer. Dissociated ganglia preparations were considered to pass quality control and used for scRNA-seq if >90% of cells were viable, as measured by exclusion of trypan blue and virtually no cellular debris was visible.

Tissue Processing for RNA Florescent In Situ Hybridization (RNA-FISH).

For sample preparation, individual DRGs from mice were rapidly dissected and axial level was identified by identifying specific DRGs using the T12 DRG as a landmark. The T12 DRG was defined as the ganglia immediately caudal to the last rib. DRGs were frozen in dry-ice cooled 2-metylbutane and stored at −80° C. until sectioned. DRGs were sectioned at a thickness of 15-20 μm and RNAs were detected by RNAscope (Advanced Cell Diagnostics) using the manufacturer's protocol. Total numbers of neurons per section of DRG were estimated by counting neuronal nuclei as measured by DAPI and counts were confirmed as reasonable estimates by comparing to counts measured by measuring Advillin or Pou4f/Brn3a, which are both pan-somatosensory neuron markers. It was observed that somatosensory neuron number per section were similar for DAPI vs Advillin or Pou4f1/Brn3a. The following probes were used: Mm-Th (Cat #: 317621), Mm-Calb1 (Cat #-428431), Mm-Pou4f2 (Custom made), Mm-Pou4f3 (Custom made), Mm-Avil (Cat #: 498531), Mm-Asic1 (Cat #: 480581), Mm-Mrgpra3 (Cat #: 548161), Mm-Pou4f1 (Cat #: 414671), Mm-Colq (Cat #: 496211), Mm-Sst (Cat #: 404631), Mm-Pvalb (Cat #: 421931), Mm-Ikzf1 (Cat #: 511201), Mm-Avpr1a (Cat #: 418061), Mm-Oprk1 (Cat #: 316111), Mm-Mrgprd (Cat #: 417921), Mm-Bmpr1b (Custom made), Mm-Vcan (Cat #: 486231), Mm-Trpm8 (Cat #: 420451), Mm-Neurod1 (Cat #: 416871), Mm-Neurod6 (Cat #: 444851), Mm-Shox2 (Cat #: 554291), Mm-Hopx (Cat #: 405161), Mm-Runx1 (Cat #: 406671), Mm-Runx3 (Cat #: 451271) GFP (Cat #: 400281), tdTomato (Cat #: 317041).

Single-Cell RNA Library Preparation, Sequencing, and Analysis.

Single cell RNA-seq was performed with the 10× Genomics Chromium Single Cell Kit (v2 & v3). Approximately 1000-8000 cells were added to the RT mix prior to loading on the microfluidic chip. Downstream reverse transcription, cDNA synthesis/amplification, and library preparation were performed according to manufacturer's instructions. All samples were sequenced on a NextSeq 500 with 58 bp sequenced into the 3′ end of the mRNAs. Initial gene expression tables for individual barcodes were generated using the cellranger pipeline according to instructions provided by 10× Genomics. All gene expression tables were then imported into R and analyzed with Seurat (v 2.3) with standard procedures. Cluster identification: clusters were classified into transcriptionally distinct somatosensory neuron subtypes: Aβ RA-LTMRs⁴⁴⁻⁴⁶, Aβ Field-LTMRs/Aβ SA1-LTMRs^{46,47}, Aδ-LTMRs^{46,48}, C-LTMRs^{46,49}, CGRP⁺ neurons^{50,51} (containing six transcriptionally discrete subtypes), Mrgprd⁺ polymodal nociceptors^{46,52-54,} proprioceptors^{55,56}, Sst⁺ pruriceptors (Somatostatin/Nppb⁺)^{57,58}, cold sensitive thermoceptors^{50,59,60}, as well as two main classes of support cells (Endothelial and Schwann cells). It was noted that a transcriptionally distinct cluster uniquely corresponding to Merkel cell-associated Aβ SA1-LTMRs was not detected. However, based on bulk RNA-seq analysis of genetically defined and FACS-purified LTMR subtypes, Aβ SA1-LTMRs harbor transcriptomes bearing striking resemblance to Aβ Field-LTMRs⁴⁶; therefore, these two Aβ LTMR subtypes are likely embedded within the same cluster in the tSNE plot. It was confirmed that marker genes for each of the sensory neuron subtypes are expressed in subsets of DRG neurons and noted that the relative proportions of certain sensory neuron subtypes varied across ganglia located at different axial levels (FIGS. 7A-7D). Moreover, the somatosensory neuron subtypes identified in this adult DRG analysis are remarkably similar to those identified in scRNA-seq analysis of 5,556 somatosensory neurons obtained from adult trigeminal ganglia (TG) (FIGS. 8A-8D). The cell types identified by the scRNA-seq findings are largely consistent with previously published adult DRG/TG scRNA-seq data sets^{16,17,19,61,62} Exclusion Criteria: As a first quality control filter, individual cells were removed from the data set if they had fewer than 1000 discovered genes, fewer than 1000 UMI or greater than 5% reads mapping to mitochondrial genes (several data sets use a 10% threshold for this parameter and is indicated in the respective figures). Preparing single cell suspensions of DRG/TG sensory neurons often results in a population non-neuronal/neuronal doublets. To circumvent this, individual cells were defined as showing expression of Schwann cell markers (Sox2 or Ednrb) and neuronal markers as neurons that did not resolve into single cells during the dissociation process. Cells matching these criteria were removed before performing subsequent analysis and this analysis was applied to all data sets presented in this study. Lastly, for simplicity, most displays exclude non-neuronal cells (Schwann and endothelial). Generally, it was found that <10% of cells in any given data set were classified as non-neuronal. General analysis parameters: Raw UMI counts were normalized to 10,000 UMIs per cell. Highly variable genes were calculated using the FindVariableGenes function with mean.function=ExpMean, dispersion.function=Log VMR, x.low.cutoff=0, and y.cutoff=0.5. PCA/tSNE analysis were used for dimensionality reduction and elbow plots were generated to determine which principal components to include in the analysis. This corresponded to roughly the first 20 principle components. Canonical correlation analysis (CCA) and matching of cell types through development was performed as previously described²⁷. Identification of differentially expressed genes: Differential gene expression analysis was performed on all expressed genes using the FindMarker function in Seurat using the Wilcoxon-Rank Sum test and a pseudocount of 0.001 was added to each gene to prevent infinite values. P-values <10⁻³²² were defined as 0 as the R-environment does not handle numbers <10⁻³²². Each identified cell type was compared against an outgroup which corresponded to all other cells in the dataset at the respective timepoint. All genes identified were spot checked by overlaying the expression levels on the tSNE plot to ensure the computational method was faithfully identifying genes with the prescribed features. For subtype specific gene expression analysis, subtype specific genes were first defined using the littermate control mice as knockout mice were not always available on pure C57/Bl6 background. The subtype specific genes identified in littermate control mice was nearly identical to those observed in C57/Bl6 control animals. Of the top 100 subtype-specific genes, 50 were randomly selected from this group and compared to the knockout controls. 50 expression matched genes that were not included in the subtype-specific gene list were selected as the randomized control genes. Monocle 3 analysis (for E11 trajectory analysis). The Monocle 3 workflow was performed in a similar fashion as previously described²⁵. In brief the Monocle 3 pipeline offers several key advantages, described here briefly. Firstly, this pipeline allows for the generation of trajectories over potentially discontinuous underlying data. This is first accomplished by performing dimensionality reduction with the recently proposed UMAP algorithm²⁰, instead of tSNE. Notably, UMAP provides comparable visualization quality to tSNE and UMAP also performs better at preserving global relationships, which is a noted shortcoming of the tSNE algorithm. Furthermore, the UMAP algorithm is more efficient [O(N)] compared to tSNE [N log(N)] making UMAP a more computationally friendly option for large datasets, as those used in this study. The UMAP parameters used in this study are comparable to those previously applied²⁵ (reduction.use=“PCA”, max.dim=2 L, neighbors=50, min_dist=0.1, cosine distance metric). It has been noted similar parameters have been used to finely resolve subtrajectories²⁵ and therefore it was argued that these parameters provide greatest sensitivity for identifying branches, if they exist, within out dataset. STITCH analysis. Although UMAP provides an advance in gene expression based trajectory inference, more complex changes in gene expression space, as is observed often in development⁶³ continue to provide a significant challenge to identifying underlying trajectories. A recently proposed algorithm, STITCH, described in⁶³ provides an alternative strategy, which is described here in brief. Instead of projecting all the data into a single low-dimensional space, STITCH assembles a manifold that is defined by a series of independent PCA subspaces corresponding to each individual time point with nodes representing cells and edges linking transcriptionally similar cells in a low-dimensional space. This allows for connections between cells to be identified even if cells are optimally described by differing underlying PC subspaces. From here, each cell in timepoint tᵢ, where i∈(E11.5, E12.5, E15.5, P0, P5, Adult) forms an outgoing edge from tᵢ→tᵢ and tᵢ→tᵢ₋₁, ∀i∈(timepoints) where all cells are projected into the PC subspace defined by tᵢ alone. In essence, edges connect each cell to its closet transcriptional neighbor within a timepoint and the preceding timepoint. Edges are then subjected to local neighborhood restriction such that an outgoing edge from a cell was maintained if its neighbors were at most 3-fold as far as the cell's closest neighbor. To avoid spurious connections that may form, edges were next subjected to a global neighborhood restriction where edges are maintained if they were below the average edge distance across all cells between time points (tᵢ, tᵢ₋₁) or within 1 standard deviation of the average edge distance within the timepoint. The graph was further reduced by retaining at most 20 mutual nearest neighbor edges.

1.

Cloning, Production, Purification, Concentration and Quality Control of Adeno-Associated Virus (AAV).

AAV backbones were generated using standing cloning and molecular biology techniques. The following sequences were used for shRNAs: Luciferase (GCGCGATAGCGCTAATAATTT (SEQ ID NO: 1)), Pou4f3 (TATCCCTTGGAGAAAAGCCTTGTT (SEQ ID NO: 2)). AAVs included GFP, tagged with hemagglutinin (TAC CCATACGATGTTCCAGATTACGCT (SEQ ID NO: 3)) as a reporter to monitor infectivity. Each individual preparation of AAV (2/9) and (2/PHP.S⁶⁴) were produced by transient transfection of pRC9, pHelper, and AAV-genome plasmid into 6-12 T225 flasks of HEK 293T cells. Viral media was collected and replaced at 72 hours. 293T cells and a second round of viral media were collected at 120 hours post transfection. AAVs were extracted from cell pellets using Salt Active Nuclease (Articzymes) in 40 mM Tris, 500 mM NaCl and 2 mM MgCl₂ pH8 (SAN buffer). AAV in supernatant were precipitated with 8% PEG/500 mM NaCl and resuspended in SAN buffer. Viral suspensions were loaded onto an iodixanol gradient (OptiPrep) and subsequently concentrated using Amicon filters with a 100 kD cutoffs to a volume of 25-30 uL (1×PBS+0.001% F-68) per 6 T225-flasks transfected. Viral titers were normalized to 1e10¹⁴ vg/mL and stored at −80C in 5-10 uL aliquots. AAVs (2/9) were injected intraperitoneally (IP) into postnatal day 0 pups. Pups transiently anesthetized by hypothermia and beveled pipettes were used to deliver 10¹² viral genomes in a volume of 10 uL (0.01% Fast Green, 1×PBS). After mice were injected, they were returned to ambient temperature and upon regaining full mobility were cross fostered with nursing CD1 females. Approximately seven days after transduction, DRGs were extracted for subsequent experimental analysis. Upon dissecting, all DRGs were visualized and monitored for GFP expression. For behavioral experiments, a minimum of 10¹² viral genomes of AAV (2/PHP.S) were delivered to P21 mice via intravenous injection (retroorbital vein).

Immunostaining Analysis.

DRG: For immunostaining analysis, mice (P28-42) were anesthetized with isoflurane and transcardially perfused with 10 mL of 1×PBS (with Heparin) followed by 10 mL of 1×PBS/4% paraformaldehyde at room temperature. Spinal columns were then removed and rinsed in 1×PBS and then cryoprotected overnight in 1×PBS/30% sucrose at 4° C., then embedded in NEG50 and stored at −70° C. For cryosectioning, tissue blocks were equilibrated to −20° C. for 1 hour and then sectioned onto glass slides at a thickness of 20-25 μm. Slides were stored at −70° C. until ready for staining. Slides with sections were taken from freezers and immediately placed into 1×PBS and washed 3× with 1×PBS for 5 minutes each at room temperature. Tissue was blocked using 1×PBS/5% Normal donkey serum/0.05% Triton X-100 for 1 hour at room temperature. Tissue was then washed with 1×PBS 3×5 minutes each at room temperature. Tissue was then incubated in primary antibody (Rabbit Anti-NeuN, Millipore: MAB377, 1:1000. Goat Anti-mCherry/tdTomato, CederLane: AB0040-200, 1:1000) in 1×PBS/5% Normal donkey serum/0.05% Triton X-100 overnight at 4° C. Tissue were washed in 1×PBS 3× for 5 minutes at room temperature followed by secondary antibody (Donkey Anti-Rabbit 488, 1:1000; Donkey Anti-Goat, 1:1000) diluted in 1×PBS/5% Normal donkey serum/0.05% Triton X-100 for 1 hour at room temperature. Lastly, tissue was washed in 1×PBS 3× for 5 minutes at room temperature followed by application of mounting media and glass coverslip. Skin sections. Skin sections were immunostained as described for DRG sections with the following differences: Section thickness was 55-60 μm. Primary antibodies used were (Chicken Anti-GFP, Ayes: GFP-1020, 1:1000. Goat Anti-mCherry/tdTomato, CederLane: AB0040-200, 1:1000, Rabbit Anti-CGRP, Immunostar: 24112, 1:1000). All images were obtained as z-stacks using a Zeiss LSM 700 confocal microscope using a 10× or 20× objective.

Two-Plate Temperature Choice Assay.

Animals were habituated to the behavioral apparatus for 30 minutes prior to experimental analysis. Animals were placed into the center of two identical chambers with one chamber randomly set to 30° C. and the other to the test temperature indicated. Animals were recorded as they freely explored the arena while automatic tracking software was used to track animals over a 5-minute period. Time spent in each temperature chamber was quantified as a fraction of total time tested and one temperature was tested per day.

RNA Isolation, Reverse Transcription, and qRT-PCR.

DRGs were dissected as described above; however, instead of subjecting ganglia to dissociation, they were directly lysed by gentle agitation in Trizol at room temperature for 10 minutes. The RNeasy Mini (Qiagen) kit was used according to manufacturer's instructions to purify DNA-free RNA. RNA was converted to cDNA using 200-250 ng of RNA with the High-capacity cDNA reverse transcription kit (Themofisher). qRT-PCR was performed with technical triplicates and mapped back to relative RNA concentrations using a standard curve built from a serial dilution of cDNA. Data were collected using the LightCycler 480 SYBR Green I Master mix (Roche) on a QuantStudio 3 qPCR machine (Applied Biosystems).

Statistics and Reproducibility.

For all scRNA-seq data shown all individual cells for the labeled cell type are shown with no downsampling or subsetting implemented unless explicitly indicated. Differential and comparative gene expression analysis were conducted using a two-sided Wilcoxon rank-sum test with Bonferroni correct p-values. Immunostaining and cell counting comparisons were done using a two-sided t-test. Behavioral analysis was compared using a two-way ANOVA followed by a Tukey's post-hoc test. All scRNA-seq samples were derived from n=2 biologically independent samples with the exception of the adult (P28-42) sample which was derived from n=6 biologically independent samples. The follow sample sizes (cell numbers) for each cell type and samples sizes for other analyses are as follows: FIG. 1A Adult: 257 Aβ Field LTMR cells, 273 Aβ RA LTMR cells, 182 Aδ LTMR cells, 1554 C-LTMR cells, 1440 CGRP-α cells, 850 CGRP-ε cells, 270 CGRP-η cells, 705 CGRP-γ cells, 758 CGRP-θ cells, 333 CGRP-ζ cells, 2817 Mrgprd cells, 234 Proprioceptors cells, 761 Sst cells, 488 Cold Thermoceptors cells, Postnatal day 5 209 Aβ Field LTMR cells, 297 Aβ RA LTMR cells, 237 Aδ LTMR cells, 1392 C-LTMR cells, 445 CGRP-α cells, 473 CGRP-ε cells, 153 CGRP-η cells, 334 CGRP-γ cells, 640 CGRP-θ cells, 243 CGRP-ζ cells, 3019 Mrgprd cells, 104 Proprioceptors cells, 787 Sst cells, 405 Cold Thermoceptors cells, Postnatal day 0 257 Aβ Field LTMR cells, 273 Aβ RA LTMR cells, 182 Aδ LTMR cells, 1554 C-LTMR cells, 1440 CGRP-α cells, 850 CGRP-ε cells, 270 CGRP-η cells, 705 CGRP-γ cells, 758 CGRP-θ cells, 333 CGRP-ζ cells, 2817 Mrgprd cells, 234 Proprioceptors cells, 761 Sst cells, 488 Cold Thermoceptors cells, Postnatal day 0 214 Aβ Field LTMR cells, 163 Aβ RA LTMR cells, 165 Aδ LTMR cells, 739 C-LTMR cells, 284 CGRP-α cells, 188 CGRP-ε cells, 122 CGRP-η cells, 216 CGRP-γ cells, 359 CGRP-θ cells, 122 CGRP-ζ cells, 1704 Mrgprd cells, 103 Proprioceptors cells, 397 Sst cells, 284 Cold Thermoceptors cells, Embryonic Day 15.5 61 Aβ Field LTMR cells, 33 Aβ RA LTMR cells, 96 Aδ LTMR cells, 383 C-LTMR cells, 144 CGRP-α cells, 45 CGRP-ε cells, 26 CGRP-η cells, 97 CGRP-γ cells, 208 CGRP-θ cells, 63 CGRP-ζ cells, 670 Mrgprd cells, 40 Proprioceptors cells, 61 Sst cells, 128 Cold Thermoceptors cells, 3196 unlabeled cells, Embryonic Day 12.5 30 Aβ Field LTMR cells, 20 Aβ RA LTMR cells, 30 Aδ LTMR cells, 122 C-LTMR cells, 57 CGRP-α cells, 87 CGRP-ε cells, 48 CGRP-η cells, 60 CGRP-γ cells, 9 CGRP-θ cells, 37 CGRP-ζ cells, 555 Mrgprd cells, 37 Proprioceptors cells, 24 Sst cells, 105 Cold Thermoceptors cells, 7909 unlabeled cells; FIG. 1B Embryonic Day 11.5 1951 Unspecialized sensory neuron, 5402 Sensory neuron progenitor, 2781 Neural crest progenitor; FIG. 1C n=3 biologically independent samples; FIG. 1D 1951 unspecialized sensory neuron, 257 Aβ Field LTMR cells, 273 Aβ RA LTMR cells, 182 Aδ LTMR cells, 1554 C-LTMR cells, 1440 CGRP-α cells, 850 CGRP-ε cells, 270 CGRP-η cells, 705 CGRP-γ cells, 758 CGRP-θ cells, 333 CGRP-ζ cells, 2817 Mrgprd cells, 234 Proprioceptors cells, 761 Sst cells, 488 Cold Thermoceptors cells; FIGS. 2A-2B 696 Aβ Field LTMR cells, 734 Aβ RA LTMR cells, 659 Aδ LTMR cells, 3750 C-LTMR cells, 2072 CGRP-α cells, 1503 CGRP-ε cells, 555 CGRP-η cells, 1377 CGRP-γ cells, 1895 CGRP-θ cells, 743 CGRP-ζ cells, 7498 Mrgprd cells, 462 Proprioceptors cells, 1733 Sst cells, 1246 Cold Thermoceptors cells, 1951 unspecialized sensory neurons, 14982 cells with unmatched identity; FIG. 2c 1951 unspecialized sensory neurons, 257 Aβ Field LTMR cells, 273 Aβ RA LTMR cells, 182 Aδ LTMR cells, 1554 C-LTMR cells, 1440 CGRP-α cells, 850 CGRP-ε cells, 270 CGRP-11 cells, 705 CGRP-γ cells, 758 CGRP-θ cells, 333 CGRP-ζ cells, 2817 Mrgprd cells, 234 Proprioceptors cells, 761 Sst cells, 488 Cold Thermoceptors cells; FIG. 3a 10321 cells from E11.5; FIG. 3c 159 Aβ Field LTMR cells, 385 Aβ RA LTMR cells, 203 Aδ LTMR cells, 1827 C-LTMR cells, 441 CGRP-α cells, 334 CGRP-ε cells, 348 CGRP-η cells, 417 CGRP-γ cells, 1665 CGRP-θ cells, 196 CGRP-ζ cells, 3666 Mrgprd cells, 185 Proprioceptors cells, 802 Sst cells, 717 Cold Thermoceptors cells; FIG. 3e n=3 biologically independent samples; FIG. 4a,c control/knockout 776/435 Aβ Field LTMR cells, 728/1114 Aβ RA LTMR cells, 667/927 Aδ LTMR cells, 2928/2486 C-LTMR cells, 478/656 CGRP-α cells, 990/582 CGRP-ε cells, 721/589 CGRP-η cells, 711/540 CGRP-γ cells, 1845/2381 CGRP-θ cells, 417/230 CGRP-ζ cells, 5556/7508 Mrgprd cells, 446/654 Proprioceptors cells, 1747/1460 Sst cells, 493/675 Cold Thermoceptors cells; FIG. 4b,d control/knockout 191/254 Aβ Field LTMR cells, 246/332 Aβ RA LTMR cells, 170/236 Aδ LTMR cells, 917/800 C-LTMR cells, 706/545 CGRP-α cells, 495/365 CGRP-ε cells, 279/330 CGRP-η cells, 559/429 CGRP-γ cells, 907/605 CGRP-θ cells, 292/341 CGRP-ζ cells, 1977/2960 Mrgprd cells, 123/213 Proprioceptors cells, 724/835 Sst cells, 427/392 Cold Thermoceptors cells; FIG. 5a-c 159 in control 342 Aβ Field LTMR cells, 122 Aβ RA LTMR cells, 413 Aδ LTMR cells, 783 C-LTMR cells, 363 CGRP-α cells, 314 CGRP-ε cells, 320 CGRP-η cells, 418 CGRP-γ cells, 460 CGRP-θ cells, 352 CGRP-ζ cells, 1162 Mrgprd cells, 368 Proprioceptors cells, 149 Sst cells, 442 Cold Thermoceptors cells, in NGF^{-/-}; Bax^{-/-} 82 Aβ Field LTMR cells, 162 Aβ RA LTMR cells, 124 Aδ LTMR cells, 395 Proprioceptors, 2558 ClusterA cells, 1878 ClusterB cells, 362, ClusterC cells, 1461 ClusterD cells, 714 ClusterE cells; FIG. 6A 257 Aβ Field LTMR cells, 273 Aβ RA LTMR cells, 182 Aδ LTMR cells, 1554 C-LTMR cells, 1440 CGRP-α cells, 850 CGRP-ε cells, 270 CGRP-η cells, 705 CGRP-γ cells, 758 CGRP-θ cells, 333 CGRP-ζ cells, 2817 Mrgprd cells, 234 Proprioceptors cells, 761 Sst cells, 488 Cold Thermoceptors cells; FIG. 6B 209 Aβ Field LTMR cells, 297 Aβ RA LTMR cells, 237 Aδ LTMR cells, 1392 C-LTMR cells, 445 CGRP-α cells, 473 CGRP-ε cells, 153 CGRP-η cells, 334 CGRP-γ cells, 640 CGRP-θ cells, 243 CGRP-ζ cells, 3019 Mrgprd cells, 104 Proprioceptors cells, 787 Sst cells, 405 Cold Thermoceptors cells; FIG. 6C 257 Aβ Field LTMR cells, 273 Aβ RA LTMR cells, 182 Aδ LTMR cells, 1554 C-LTMR cells, 1440 CGRP-α cells, 850 CGRP-ε cells, 270 CGRP-η cells, 705 CGRP-γ cells, 758 CGRP-θ cells, 333 CGRP-ζ cells, 2817 Mrgprd cells, 234 Proprioceptors cells, 761 Sst cells, 488 Cold Thermoceptors cells; FIG. 6D 214 Aβ Field LTMR cells, 163 Aβ RA LTMR cells, 165 Aδ LTMR cells, 739 C-LTMR cells, 284 CGRP-α cells, 188 CGRP-ε cells, 122 CGRP-η cells, 216 CGRP-γ cells, 359 CGRP-O cells, 122 CGRP-ζ cells, 1704 Mrgprd cells, 103 Proprioceptors cells, 397 Sst cells, 284 Cold Thermoceptors cells; FIG. 6D 61 Aβ Field LTMR cells, 33 Aβ RA LTMR cells, 96 Aδ LTMR cells, 383 C-LTMR cells, 144 CGRP-α cells, 45 CGRP-ε cells, 26 CGRP-η cells, 97 CGRP-γ cells, 208 CGRP-O cells, 63 CGRP-ζ cells, 670 Mrgprd cells, 40 Proprioceptors cells, 61 Sst cells, 128 Cold Thermoceptors cells; FIG. 6E 30 Aβ Field LTMR cells, 20 Aβ RA LTMR cells, 30 Aδ LTMR cells, 122 C-LTMR cells, 57 CGRP-α cells, 87 CGRP-ε cells, 48 CGRP-η cells, 60 CGRP-γ cells, 9 CGRP-O cells, 37 CGRP-ζ cells, 555 Mrgprd cells, 37 Proprioceptors cells, 24 Sst cells, 105 Cold Thermoceptors cells; FIG. 6F Mature/P5 merge, P5/P0 merge, E15.5/P0 merge, E15.5/E12.5 merge; FIG. 7A,B n=15 biologically independent sections for each in situ at each axial level, FIG. 8A-C 293 Aβ RA LTMR cells, 106 Aδ LTMR cells, 408 C-LTMR cells, 225 CGRP-α cells, 595 CGRP-ε cells, 127 CGRP-η cells, 329 CGRP-γ cells, 199 CGRP-O cells, 96 CGRP-ζ cells, 1103 Mrgprd cells, 131 cells, 656 Cold Thermoceptors cells; FIG. 9A-E 257 Aβ Field LTMR cells, 273 Aβ RA LTMR cells, 182 Aδ LTMR cells, 1554 C-LTMR cells, 1440 CGRP-α cells, 850 CGRP-ε cells, 270 CGRP-η cells, 705 CGRP-γ cells, 758 CGRP-O cells, 333 CGRP-ζ cells, 2817 Mrgprd cells, 234 Proprioceptors cells, 761 Sst cells, 488 Cold Thermoceptors cells, 1951 Unspecialized sensory neuron (USN), 5402 Sensory neuron progenitor (SNP), 2781 Neural crest progenitor (NCP), FIG. 10A 696 Aβ Field LTMR cells, 734 Aβ RA LTMR cells, 659 Aδ LTMR cells, 3750 C-LTMR cells, 2072 CGRP-α cells, 1503 CGRP-ε cells, 555 CGRP-η cells, 1377 CGRP-γ cells, 1895 CGRP-O cells, 743 CGRP-ζ cells, 7498 Mrgprd cells, 462 Proprioceptors cells, 1733 Sst cells, 1246 Cold Thermoceptors cells, 1951 unspecialized sensory neurons, 14982 cells with unmatched identity; FIG. 10B 257 Aβ Field LTMR cells, 273 Aβ RA LTMR cells, 182 Aδ LTMR cells, 1554 C-LTMR cells, 1440 CGRP-α cells, 850 CGRP-ε cells, 270 CGRP-η cells, 705 CGRP-γ cells, 758 CGRP-O cells, 333 CGRP-ζ cells, 2817 Mrgprd cells, 234 Proprioceptors cells, 761 Sst cells, 488 Cold Thermoceptors cells; FIG. 10C n=3 biologically independent samples, FIG. 11A,B Adult: 257 Aβ Field LTMR cells, 273 Aβ RA LTMR cells, 182 Aδ LTMR cells, 1554 C-LTMR cells, 1440 CGRP-α cells, 850 CGRP-ε cells, 270 CGRP-η cells, 705 CGRP-γ cells, 758 CGRP-θ cells, 333 CGRP-ζ cells, 2817 Mrgprd cells, 234 Proprioceptors cells, 761 Sst cells, 488 Cold Thermoceptors cells, Postnatal day 5 209 Aβ Field LTMR cells, 297 Aβ RA LTMR cells, 237 Aδ LTMR cells, 1392 C-LTMR cells, 445 CGRP-α cells, 473 CGRP-ε cells, 153 CGRP-η cells, 334 CGRP-γ cells, 640 CGRP-θ cells, 243 CGRP-t cells, 3019 Mrgprd cells, 104 Proprioceptors cells, 787 Sst cells, 405 Cold Thermoceptors cells, Postnatal day 0 257 Aβ Field LTMR cells, 273 Aβ RA LTMR cells, 182 Aδ LTMR cells, 1554 C-LTMR cells, 1440 CGRP-α cells, 850 CGRP-ε cells, 270 CGRP-η cells, 705 CGRP-γ cells, 758 CGRP-θ cells, 333 CGRP-ζ cells, 2817 Mrgprd cells, 234 Proprioceptors cells, 761 Sst cells, 488 Cold Thermoceptors cells, Postnatal day 0 214 Aβ Field LTMR cells, 163 Aβ RA LTMR cells, 165 Aδ LTMR cells, 739 C-LTMR cells, 284 CGRP-α cells, 188 CGRP-ε cells, 122 CGRP-η cells, 216 CGRP-γ cells, 359 CGRP-θ cells, 122 CGRP-ζ cells, 1704 Mrgprd cells, 103 Proprioceptors cells, 397 Sst cells, 284 Cold Thermoceptors cells, Embryonic Day 15.5 61 Aβ Field LTMR cells, 33 Aβ RA LTMR cells, 96 Aδ LTMR cells, 383 C-LTMR cells, 144 CGRP-α cells, 45 CGRP-ε cells, 26 CGRP-η cells, 97 CGRP-γ cells, 208 CGRP-θ cells, 63 CGRP-ζ cells, 670 Mrgprd cells, 40 Proprioceptors cells, 61 Sst cells, 128 Cold Thermoceptors cells, Embryonic Day 12.5 30 Aβ Field LTMR cells, 20 Aβ RA LTMR cells, 30 Aδ LTMR cells, 122 C-LTMR cells, 57 CGRP-α cells, 87 CGRP-ε cells, 48 CGRP-η cells, 60 CGRP-γ cells, 9 CGRP-θ cells, 37 CGRP-ζ cells, 555 Mrgprd cells, 37 Proprioceptors cells, 24 Sst cells, 105 Cold Thermoceptors cells, FIG. 12A n=3 biologically independent samples for the Avil in situ, control/knockout 776/435 Aβ Field LTMR cells, 728/1114 Aβ RA LTMR cells, 667/927 Aδ LTMR cells, 2928/2486 C-LTMR cells, 478/656 CGRP-α cells, 990/582 CGRP-ε cells, 721/589 CGRP-η cells, 711/540 CGRP-γ cells, 1845/2381 CGRP-θ cells, 417/230 CGRP-ζ cells, 5556/7508 Mrgprd cells, 446/654 Proprioceptors cells, 1747/1460 Sst cells, 493/675 Cold Thermoceptors cells; FIG. 12B n=3 biologically independent samples for the in situ, control/knockout 191/254 Aβ Field LTMR cells, 246/332 Aβ RA LTMR cells, 170/236 Aδ LTMR cells, 917/800 C-LTMR cells, 706/545 CGRP-α cells, 495/365 CGRP-ε cells, 279/330 CGRP-η cells, 559/429 CGRP-γ cells, 907/605 CGRP-θ cells, 292/341 CGRP-ζ cells, 1977/2960 Mrgprd cells, 123/213 Proprioceptors cells, 724/835 Sst cells, 427/392 Cold Thermoceptors cells; FIG. 13E, 13F tSNE plots represent 257 Aβ Field LTMR cells, 273 Aβ RA LTMR cells, 182 Aδ LTMR cells, 1554 C-LTMR cells, 1440 CGRP-α cells, 850 CGRP-ε cells, 270 CGRP-η cells, 705 CGRP-γ cells, 758 CGRP-θ cells, 333 CGRP-ζ cells, 2817 Mrgprd cells, 234 Proprioceptors cells, 761 Sst cells, 488 Cold Thermoceptors cells; FIG. 13H n=3 biologically independent samples for the immunostaining, FIG. 13J n=3 biologically independent samples for the immunostaining, FIG. 13K n=3 biologically independent samples for the immunostaining and the in situ, FIG. 14A n=3 biologically independent samples; FIG. 14B-f 112/231 Aβ Field LTMR cells, 155/301 Aβ RA LTMR cells, 124/132 Aδ LTMR cells, 1124/1111 C-LTMR cells, 225/231 CGRP-α cells, 369/239 CGRP-ε cells, 105/76 CGRP-η cells, 235/117 CGRP-γ cells, 573/674 CGRP-θ cells, 209/110 CGRP-ζ cells, 2174/2345 Mrgprd cells, 95/170 Proprioceptors cells, 701/721 Sst cells, 175/201 Cold Thermoceptors cells FIG. 14G n=8 biologically independent samples for luciferase shRNA, n=8 biological samples for Pou4f3 shRNA; FIG. 14H n=3 biologically independent samples; FIG. 15A control 342 Aβ Field LTMR cells, 122 Aβ RA LTMR cells, 413 Aδ LTMR cells, 783 C-LTMR cells, 363 CGRP-α cells, 314 CGRP-ε cells, 320 CGRP-η cells, 418 CGRP-γ cells, 460 CGRP-θ cells, 352 CGRP-ζ cells, 1162 Mrgprd cells, 368 Proprioceptors cells, 149 Sst cells, 442 Cold Thermoceptors cells, in NGF^{-/-}; Bax^{-/-} 82 Aβ Field LTMR cells, 162 Aβ RA LTMR cells, 124 Aδ LTMR cells, 395 Proprioceptors, 2558 ClusterA cells, 1878 ClusterB cells, 362, ClusterC cells, 1461 ClusterD cells, 714 ClusterE cells; FIG. 15C n=3 biologically independent samples for each in situ.

Data Availability.

Sequence data of this study have been deposited with accession code GSE139088. The data is also available for browsing and analysis via the HTML interface at kleintools.hms.harvard.edu/tools/springViewer_1_6_dev.html?datasets/Sharma2019/all.

Code Availability.

The computational code used in the study is available at GitHub (github.com/wagnerde) or upon request.

### REFERENCES FOR EXAMPLE 1

1 Abraira, V. E. & Ginty, D. D. The sensory neurons of touch. Neuron 79, 618-639, doi:10.1016/j.neuron.2013.07.051 (2013).
2 Julius, D. TRP channels and pain. Annu Rev Cell Dev Biol 29, 355-384, doi:10.1146/annurev-cellbio-101011-155833 (2013).
3 Basbaum, A. I., Bautista, D. M., Scherrer, G. & Julius, D. Cellular and molecular mechanisms of pain. Cell 139, 267-284, doi:10.1016/j.cell.2009.09.028 (2009).
4 Julius, D. & Basbaum, A. I. Molecular mechanisms of nociception. Nature 413, 203-210, doi:10.1038/35093019 (2001).
5 Le Douarin, N. The neural crest. (Cambridge University Press, 1982).
6 Anderson, D. J. Lineages and transcription factors in the specification of vertebrate primary sensory neurons. Curr Opin Neurobiol 9, 517-524, doi:10.1016/50959-4388(99)00015-X (1999).
7 Marmigere, F. & Ernfors, P. Specification and connectivity of neuronal subtypes in the sensory lineage. Nat Rev Neurosci 8, 114-127, doi:10.1038/nrn2057 (2007).
8 Lallemend, F. & Ernfors, P. Molecular interactions underlying the specification of sensory neurons. Trends Neurosci 35, 373-381, doi:10.1016/j.tins.2012.03.006 (2012).
9 Kitao, Y., Robertson, B., Kudo, M. & Grant, G. Neurogenesis of subpopulations of rat lumbar dorsal root ganglion neurons including neurons projecting to the dorsal column nuclei. J Comp Neurol 371, 249-257, doi:10.1002/(SICI)1096-9861(19960722)371:2<249::AID-CNE5>3.0.CO;2-2 (1996).
10 Hasegawa, H., Abbott, S., Han, B. X., Qi, Y. & Wang, F. Analyzing somatosensory axon projections with the sensory neuron-specific Advillin gene. J Neurosci 27, 14404-14414, doi:10.1523/JNEUROSCI.4908-07.2007 (2007).
11 Ozaki, S. & Snider, W. D. Initial trajectories of sensory axons toward laminar targets in the developing mouse spinal cord. J Comp Neurol 380, 215-229 (1997).
12 Mimics, K. & Koerber, H. R. Prenatal development of rat primary afferent fibers: II. Central projections. J Comp Neurol 355, 601-614, doi:10.1002/cne.903550409 (1995).
13 Mimics, K. & Koerber, H. R. Prenatal development of rat primary afferent fibers: I. Peripheral projections. J Comp Neurol 355, 589-600, doi:10.1002/cne.903550408 (1995).
14 Woodbury, C. J., Ritter, A. M. & Koerber, H. R. Central anatomy of individual rapidly adapting low-threshold mechanoreceptors innervating the “hairy” skin of newborn mice: early maturation of hair follicle afferents. J Comp Neurol 436, 304-323 (2001).
15 Woodbury, C. J. & Koerber, H. R. Widespread projections from myelinated nociceptors throughout the substantia gelatinosa provide novel insights into neonatal hypersensitivity. J Neurosci 23, 601-610 (2003).
16 Zeisel, A. et al. Molecular Architecture of the Mouse Nervous System. Cell 174, 999-1014 e1022, doi:10.1016/j.cell.2018.06.021 (2018).
17 Usoskin, D. et al. Unbiased classification of sensory neuron types by large-scale single-cell RNA sequencing. Nat Neurosci 18, 145-153, doi:10.1038/nn.3881 (2015).
18 Zheng, Y. et al. Deep Sequencing of Somatosensory Neurons Reveals Molecular Determinants of Intrinsic Physiological Properties. Neuron 103, 598-616 e597, doi:10.1016/j.neuron.2019.05.039 (2019).
19 Nguyen, M. Q., Wu, Y., Bonilla, L. S., von Buchholtz, L. J. & Ryba, N. J. P. Diversity amongst trigeminal neurons revealed by high throughput single cell sequencing. PLoS One 12, e0185543, doi:10.1371/journal.pone.0185543 (2017).
20 McInnes, L., Healy, J. & Melville, J. UMAP: Uniform manifold approximation and projection for dimension reduction. arXiv, 1-51 (2018).
21 Kim, J., Lo, L., Dormand, E. & Anderson, D. J. SOX10 maintains multipotency and inhibits neuronal differentiation of neural crest stem cells. Neuron 38, 17-31 (2003).
22 Britsch, S. et al. The transcription factor Sox10 is a key regulator of peripheral glial development. Genes Dev 15, 66-78 (2001).
23 Ma, Q., Fode, C., Guillemot, F. & Anderson, D. J. Neurogenin1 and neurogenin2 control two distinct waves of neurogenesis in developing dorsal root ganglia. Genes Dev 13, 1717-1728 (1999).
24 Zurborg, S. et al. Generation and characterization of an Advillin-Cre driver mouse line. Mol Pain 7, 66, doi:10.1186/1744-8069-7-66 (2011).
25 Cao, J. et al. The single-cell transcriptional landscape of mammalian organogenesis. Nature 566, 496-502, doi:10.1038/s41586-019-0969-x (2019).
26 Blanchard, J. W. et al. Selective conversion of fibroblasts into peripheral sensory neurons. Nat Neurosci 18, 25-35, doi:10.1038/nn.3887 (2015).
27 Mayer, C. et al. Developmental diversification of cortical inhibitory interneurons. Nature 555, 457-462, doi:10.1038/nature25999 (2018).
28 Inoue, K. et al. Runx3 controls the axonal projection of proprioceptive dorsal root ganglion neurons. Nat Neurosci 5, 946-954, doi:10.1038/nn925 (2002).
29 Levanon, D. et al. The Runx3 transcription factor regulates development and survival of TrkC dorsal root ganglia neurons. EMBO J 21, 3454-3463, doi:10.1093/emboj/cdf370 (2002).
30 Chen, C. L. et al. Runx1 determines nociceptive sensory neuron phenotype and is required for thermal and neuropathic pain. Neuron 49, 365-377, doi:10.1016/j.neuron.2005.10.036 (2006).
31 Yoshikawa, M. et al. Coexpression of Runx1 and Runx3 in mechanoreceptive dorsal root ganglion neurons. Dev Neurobiol 73, 469-479, doi:10.1002/dneu.22073 (2013).
32 Lawson, S. N. & Biscoe, T. J. Development of mouse dorsal root ganglia: an autoradiographic and quantitative study. J Neurocytol 8, 265-274 (1979).
33 Lawson, S. N., Caddy, K. W. & Biscoe, T. J. Development of rat dorsal root ganglion neurones. Studies of cell birthdays and changes in mean cell diameter. Cell Tissue Res 153, 399-413, doi:10.1007/bf00229167 (1974).
34 Crowley, C. et al. Mice lacking nerve growth factor display perinatal loss of sensory and sympathetic neurons yet develop basal forebrain cholinergic neurons. Cell 76, 1001-1011 (1994).
35 Patel, T. D., Jackman, A., Rice, F. L., Kucera, J. & Snider, W. D. Development of sensory neurons in the absence of NGF/TrkA signaling in vivo. Neuron 25, 345-357 (2000).
36 Miyamoto, T. et al. Myeloid or lymphoid promiscuity as a critical step in hematopoietic lineage commitment. Dev Cell 3, 137-147 (2002).
37 Hu, M. et al. Multilineage gene expression precedes commitment in the hemopoietic system. Genes Dev 11, 774-785, doi:10.1101/gad.11.6.774 (1997).
38 Orkin, S. H. Diversification of haematopoietic stem cells to specific lineages. Nat Rev Genet 1, 57-64, doi:10.1038/35049577 (2000).
39 Soldatov, R. et al. Spatiotemporal structure of cell fate decisions in murine neural crest. Science 364, doi:10.1126/science.aas9536 (2019).
40 Dasen, J. S., Tice, B. C., Brenner-Morton, S. & Jessell, T. M. A Hox regulatory network establishes motor neuron pool identity and target-muscle connectivity. Cell 123, 477-491, doi:10.1016/j.cell.2005.09.009 (2005).
41 Dasen, J. S., Liu, J. P. & Jessell, T. M. Motor neuron columnar fate imposed by sequential phases of Hox-c activity. Nature 425, 926-933, doi:10.1038/nature02051 (2003).
42 Briscoe, J., Pierani, A., Jessell, T. M. & Ericson, J. A homeodomain protein code specifies progenitor cell identity and neuronal fate in the ventral neural tube. Cell 101, 435-445, doi:10.1016/s0092-8674(00)80853-3 (2000).
43 Hoppe, P. S. et al. Early myeloid lineage choice is not initiated by random PU.1 to GATA1 protein ratios. Nature 535, 299-302, doi:10.1038/nature18320 (2016).
44 Wende, H. et al. The transcription factor c-Maf controls touch receptor development and function. Science 335, 1373-1376, doi:10.1126/science.1214314 (2012).
45 Ichikawa, H., Deguchi, T., Nakago, T., Jacobowitz, D. M. & Sugimoto, T. Parvalbumin, calretinin and carbonic anhydrase in the trigeminal and spinal primary neurons of the rat. Brain Res 655, 241-245 (1994).
46 Zheng, Y. et al. Deep sequencing of somatosensory neurons reveals molecular determinants of intrinsic physiological properties. Neuron In Press (2019).
47 Bal, L. et al. Genetic Identification of an Expansive Mechanoreceptor Sensitive to Skin Stroking. Cell 163, 1783-1795, doi:10.1016/j.cell.2015.11.060 (2015).
48 Rutlin, M. et al. The cellular and molecular basis of direction selectivity of Adelta-LTMRs. Cell 159, 1640-1651, doi:10.1016/j.cell.2014.11.038 (2014).
49 Li, L. et al. The functional organization of cutaneous low-threshold mechanosensory neurons. Cell 147, 1615-1627, doi:10.1016/j.cell.2011.11.027 (2011).
50 Kobayashi, K. et al. Distinct expression of TRPM8, TRPA1, and TRPV1 mRNAs in rat primary afferent neurons with adelta/c-fibers and colocalization with trk receptors. J Comp Neurol 493, 596-606, doi:10.1002/cne.20794 (2005).
51 Rosenfeld, M. G. et al. Production of a novel neuropeptide encoded by the calcitonin gene via tissue-specific RNA processing. Nature 304, 129-135 (1983).
52 Dong, X., Han, S., Zylka, M. J., Simon, M. I. & Anderson, D. J. A diverse family of GPCRs expressed in specific subsets of nociceptive sensory neurons. Cell 106, 619-632 (2001).
53 Zylka, M. J., Dong, X., Southwell, A. L. & Anderson, D. J. Atypical expansion in mice of the sensory neuron-specific Mrg G protein-coupled receptor family. Proc Natl Acad Sci USA 100, 10043-10048, doi:10.1073/pnas.1732949100 (2003).
54 Zylka, M. J., Rice, F. L. & Anderson, D. J. Topographically distinct epidermal nociceptive circuits revealed by axonal tracers targeted to Mrgprd. Neuron 45, 17-25, doi:10.1016/j.neuron.2004.12.015 (2005).
55 Arber, S., Ladle, D. R., Lin, J. H., Frank, E. & Jessell, T. M. ETS gene Er81 controls the formation of functional connections between group Ia sensory afferents and motor neurons. Cell 101, 485-498 (2000).
56 de Nooij, J. C., Doobar, S. & Jessell, T. M. Etv1 inactivation reveals proprioceptor subclasses that reflect the level of NT3 expression in muscle targets. Neuron 77, 1055-1068, doi:10.1016/j.neuron.2013.01.015 (2013).
57 Stantcheva, K. K. et al. A subpopulation of itch-sensing neurons marked by Ret and somatostatin expression. EMBO Rep 17, 585-600, doi:10.15252/embr.201540983 (2016).
58 Mishra, S. K. & Hoon, M. A. The cells and circuitry for itch responses in mice. Science 340, 968-971, doi:10.1126/science.1233765 (2013).
59 Bautista, D. M. et al. The menthol receptor TRPM8 is the principal detector of environmental cold. Nature 448, 204-208, doi:10.1038/nature05910 (2007).
60 McKemy, D. D., Neuhausser, W. M. & Julius, D. Identification of a cold receptor reveals a general role for TRP channels in thermosensation. Nature 416, 52-58, doi:10.1038/nature719 (2002).
61 Hockley, J. R. F. et al. Single-cell RNAseq reveals seven classes of colonic sensory neuron. Gut, doi:10.1136/gutjnl-2017-315631 (2018).
62 Li, C. L. et al. Somatosensory neuron types identified by high-coverage single-cell RNA-sequencing and functional heterogeneity. Cell Res 26, 967, doi:10.1038/cr.2016.90 (2016).

### Example 2: Selective Inhibition of Primary Nociceptors to Reduce Pain and Itch

### INTRODUCTION

The perception of pain relies on primary sensory neurons that innervate the skin and other peripheral organs. The current understanding of the mechanisms by which noxious stimuli are detected and conveyed by primary sensory neurons to the central nervous system is remarkably deficient^{1,2}. This has resulted in an innovation gap in developing new therapeutic approaches to pain, leaving few treatment options for prevalent diseases leading to debilitating pain and itch as found in painful diabetic neuropathy (PDN, ˜9,000 cases per 100,000 in the U.S.) or chronic pruritus (7,000 cases per 100,000). The current standard of care for these two disorders alone represents a market of nearly $10B, however the treatments for these, as well as a majority of pain disorders, have remained unchanged for decades. “First-line” treatment options include the anticonvulsants Gabapentin and Pregabalin, which have poor efficacy and serious side effects, while other treatment options involve the alarming use of opioids, contributing to the addiction epidemic. In light of limited treatment options for pain disorders, there is a clear clinical and societal need for understanding the first stage of nociception which will lead to the development of a fundamentally new class of treatment options to manage pain. Described herein is a strategy that combines next-generation genomic technologies and bioinformatics with compound screening to identify novel molecules that selectively inhibit primary nociceptors to reduce pain, circumventing addictive properties of existing pain treatment paradigms. A parallel strategy will be undertaken to identify drugs that reduce chronic itch.

### Preliminary Results

The perception of pain and itch begins with detection of noxious stimuli by primary peripheral sensory neurons called nociceptors or pruriceptors, respectively. The development of all primary somatosensory neuron subtypes have recently been characterized, which include nociceptors and pruriceptors, using single-cell RNA-seq (scRNA-seq)³. Through this analysis, gene expression patterns for six transcriptionally distinct cellular subtypes of nociceptors and two transcriptionally distinct cellular subtypes of pruriceptors have been defined. Furthermore, using computational algorithms, gene expression changes in each nociceptor and pruriceptor subtype throughout development and maturation (FIG. 27) have been defined. Many of the sensory neuron subtypes identified by the scRNA-seq analysis have been largely uncharacterized and represent an unprecedented level of access to neuronal subtype structure, which is the first step of pain and itch processing. This Example now reveals new candidate therapeutic targets in peripheral pain and itch processing pathways.

Here, the knowledge of primary nociceptor and pruriceptor subtypes, and the newly identified genes they express, is used to develop therapeutic approaches to treat pain and chronic itch. In order to identify novel compounds useful for treating pain and chronic itch by silencing primary nociceptors and pruriceptors, the follow experiments can be conducted:

Experiment 1: Identify G_{ai/o}-coupled GPCRs expressed in specific sensory neuron subtypes that, upon activation, suppress pain and itch processing. A series of established bioinformatics analysis pipelines, behavioral phenotyping, and confirmation of conservation in human tissue samples will be performed.

Experiment 2: Perform high throughput compound screens for molecules that activate subtype restricted G_{ai/o}-coupled GPCRs and therefore block pain and itch signaling. Compound libraries will be screened and hits will be tested in established behavioral and functional assays.

The goal is to identify ligands/agonists of sensory neuron subtype restricted G_{ai/o}-coupled GPCRs that selectively block pain and itch signals emanating from the periphery and subsequently develop those hits into optimal therapeutics for treating pain and chronic itch disorders.

### Experiment 1

Identify G_{ai/o}-coupled GPCRs expressed in specific sensory neuron subtypes that, upon activation, suppress pain and itch processing.

Rationale

GPCRs represent a successful molecular target for modern drug development, with nearly a third of all FDA approved drugs targeting members of this receptor family. Importantly, it is well established that activation of GPCR family members coupled to G_{ai/o} leads to downstream activation of G-protein coupled inwardly rectifying potassium channels (GIRKs) that silence neuronal activity⁴⁻⁶. Moreover, voltage-gated calcium channels necessary for neurotransmitter release from DRG sensory neurons are also inhibited by Gβγ released from G_{ai/o}. Therefore, G_{ai/o} coupled GPCRs are compelling pharmacological targets for silencing the sensory neuron subtypes in which they are expressed. Given that sensory neuron subtypes are responsible for responding to unique types of noxious stimuli, the aim is to identify G_{ai/o} coupled GPCRs that are restricted in expression to one or more subsets of nociceptors and pruriceptors, but not proprioceptors or other sensory neurons subtypes; these will be useful candidate drug targets for selectively silencing the responses to painful stimuli or itch stimuli, thereby reducing pain or itch perception. The ideal GPCR for an agonist compound useful for treating pain or itch will have the following properties: 1) The GPCR is highly expressed in nociceptors, pruriceptors, or combinations of both; 2) The GPCR is coupled to the G_{ai/o} signaling pathway; 3) The GPCR exhibits a conserved pattern of expression between rodent and human DRGs; 4) It is expressed at low levels in other sensory neuron subtypes, peripheral tissues, and brain; 5) Activation of the GPCR attenuates pain or itch perception. Experiment 1 will combine bioinformatic analyses and in vitro and in vivo experiments to identify GPCRs that satisfy these criteria and serve as targets for compound screening and drug development, described in Experiment 2.

1a. Bioinformatic Identification of Subtype-Restricted GPCRs

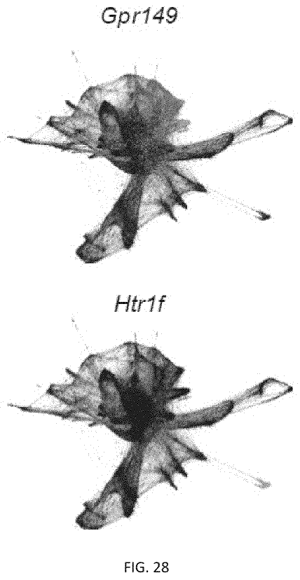

To identify sensory neuron subtype-restricted G_{ai/o} coupled GPCRs, first the scRNA-seq database will be analyzed, and GPCRs with expression profiles restricted to specific subsets of nociceptors and pruriceptors will be bioinformatically identified. The preliminary analysis identified several subtype-restricted GPCRs which are found in all nociceptors, a subset of nociceptors, or pruriceptor subtypes. These GPCRs are undetectable in other somatosensory neuron subtypes (two examples are shown in FIG. 28). Some of these GPCRs have known ligands and are already known to be G_{ai/o}-coupled (e.g, the serotonin receptor Htr1f), whereas others are “orphan” GPCRs and their G protein coupling mechanisms and agonists are unknown (e.g, Gpr149). Which of the identified GPCRs are G_{ai/o}-coupled and thus inhibitory in Experiment 1c will be determined. The extended bioinformatic analysis will identify all of the GPCRs expressed in one or any combination of nociceptors and pruriceptors. This approach will generate a ‘GPCR Candidate Master List’ and the restricted patterns of expression of individual GPCRs in this list will be experimentally confirmed by smRNA-FISH analysis on mouse DRGs. The current GPCR Candidate Master List is shown in FIG. 29, and include the following GPCRs: ADGRA1, ADGRD1, ADGRE5, ADGRF5, ADORA2A, ADORA2B, ADRA2A, ADRA2C, AGTR1A, AGTR1B, AGTRAP, AVPR1A, CALCRL, CELSR2, CESLR3, CHRM1, CRCP, CYSLTR2, DRD1, F2RL1, F2RL2, FZD3, FZD5, FZD8, GALR1, GHSR, GPR35, GPR149, GPR156, GPR173, GPR174, GPR19, GPR4, GRM5, GRM7, HCRTR1, HTR1B, HTR1F, HTR4, HTR5A, LPAR1, LPAR3, MRGPRA1, MRGPRA3, MRGPRA4, MRGPRB4, MRGPRB5, MRGRPD, MRGPRE, MRGPRX1, NPY1R, NYP2R, OGFR, OLFR139, OPN3, OPRK1, OPRM1, OXTR, PROKR2, PTAFR, PTGDR, PTGER1, PTGER2, PTGER3, PTGFR, PTGIR, RAMP3, RHO, S1PR1, S1PR2, S1PR3, SSTR2, VMN1R85, and VMN1R89.

1b. Confirmation of GPCR Expression in Human DRG Tissue and Comparisons to Other Body Regions

A multi-pronged approach will be used to determine whether the GPCRs identified bioinformatically from the dataset of mouse DRG scRNA-Seq are conserved in their expression patterns in human DRGs, and also whether they are expressed at low or undetectable levels in other tissues. It is noteworthy that the degree of conservation of gene expression patterns between mouse and human DRG neurons is believed to be high⁸. As an initial approach, transcriptomic databases will be used to perform bioinformatic cross-species and tissue comparisons. First, expression profiles of the identified GPCRs will be compared to currently published human DRG datasets⁸ that are available to assess the conservation of subtype-restricted GPCRs from mouse to human. As an alternative approach, surgically excised human DRGs will be obtained from the Massachusetts General Hospital and will be sectioned and double smRNA-FISH hybridization will be performed for sensory neuron-subtype marker genes and candidate subtype-restricted GPCRs to evaluate mouse to human conservation.

As an additional bioinformatic analysis, subtype-restricted GPCRs that are confirmed to be conserved in humans will be examined for off-target tissue expression by bioinformatically examining published organism-wide scRNA-seq^{9,10} datasets and evaluating where else the identified subtype-restricted GPCRs are expressed. While some or many of the GPCRs that are expressed in a subset of sensory neurons may also be expressed in other tissues, the Master List will be prioritized to emphasize those GPCRs that show the least amount of expression in non-sensory neuron cell types. Thus, a new, revised GPCR Candidate Master List will be ranked in priority based on patterns of sensory neuron expression, conservation in human, and minimal off target patterns of tissue expression.

1c. G-Protein Signaling Assays to Identify G_{ai/o}-Coupled Receptors

Which of the subtype restricted GPCRs are G_{ai/o}-coupled will be determined. Of those GPCRs listed in FIG. 29, 19 of them are already known to be G_{ai/o}-coupled. To create a standardized assay to test others, each of the most compelling subtype-restricted GPCRs will be fused (both mouse and human versions) to a common ligand binding domain, specifically the extracellular domain (ECD) of the previously published DREADD receptor hM4D, which is activated by the pharmacologically inert compound clozapine-N-oxide (CNO)⁶. Only two point-mutations in the conserved residues (Y3.33C, A5.46G) were shown to be required to creative a CNO-selective extracellular domain in hM4D11. By removing the sequence elements of hM4D that interact with G-proteins and fusing this sequence to the candidate GPCRs (hM4DECD-GPCR), it is expected that a chimeric GPCR will be generated that will be activated by CNO but coupled to G-protein cascades of the candidate GPCR. With these hM4DECD-GPCR fusions, which subtype-restricted GPCRs are coupled to G_{ai/o} will be determined by using established assays to measure reduction in cAMP levels in heterologous cells when stimulated with CNO. cAMP levels will be measured using commercially available, and previously published¹², heterologous HEK293T cells transfections systems (pGloSensor™-22F, Promega). In this assay, a cAMP binding domain is fused to a circularly permuted for of firefly luciferase. When cAMP levels increase, the N- and C-termini of the circularly permuted firefly luciferase are joined, leading to an increase in luminescence. The degree of luminescence is directly proportional to cAMP levels. This assay will provide a reliable, quantitative, and rapid assay for determining whether the candidate GPCRs are Gallo-coupled.

1d. Test the Suppressive Influence of Identified G_{ai/o}-Coupled GPCRs on DRG Neuron Excitability

“Proof-of-principle” experiments will be done to ask if activation of the most promising Gallo coupled subtype-restricted GPCRs can indeed silence sensory neurons. For this, the G_{ai/o}-coupled hM4DECD-GPCR chimera fusion proteins, described in Experiment 1c above, will be used and their activation results in reduction of nociceptor or pruriceptor electrical excitability in vitro will be determined. This will be accomplished by transducing mouse DRG neurons with the chimeric receptors using established AAV transduction protocols in the lab, and treat the neurons with CNO to selectively activate the receptors and measure excitability using standard calcium imaging and whole cell electrophysiological recordings of CGRP+ DRG neurons, which are also routine for the laboratory. It is anticipated that those G_{ai/o}-coupled GPCRs that reduce nociceptor excitability in a CNO-dependent manner will represent a highly curated list of new candidate targets for ligands/agonist identification for treating pain and/or chronic itch. Promising candidates will be chosen from this curated list for subsequent drug screens, using the criteria outlined in the rationale section of this Example.

Milestones and Anticipated Results for Experiment 1.

Experiment 1a. The GPCR Candidate Master List will be completed and in situ hybridization experiments will be done to confirm sensory neuron subtype expression. It is anticipated that this will reveal more than 60 GPCRs that are candidate targets of new pain and itch therapies. Experiment 1b. Testing whether candidate GPCRs are expressed in human DRGs. There is a high degree of conservation of human and rodent DRG gene expression, and so it is anticipated that ˜40-50 GPCRs will remain. Experiment 1c. Determining whether candidate GPCRs are coupled to G_{ai/o}. Since approximately 40% of GPCRs are estimated to be coupled to G_{ai/o} signaling pathways, it is anticipated that ˜15-20 of the ˜40-50 GPCRs expressed in the DRG will be “subtype-select” G_{ai/o}-coupled GPCRs, which will be selected for further functional analysis. Experiment 1d. Experiments that test the suppressive influence of top candidate G_{ai/o}-coupled GPCRs on DRG neuron excitability will be done to test efficacy of GPCRs. A GPCR that suppresses neuronal firing will become a candidate, whereas one that does not suppress neuronal firing would be eliminated from further consideration.

### Experiment 2

Identify small molecule ligands for nociceptor subtype restricted G_{ai/o}-coupled GPCRs.

Rationale

The current standard of care for most pain disorders consists of non-steroidal anti-inflammatory (NSAIDs), serotonin and norepinephrine reuptake inhibitors (SNRIs), anticonvulsants, or opioid analgesics. Many of these treatments carry significant side effects or have high rates of abuse/addictive potential which dramatically diminish their usefulness. Furthermore, many of the current treatments act in the brain and central nervous system. As a result, there is a significant unmet need for therapeutics that are efficacious, safe, and non-addictive alternatives for pain management. This approach, which involves identifying new agonists for nociceptor and pruriceptor subtype specific G_{ai/o}-coupled GPCRs, serves as a direct solution to these issues for several reasons:

1. the primary site of action for these agonists will be peripheral sensory neurons, which have not been a major pharmacological target of pain therapies;

2. the agonists will target select GPCRs and thus all or specific subsets of nociceptors or pruriceptors, thereby leaving other sensory signals that underlie touch and proprioception unaltered, and;

3. future med-chem approaches can be used to generate agonists that are peripherally restricted so that they are bioavailable to peripheral neurons but do not enter the central nervous system, diminishing any potential brain-related side effects.

Here, the workflow that aims to identify new agonists that specifically activate the most compelling sensory neuron subtype specific G_{ai/o}-coupled GPCRs identified in Experiment 1 is outlined.

2a. Testing Candidate Agonists

First, the ability of known agonists for G_{ai/o}-coupled GPCRs that have already been found to be expressed in nociceptor and/or pruriceptor subtypes for reducing behavioral responses to painful and itch stimuli will be tested, including measures of mechanical allodynia in models of neuropathic pain¹³. For example, whether serotonin analogs are effective at suppressing behavioral and physiological responses to itch compounds by silencing pruriceptors, which selectively express Htr1f, a G_{ai/o}-coupled GPCR expressed exclusively in pruriceptors (FIG. 28) will be tested. Additionally, whether the selective Htr1f agonists 5-n-Butyryloxy-DMT14 and LY-33437015 suppresses itch responses, using routine measures or animal behavior following skin injections of structurally-distinct itch compounds, including chloroquine and histamine will be tested.

2b. Using a Combination of In Vitro and In Vivo Based Assays, Candidate Agonists for Promising G_{ai/o} GPCRs Will be Identified.

High-throughput screens for GPCR agonists will be performed using in vitro heterologous cell cultures systems and follow up positive hits with efficacy and specificity using in vivo assays. A five-stage approach is proposed to identifying promising candidate agonists, identified as Screening Stages 1-5, described below.

Screening Stage 1: Identifying Candidate Agonists for the High-Value GPCRs Identified.

In this stage, commercially available cell lines (Euofins) that stably express the GPCR of interest will be used. Of particular value, these GPCRs expressed in stable cell lines are fused in frame with a small enzyme donor fragment ProLink™ (PK) and co-expressed in cells stably expressing a fusion protein of β-arrestin and the larger, N-terminal deletion mutant of β-galactosidase (called enzyme acceptor or EA). Activation of the GPCR stimulates binding of β-arrestin to the PK-tagged GPCR and forces complementation of the two enzyme fragments, resulting in the formation of an active β-galactosidase enzyme, which can be easily quantified using chemiluminescent approaches. The reagents for performing this assay are standardized and commercially available through Eurofins. In collaboration with the ICCB-Longwood Screening Facility, libraries containing over 500,000 small molecules will be screened, with more compounds continuously becoming available (see letter for support from Drs. Caroline Shamu and Jennifer Smith of the ICCB-Longwood Screening Facility). Notably, the β-arrestin based β-galactosidase assay is readily compatible with 96- or 384-well formats for simple, high-throughput compound screening.

Screening Stage 2: Selectivity Determination with Broad Panel Profiling.

Next, hits that activate the GPCR of interest in Stage 1 will be taken, and the selectivity of the identified small molecule in comparison to a broad panel of other GPCRs will be determined. Here, a curated panel of 168 GPCRs made by Eurofins that cover 60 different receptor families (gperMAX™ GPCR Assay Panel) will be used. Through Eurofins, each compound will be tested at three different concentrations for their ability to activate 168 distinct GPCRs. Candidates that show maximal and specific activation of only the GPCR of interest, and not other GPCRs, will pass this stage.

Screening Stage 3: Candidate Agonists Will be Tested to Determine if they can Reduce Pain/Itch Related Behaviors in Mice.

The efficacy of agonists identified in reducing pain behaviors in established animal models will be tested. The ability of identified agonists to block painful mechanical hypersensitivity (allodynia) using the spared nerve injury (SNI) model^{13,16} will also be tested. Additionally, PDN will be induced using the established protocol of depleting pancreatic β cells using chemically-induced streptozotocin (STZ) based models¹⁷ and the ability of identified agonists to reduce thermal and mechanical hyperalgesia in this model of PDN will be tested. As the most robust models of itch in mice are acute, the ability of the newly identified agonists to block the robust paradigms of acutely induced chloroquine or histamine induced itch will be tested. Compounds will be intraperitoneally injected using a range of doses and time points, and all behavioral assays will be done using 6 or more mice per treatment group.

Screening Stage 4: Test Specificity by Determining Whether Candidate Agonist Effects are Lost in Receptor Knockout Mice.

The great power of the mouse as a model system is that it affords the ability to do genetic experiments that address gene function in vivo. The specificity of newly identified agonists towards their GPCR targets will be tested by asking whether any inhibitory effects of the new GPCR agonists towards pain or itch behaviors are lost in mice in which the gene encoding the target GPCR is selectively deleted in the DRG. Mice lacking the GPCR of interest will be generated using standard gene-targeting strategies routinely performed in the lab. These GPCR conditional knockout experiments and behavioral measures will establish whether the newly identified compounds can effectively reduce pain or itch behaviors using the aforementioned in vivo mouse models by acting on their GPCR targets expressed in sensory neurons.

Screening Stage 5: Optimization of Candidate Agonist to Improve Bioavailability or Affinity.

Candidate molecules that pass Stages 1-4 will be considered for further development through partnering with medicinal chemists or biotech or pharmaceutical companies with expertise in med-chem for compound optimization, and for measurements of compound PK, oral availability, and toxicity.

Overview and Future Directions.

In the future, and depending on timing, the consequences of novel compound treatments will be tested using a range of behavioral assays routinely done in the laboratory. These will include gait analysis, touch sensitivity, temperature preference, measures of anxiety, general motor function, mating behaviors, weight, GI function and colonic pain, and more. Furthermore, using in vivo functional Ca2+ imaging (GCaMP7s), also routinely done in the lab, the effects of drug treatment on neuronal activity in nociceptor and pruriceptor populations will be measured. Together, the proposed and future studies will (a) define new candidate target G_{ai/o}-coupled GPCRs expressed in nociceptors or pruriceptors, (b) identify GPCR agonists that silence nociceptor and pruriceptor subtypes, and (c) allow for the movement towards developing these new drugs to treat different types of pain and chronic itch.

Anticipated Results for Experiment 2

Candidate GPCR agonists will be tested for efficacy in pain and itch behavioral assays. A few candidate agonists are available to test immediately. Efficacy in the absence of unwanted side effects will determine whether a candidate is a go/no go for further development. Experiment 2b. First, high-throughput assays for G_{ai/o}-coupled GPCRs will be designed, making use of cell lines purchased from the Eurofins collection of ProLink GPCRs. The ProLink β-galactosidase complementation assay will be used to perform high-throughput screens to identify agonists of the top two or three most promising candidate G_{ai/o} GPCRs. As an example, if GPR149 (FIG. 28) is found in Experiment 1 to be coupled to G_{ai/o}, expressed in human DRGs, and has low or undetectable levels of expression in other peripheral tissues, then this GPCR would be an ideal candidate to put forth for drug screens. The GPR149 cell line for the high-throughput β-galactosidase complementation assay is available from Eurofins. If compounds that activate GPR149 are identified, then these would be used in behavioral experiments that test efficacy. In this case, IP would be established and compounds would be considered for new chemistry and further testing, in consultation with our BBA colleagues. Hits from screens will be tested using standard behavioral measures of pain and itch. Compounds that attenuate responses to painful and itch stimuli will be further considered, for new chemistry and further testing, in consultation with BBA colleagues.

### REFERENCES FOR EXAMPLE 2

1. Julius, D. TRP channels and pain. Annu Rev Cell Dev Biol 29, 355-384, doi:10.1146/annurev-cellbio-101011-155833 (2013).
2. Basbaum, A. I., Bautista, D. M., Scherrer, G. & Julius, D. Cellular and molecular mechanisms of pain. Cell 139, 267-284, doi:10.1016/j.cell.2009.09.028 (2009).
3. Sharma, N. et al. The emergence of transcriptional identity in somatosensory neurons. Nature 577, 392-398, doi:10.1038/s41586-019-1900-1 (2020).
4. Luscher, C. & Slesinger, P. A. Emerging roles for G protein-gated inwardly rectifying potassium (GIRK) channels in health and disease. Nat Rev Neurosci 11, 301-315, doi:10.1038/nrn2834 (2010).
5. Vardy, E. et al. A New DREADD Facilitates the Multiplexed Chemogenetic Interrogation of Behavior. Neuron 86, 936-946, doi:10.1016/j.neuron.2015.03.065 (2015).
6. Armbruster, B. N., Li, X., Pausch, M. H., Herlitze, S. & Roth, B. L. Evolving the lock to fit the key to create a family of G protein-coupled receptors potently activated by an inert ligand. Proc Natl Acad Sci USA 104, 5163-5168, doi:10.1073/pnas.0700293104 (2007).
7. Yudin, Y. & Rohacs, T. Inhibitory Gi/O-coupled receptors in somatosensory neurons: Potential therapeutic targets for novel analgesics. Mol Pain 14, 1744806918763646, doi:10.1177/1744806918763646 (2018).
8. Ray, P. et al. Comparative transcriptome profiling of the human and mouse dorsal root ganglia: an RNA-seq-based resource for pain and sensory neuroscience research. Pain 159, 1325-1345, doi:10.1097/j.pain.0000000000001217 (2018).
9. Zeisel, A. et al. Molecular Architecture of the Mouse Nervous System. Cell 174, 999-1014 e1022, doi:10.1016/j.cell.2018.06.021 (2018).
10. Cao, J. et al. The single-cell transcriptional landscape of mammalian organogenesis. Nature 566, 496-502, doi:10.1038/s41586-019-0969-x (2019).
11. Urban, D. J. & Roth, B. L. DREADDs (designer receptors exclusively activated by designer drugs): chemogenetic tools with therapeutic utility. Annu Rev Pharmacol Toxicol 55, 399-417, doi:10.1146/annurev-pharmtox-010814-124803 (2015).
12. Manglik, A. et al. Structure-based discovery of opioid analgesics with reduced side effects. Nature 537, 185-190, doi:10.1038/nature19112 (2016).
13. Decosterd, I. & Woolf, C. J. Spared nerve injury: an animal model of persistent peripheral neuropathic pain. Pain 87, 149-158, doi:10.1016/s0304-3959(00)00276-1 (2000).
14. Klein, M. T., Dukat, M., Glennon, R. A. & Teitler, M. Toward selective drug development for the human 5-hydroxytryptamine 1E receptor: a comparison of 5-hydroxytryptamine 1E and 1F receptor structure-affinity relationships. J Pharmacol Exp Ther 337, 860-867, doi:10.1124/jpet.111.179606 (2011).
15. Wainscott, D. B. et al. [3H]LY334370, a novel radioligand for the 5-HT1F receptor. I. In vitro characterization of binding properties. Naunyn Schmiedebergs Arch Pharmacol 371, 169-177, doi:10.1007/s00210-005-1035-9 (2005).
16. Shields, S. D., Eckert, W. A., 3rd & Basbaum, A. I. Spared nerve injury model of neuropathic pain in the mouse: a behavioral and anatomic analysis. J Pain 4, 465-470, doi:10.1067/s1526-5900(03)00781-8 (2003).
17. Leiter, E. H. Multiple low-dose streptozotocin-induced hyperglycemia and insulitis in C57BL mice: influence of inbred background, sex, and thymus. Proc Natl Acad Sci USA 79, 630-634, doi:10.1073/pnas.79.2.630 (1982).

### Example 3

This example references FIGS. 27-29, as well as the following lists of identified genes with one nodal and two nodal involvement.

Descriptive Summary from Human Gene Database of the Identified Genes with One Nodal Involvement

Somatostatin node-Npy2r (Neuropeptide Y (NPY) receptors) are a family of Gi/o-protein-coupled receptors that are currently divided into four subtypes: Y1, Y2, Y4 and Y5. NPY receptors mediate a diverse range of biological actions including stimulation of food intake and modulation of circadian rhythm.

CGRP-θ node-Mrgpra3 (Mas-related G-protein coupled receptor member A3): Orphan receptor. May be a receptor for RFamide-family neuropeptides such as NPFF and NPAF, which are analgesic in vivo. May regulate nociceptor function and/or development, including the sensation or modulation of pain (By similarity). Activated by the antimalarial drug chloroquine. Mediates chloroquine-induced itch, in a histamine-independent manner.

Mrgprg+ve node-Mrgprd (MAS Related GPR Family Member D): May regulate nociceptor function and/or development, including the sensation or modulation of pain. Functions as a specific membrane receptor for beta-alanine. Beta-alanine at micromolar doses specifically evoked Ca(2+) influx in cells expressing the receptor. Beta-alanine decreases forskolin-stimulated cAMP production in cells expressing the receptor, suggesting that the receptor couples with G-protein G(q) and G(i).

CGRP-η node-Gpr174 (G Protein-Coupled Receptor 174): This family member is classified as an orphan receptor because the cognate ligand has not been identified. This gene encodes a protein belonging to the G protein-coupled receptor superfamily.

CGRP-ε node-Grm3 &Cold thermo node-Grm 5 (Glutamate Metabotropic Receptor 3/5): The metabotropic glutamate receptors are a family of G protein-coupled receptors, that have been divided into 3 groups on the basis of sequence homology, putative signal transduction mechanisms, and pharmacologic properties. Group I includes GRM1 and GRM5 and these receptors have been shown to activate phospholipase C. Group II includes GRM2 and GRM3 while Group III includes GRM4, GRM6, GRM7 and GRM8. Group II and III receptors are linked to the inhibition of the cyclic AMP cascade but differ in their agonist selectivities. Potentially Gi coupled.

CGRP-α node-Htr5a (5-Hydroxytryptamine Receptor 5): The neurotransmitter serotonin (5-hydroxytryptamine, 5-HT) has been implicated in a wide range of psychiatric conditions and also has vasoconstrictive and vasodilatory effects. The gene described in this record is a member of 5-hydroxytryptamine (serotonin) receptor family and encodes a multi-pass membrane protein that functions as a receptor for 5-hydroxytryptamine and couples to G-proteins. This protein has been shown to function in part through the regulation of intracellular Ca2+ mobilization. Potentially Gs coupled.

Descriptive Summary from Human Gene Database of the Identified Genes with Two Nodal Involvement

CGRP-ε & CGRP-α node-Npy1r (Neuropeptide Y Receptor Y1): This gene belongs to the G-protein-coupled receptor superfamily. The encoded transmembrane protein mediates the function of neuropeptide Y (NPY), a neurotransmitter, and peptide YY (PYY), a gastrointestinal hormone. The encoded receptor undergoes fast agonist-induced internalization through clathrin-coated pits and is subsequently recycled back to the cell membrane. Activation of Y1 receptors may result in mobilization of intracellular calcium and inhibition of adenylate cyclase activity. Potentially Gi coupled.

CGRP-α & Cold thermo-Ntsr2 (Neurotensin Receptor 2): belongs to the G protein-coupled receptor family that activate a phosphatidylinositol-calcium second messenger system. Binding and pharmacological studies demonstrate that this receptor binds neurotensin as well as several other ligands already described for neurotensin NT1 receptor. However, unlike NT1 receptor, this gene recognizes, with high affinity, levocabastine, a histamine H1 receptor antagonist previously shown to compete with neurotensin for low-affinity binding sites in brain. These activities suggest that this receptor may be of physiological importance and that a natural agonist for the receptor may exist.

Somatostatin & CGRP-ε node-Htr1a (5-Hydroxytryptamine Receptor 1A): This gene encodes a G protein-coupled receptor for 5-hydroxytryptamine (serotonin), and belongs to the 5-hydroxytryptamine receptor subfamily. Inactivation of this gene in mice results in behavior consistent with an increased anxiety and stress response. Mutation in the promoter of this gene has been associated with menstrual cycle-dependent periodic fevers. Also functions as a receptor for various drugs and psychoactive substances. Ligand binding causes a conformation change that triggers signaling via guanine nucleotide-binding proteins (G proteins) and modulates the activity of down-stream effectors, such as adenylate cyclase. Beta-arrestin family members inhibit signaling via G proteins and mediate activation of alternative signaling pathways. Signaling inhibits adenylate cyclase activity and activates a phosphatidylinositol-calcium second messenger system that regulates the release of Ca(2±) ions from intracellular stores. Plays a role in the regulation of 5-hydroxytryptamine release and in the regulation of dopamine and 5-hydroxytryptamine metabolism. Potentially Gi coupled

### EQUIVALENTS AND SCOPE

In the articles such as “a,” “an,” and “the” may mean one or more than one unless indicated to the contrary or otherwise evident from the context. Embodiments or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claims that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the embodiments. Because such embodiments are deemed to be known to one of ordinary skill in the art, they may be excluded even if the exclusion is not set forth explicitly herein. Any particular embodiment of the invention can be excluded from any embodiment, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended embodiments. Those of ordinary skill in the art will appreciate that various changes and modifications to this description may be made without departing from the spirit or scope of the present invention, as defined in the following embodiments.

1. A method of screening to identify an agent that selectively inhibits primary nociceptors to attenuate pain perception, said method comprising contacting a G-Protein Coupled Receptor (GPCR) that is selectively expressed in said nociceptors relative to other subtypes of somatosensory neurons with a candidate agent and detecting whether said candidate agent activates the G-Protein Coupled Receptor.

2. The method of claim 1, wherein the G-Protein Coupled Receptor is highly expressed in said nociceptors but expressed at low levels in other subtypes of somatosensory neurons.

3. The method of claim 2, wherein the G-Protein Coupled Receptor is expressed at low levels in peripheral tissues and/or brain.

4. The method of claim 1, wherein the G-Protein Coupled Receptor is coupled to the G_{ai/o}-signaling pathway.

5. The method of claim 1, wherein the G-Protein Coupled Receptor exhibits a conserved pattern of expression between rodent and human dorsal root ganglia (DRG).

6. The method of claim 1, wherein the G-Protein Coupled Receptor is selected from the group consisting of ADGRA1, ADGRD1, ADGRE5, ADGRF5, ADORA2A, ADORA2B, ADRA2A, ADRA2C, AGTR1A, AGTR1B, AGTRAP, AVPR1A, CALCRL, CELSR2, CESLR3, CHRM1, CRCP, CYSLTR2, DRD1, F2RL1, F2RL2, FZD3, FZD5, FZD8, GALR1, GHSR, GPR35, GPR149, GPR156, GPR173, GPR174, GPR19, GPR4, GRM5, GRM7, HCRTR1, HTR1B, HTR1F, HTR4, HTR5A, LPAR1, LPAR3, MRGPRA1, MRGPRA3, MRGPRA4, MRGPRB4, MRGPRB5, MRGRPD, MRGPRE, MRGPRX1, NPY1R, NYP2R, OGFR, OLFR139, OPN3, OPRK1, OPRM1, OXTR, PROKR2, PTAFR, PTGDR, PTGER1, PTGER2, PTGER3, PTGFR, PTGIR, RAMP3, RHO, S1PR1, S1PR2, S1PR3, SSTR2, VMN1R85, and VMN1R89.

7. The method of claim 1, wherein the G-Protein Coupled Receptor is ADRA2C.

8. The method of claim 1, wherein the G-Protein Coupled Receptor is GPR35.

9. The method of claim 1, wherein the G-Protein Coupled Receptor is GPR149.

10. The method of claim 1, wherein the G-Protein Coupled Receptor is HTR1B.

11. The method of claim 1, wherein the G-Protein Coupled Receptor is PTGFR.

12. The method of claim 1, wherein the candidate agent is a small molecule compound, peptide, antigen binding protein, or nucleic acid molecule.

13. The method of claim 1, wherein the method of screening comprises an in vitro based assay.

14. The method of claim 13, wherein the in vitro based assay comprises contacting the candidate agent with a cell line expressing said G-Protein Coupled Receptor and detecting activation of the G-Protein Coupled Receptor.

15. The method of claim 1, wherein the method of screening comprises an in vivo based assay.

16. The method of claim 1, further comprising confirming the selectivity of the candidate agent by detecting no or minimal activation of one or more known control G-Protein Coupled Receptors by the candidate agent.

17. The method of claim 15, wherein the in vivo based assay comprises testing the efficacy of said candidate agent in attenuating pain perception in an animal model.

18. The method of claim 1, wherein the agent is a small molecule compound.

19. The method of claim 1, wherein the agent is a peptide.

20. The method of claim 1, wherein the agent is an antigen binding protein.

21. The method of claim 4, wherein the agent inhibits the G_{ai/o}-signaling pathway.

22. A method of screening to identify an agent that selectively inhibits primary pruriceptors to attenuate itch perception, said method comprising contacting a G-Protein Coupled Receptor that is selectively expressed in said pruriceptors relative to other subtypes of somatosensory neurons with a candidate agent and detecting whether said candidate agent activates the G-Protein Coupled Receptor.

23. The method of claim 22, wherein the G-Protein Coupled Receptor is highly expressed in said pruriceptors but expressed at low levels in other subtypes of somatosensory neurons.

24. The method of claim 22, wherein the G-Protein Coupled Receptor is expressed at low levels in peripheral tissues and/or brain.

25. The method of claim 22, wherein the G-Protein Coupled Receptor is coupled to the G_{ai/o}-signaling pathway.

26. The method of claim 22, wherein the G-Protein Coupled Receptor exhibits a conserved pattern of expression between rodent and human dorsal root ganglia (DRG).

27. The method of claim 22, wherein the G-Protein Coupled Receptor is selected from the group consisting of ADGRA1, ADGRD1, ADGRE5, ADGRF5, ADORA2A, ADORA2B, ADRA2A, ADRA2C, AGTR1A, AGTR1B, AGTRAP, AVPR1A, CALCRL, CELSR2, CESLR3, CHRM1, CRCP, CYSLTR2, DRD1, F2RL1, F2RL2, FZD3, FZD5, FZD8, GALR1, GHSR, GPR35, GPR149, GPR156, GPR173, GPR174, GPR19, GPR4, GRM5, GRM7, HCRTR1, HTR1B, HTR1F, HTR4, HTR5A, LPAR1, LPAR3, MRGPRA1, MRGPRA3, MRGPRA4, MRGPRB4, MRGPRB5, MRGRPD, MRGPRE, MRGPRX1, NPY1R, NYP2R, OGFR, OLFR139, OPN3, OPRK1, OPRM1, OXTR, PROKR2, PTAFR, PTGDR, PTGER1, PTGER2, PTGER3, PTGFR, PTGIR, RAMP3, RHO, S1PR1, S1PR2, S1PR3, SSTR2, VMN1R85, and VMN1R89.

28. The method of claim 22, wherein the G-Protein Coupled Receptor is ADRA2C.

29. The method of claim 22, wherein the G-Protein Coupled Receptor is GPR35.

30. The method of claim 22, wherein the G-Protein Coupled Receptor is GPR149.

31. The method of claim 22, wherein the G-Protein Coupled Receptor is HTR1B.

32. The method of claim 22, wherein the G-Protein Coupled Receptor is PTGFR.

33. The method of claim 22, wherein the candidate agent is a small molecule compound.

34. The method of claim 22, wherein the method of screening comprises an in vitro based assay.

35. The method of claim 34, wherein the in vitro based assay comprises contacting the candidate agent with a cell line expressing said G-Protein Coupled Receptor and detecting activation of the G-Protein Coupled Receptor.

36. The method of claim 22, wherein the method of screening comprises an in vivo based assay.

37. The method of claim 22, further comprising confirming the selectivity of the candidate agent by detecting no or minimal activation of one or more known control G-Protein Coupled Receptors which are expressed in cells other than pruriceptors.

38. The method of claim 36, wherein the in vivo based assay comprises testing the efficacy of said candidate agent in attenuating itch perception in an animal model.

39. The method of claim 22, wherein the candidate agent is a small molecule compound.

40. The method of claim 22, wherein the method of screening comprises an in vitro based assay.

41. The method of claim 40, wherein the in vitro based assay comprises contacting the candidate agent with a cell line expressing said G-Protein Coupled Receptor and detecting activation of the G-Protein Coupled Receptor.

42. The method of claim 22, wherein the method of screening comprises an in vivo based assay.

43. The method of claim 22, further comprising confirming the selectivity of the candidate agent by detecting no or minimal activation of one or more known control G-Protein Coupled Receptors by the candidate agent.

44. The method of claim 42, wherein the in vivo based assay comprises testing the efficacy of said candidate agent in attenuating pain perception in an animal model.

45. The method of claim 22, wherein the agent is a nucleic acid molecule.

46. The method of claim 22, wherein the agent is a peptide.

47. The method of claim 22, wherein the agent is an antigen binding protein.

48. The method of claim 25, wherein the agent inhibits the G_{ai/o}-signaling pathway.
